(19) United States
(12) Patent Application Publication
(10) Pub. No.: US 2010/0016365 A1
(43) Pub. Date: Jan. 21, 2010

(54) SUBSTITUTED 4-AMINO-PIPERIDINES

(75) Inventors: Thomas G. Gant, Carlsbad, CA (US); Sepehr Sarshar, Cardiff by the Sea, CA (US)

Correspondence Address: GLOBAL PATENT GROUP - APX, 10411 Clayton Road, Suite 304, ST. LOUIS, MO, 63131, US

(73) Assignee: AUSPEX PHARMACEUTICALS, INC., Vista, CA (US)

(21) Appl. No.: 12/504,792

(22) Filed: Jul. 17, 2009

Related U.S. Application Data
(60) Provisional application No. 61/081,572, filed on Jul. 17, 2008.

(51) Int. Cl. A61K 31/454; C07D 401/06; A61K 31/4468; A61P 29/00; A61P 19/02; A61P 9/10; C07D 211/56; A61K 31/4535

(52) U.S. Cl. 514/326; 546/210; 514/329; 546/224

(57) ABSTRACT

The present invention relates to new substituted 4-amino-piperidine opioid receptor modulators, pharmaceutical compositions thereof, and methods of use thereof.

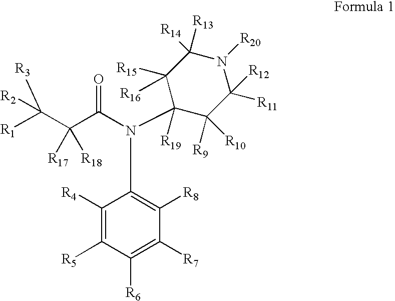

This application claims the benefit of priority of U.S. provisional application No. 61/081,572, filed Jul. 17, 2008, the disclosure of which is hereby incorporated by reference as if written herein in its entirety.

Disclosed herein are new substituted 4-amino-piperidine compounds, pharmaceutical compositions made thereof, and methods to modulate opioid receptor activity with such pharmaceuticals for the treatment of disorders in a subject, such as preoperative pain, intraoperative pain, postoperative pain, partuition, oncological pain, neuropathy, osteoarthritis, rheumatoid arthritis, fibromyalgia, pancreatitis, herniated discs, migraine, trigeminal neuralgia, sciatica, myocardial infarctions, any procedure which requires anesthesia or sedation, any disorder associated with moderate to severe acute or chronic pain, and/or any disorder ameliorated by opioid receptor modulation.

Fentanyl (Duragesic®, Fentora®, Actiq®, Ionsys™) N-(1-(2-phenylethyl)-4-piperidinyl)-N-phenyl-propanamide, sufentanil (Suffenta®) N-[4-(methoxymethyl)-1-(2-thiophen-2-ylethyl)-4-piperidyl]-N-phenyl-propanamide, and alfentanil (Alfenta®) N-[1-[2-(4-ethyl-5-oxo-1,4-dihydrotetrazol-1-yl)ethyl]-4-(methoxymethyl)-4-piperidyl]-N-phenyl-propanamide, are putative opioid receptor agonists. They are used for treating acute and chronic pain. Fentanyl, a highly lipid-soluble synthetic opioid related to the phenylpiperidines, is estimated to be 80 times more potent than morphine as an analgesic (Paix et al., Pain 1995, 63(2), 263-9). Fentanyl is usually administered intravenously as a supplement to general anesthesia (McClain et al., Clin Pharmacol Ther 1980, 28(1), 106-14; Sano et al., Vet Anaesth Analg 2006, 33(4), 266-73). However, due to fentanyl's low molecular weight and high lipophilicity, it can be effectively administered transdermally and through the mucosal membranes of the mouth, nose and respiratory tract (Grond et al., Clin Pharmacokinet 2000, 38(1), 59-89). Sufentanil, a synthetic opioid analgesic drug, is approximately 5 to 10 times more potent than fentanyl (Rolly et al., Acta Anaeshesiol Belg 1991, 30(4), 247-54). Sufentanil is generally used in operating suites and critical care wards, where a fast acting analgesic is required. Sufentanil can be administered intravenously or epidurally (Frikha et al., Middle East J Anesthesiol 2007, 19(1), 87-96). A Sufentanil based transdermal patch is currently under development (Fischer et al., Anesthesiology 2003, 99(4), 929-37). Alfentanil is a very short acting opioid. Alfentanil exerts an analagesic effect anywhere from 5 to 10 minutes after administration of a single intravenous dose. Alfentanil provides faster acting analgesia than fentanyl or sufentanil (Scott et al., Anesthesiology 1985, 62(3), 234-41). While alfentanil gives less cardiovascular complications than fentanyl or sufentanil, it tends to give stronger respiratory depression. In contrast to fentanyl, the duration of effect of a single dose of alfentanil depends more on total body clearance than on redistribution to tissues, probably because of alfentanil's lower lipid solubility (Tegeder et al., Clin Pharmacokinet 1999, 37(1), 17-40). Fentanyl, sulfentanil and/or alfentanil are commonly prescribed for disorders such as: pre-operative pain (Dsida et al., Anesth Analg. 1998, 86(1), 66-70), intra-operative pain (Thomson et al., J Cardiothorac Vasc Anesth. 2000, 14(6), 652-6), post-operative pain (Grond et al., Br J Anaesth 2007, 98(6), 806-815), partuition (Frikha et al., Middle East J Anesthesiol 2007, 19(1), 87-96), oncological pain (Ripamonti et al., Support Care Cancer 2006, 14(5), 400-7), neuropathy (Simpson et al., Drugs Aging 2006, 23(5), 357-75), osteoarthritis (Langford et al., Arthritis Rheum 2006, 54, 1829-37), rheumatoid arthritis (Pavelka et al., Curr Med Res Opin 2004, 20(12), 1967-77), fibromyalgia (Callejas Rubio et al., Med Clin (Barc) 2003, 120(9), 358-9; Price et al., Pain 2002, 99(1-2), 49-59), pancreatitis (Stevens et al., Appl Nurs Res. 2002, 15(2), 102-110), herniated discs (Rainov et al., Acta Neurochir (Wien) 1996, 138(1), 33-39; Hassi et al., Cah Anesthesiol 1995, 43(1), 21-5), migraine (Landy S H, Headache 2004, 44(8), 762-6), trigeminal neuralgia (Liu et al., Neurosurgery Clinics of North America 2004, 15(3), 319-334), sciatica (Grilo et al., Joint Bone Spine 2002, 69(5), 491-4), and myocardial infarctions (Myles et al., Anesth Analg 2002, 95(4), 805-812). While other opioids can be used as analgesics, i.e. tramadol, morphine, codeine, and the like, they generally have additional deleterious side effects, such as modulating the reuptake of neurotransmitters and physical and psychological dependency.

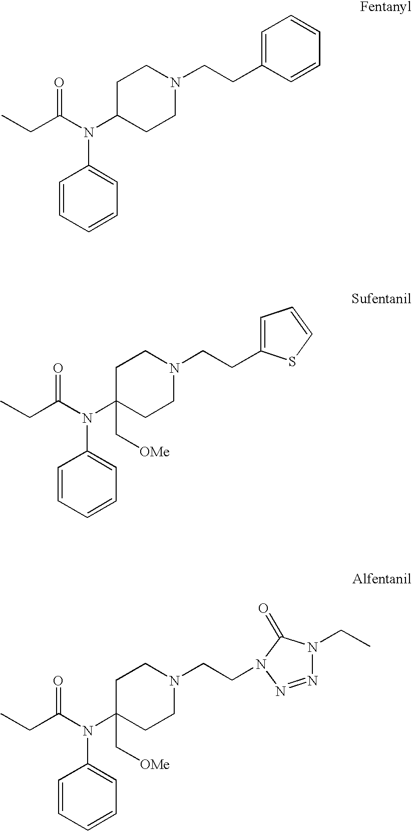

Fentanyl was found to undergo N-dealkylation and hydroxylation to produce metabolites that were both inactive and nontoxic (McClain et al., Clin Pharmacol Ther 1980, 28(1), 106-14). The main metabolites, 4-N-(N-propionylanilino)-piperidine and 4-N-(N-hydroxypropionylanilino)-piperidine, and a small amount of unchanged drug (<10%), are excreted in the urine (Goromaru et al., Anaesthesiology 1984, 61(1), 73-7). Fentanyl is eliminated with a half-live of only about 3.5 hours, sulfentanil with a half-live of only about 3 hours, and alfentanil with a half-live of only about 1.5 hours, but there is considerable interpatient variability (Bentley et al., Anesth Analg 1982, 61(12), 968-971; Mather L E, Clin Pharmacokinet 1983, 8(5), 422-46; McClain et al., Clin Pharmacol Ther 1980, 28(1), 106-114; Bovill et al., Anesthesiology 1997, 61(5), 502-6; Lehman et al., Acta Anaesthesiol Scand 1993, 37(2), 176-80; Meuldermans et al., Eur J Clin Pharmacol 1986, 30(2), 217-219). Longer fentanyl and sufentanil half lives were seen in patients undergoing cardiac or abdominal aortic surgery (Koska et al., Clin Pharmacol Ther 1981, 29(1), 100-5; Hudson et al., Anesthesiology 1986, 8(5), 334-8; Howie et al., J Cardiothorac Vasc Anesth 1991, 5(5), 485-9).

### Deuterium Kinetic Isotope Effect

In order to eliminate foreign substances such as therapeutic agents, the animal body expresses various enzymes, such as the cytochrome P₄₅₀ enzymes (CYPs), esterases, proteases, reductases, dehydrogenases, and monoamine oxidases, to react with and convert these foreign substances to more polar intermediates or metabolites for renal excretion. Such metabolic reactions frequently involve the oxidation of a carbon-hydrogen (C—H) bond to either a carbon-oxygen (C—O) or a carbon-carbon (C—C) π-bond. The resultant metabolites may be stable or unstable under physiological conditions, and can have substantially different pharmacokinetic, pharmacodynamic, and acute and long-term toxicity profiles relative to the parent compounds. For most drugs, such oxidations are generally rapid and ultimately lead to administration of multiple or high daily doses.

The relationship between the activation energy and the rate of reaction may be quantified by the Arrhenius equation, k=Ae^{-Eact/RT}. The Arrhenius equation states that, at a given temperature, the rate of a chemical reaction depends exponentially on the activation energy (E_{act}).

The transition state in a reaction is a short lived state along the reaction pathway during which the original bonds have stretched to their limit. By definition, the activation energy E_{act} for a reaction is the energy required to reach the transition state of that reaction. Once the transition state is reached, the molecules can either revert to the original reactants, or form new bonds giving rise to reaction products. A catalyst facilitates a reaction process by lowering the activation energy leading to a transition state. Enzymes are examples of biological catalysts.

Carbon-hydrogen bond strength is directly proportional to the absolute value of the ground-state vibrational energy of the bond. This vibrational energy depends on the mass of the atoms that form the bond, and increases as the mass of one or both of the atoms making the bond increases. Since deuterium (D) has twice the mass of protium (¹H), a C-D bond is stronger than the corresponding C—¹H bond. If a C—¹H bond is broken during a rate-determining step in a chemical reaction (i.e. the step with the highest transition state energy), then substituting a deuterium for that protium will cause a decrease in the reaction rate. This phenomenon is known as the Deuterium Kinetic Isotope Effect (DKIE). The magnitude of the DKIE can be expressed as the ratio between the rates of a given reaction in which a C—¹H bond is broken, and the same reaction where deuterium is substituted for protium. The DKIE can range from about 1 (no isotope effect) to very large numbers, such as 50 or more. Substitution of tritium for hydrogen results in yet a stronger bond than deuterium and gives numerically larger isotope effects

Deuterium (²H or D) is a stable and non-radioactive isotope of hydrogen which has approximately twice the mass of protium (¹H), the most common isotope of hydrogen. Deuterium oxide (D₂O or “heavy water”) looks and tastes like H₂O, but has different physical properties.

When pure D₂O is given to rodents, it is readily absorbed. The quantity of deuterium required to induce toxicity is extremely high. When about 0-15% of the body water has been replaced by D₂O, animals are healthy but are unable to gain weight as fast as the control (untreated) group. When about 15-20% of the body water has been replaced with D₂O, the animals become excitable. When about 20-25% of the body water has been replaced with D₂O, the animals become so excitable that they go into frequent convulsions when stimulated. Skin lesions, ulcers on the paws and muzzles, and necrosis of the tails appear. The animals also become very aggressive. When about 30% of the body water has been replaced with D₂O, the animals refuse to eat and become comatose. Their body weight drops sharply and their metabolic rates drop far below normal, with death occurring at about 30 to about 35% replacement with D₂O. The effects are reversible unless more than thirty percent of the previous body weight has been lost due to D₂O. Studies have also shown that the use of D₂O can delay the growth of cancer cells and enhance the cytotoxicity of certain antineoplastic agents.

Deuteration of pharmaceuticals to improve pharmacokinetics (PK), pharmacodynamics (PD), and toxicity profiles has been demonstrated previously with some classes of drugs. For example, the DKIE was used to decrease the hepatotoxicity of halothane, presumably by limiting the production of reactive species such as trifluoroacetyl chloride. However, this method may not be applicable to all drug classes. For example, deuterium incorporation can lead to metabolic switching. Metabolic switching occurs when xenogens, sequestered by Phase I enzymes, bind transiently and re-bind in a variety of conformations prior to the chemical reaction (e.g., oxidation). Metabolic switching is enabled by the relatively vast size of binding pockets in many Phase I enzymes and the promiscuous nature of many metabolic reactions. Metabolic switching can lead to different proportions of known metabolites as well as altogether new metabolites. This new metabolic profile may impart more or less toxicity. Such pitfalls are non-obvious and are not predictable a priori for any drug class.

Fentanyl, sufentanil, and alfentanil are substituted 4-amino-piperidine -based opioid receptor modulators. The carbon-hydrogen bonds of fentanyl, sufentanil, and alfentanil contain a naturally occurring distribution of hydrogen isotopes, namely ¹H or protium (about 99.9844%), ²H or deuterium (about 0.0156%), and ³H or tritium (in the range between about 0.5 and 67 tritium atoms per 10¹⁸ protium atoms). Increased levels of deuterium incorporation may produce a detectable Kinetic Isotope Effect (KIE) that could affect the pharmacokinetic, pharmacologic and/or toxicologic profiles of such opioid receptor modulators in comparison with the compound having naturally occurring levels of deuterium.

Based on discoveries made in our laboratory, as well as considering the KIE literature, fentanyl, sufentanil and alfentanil are likely metabolized in humans at the N-methylene group. Other potential sites of metabolism include the phenyl group of fentanyl, sufentanil, and alfentanil, and the O-methyl group of sufentanil and alfentanil. The current approach has the potential to prevent or retard metabolism at these sites. Other sites on the molecule may also undergo transformations leading to metabolites with as-yet-unknown pharmacology/toxicology. Limiting the production of such metabolites has the potential to decrease the danger of the administration of such drugs and may even allow increased dosage and concomitant increased efficacy. All of these transformations, among other potential transformations, can and do occur through polymorphically-expressed enzymes. Such polymorphisms may account for the wide variance seen in interpatient phramacodynamic responses. Further, it is quite typical for disorders ameliorated by the present invention, such as oncological pain, to produce symptoms that are best medicated around the clock for extended periods of time. For all of the foregoing reasons, a medicine with a longer half-life may result in greater efficacy and cost savings. Various deuteration patterns can be used to (a) reduce or eliminate unwanted metabolites, (b) increase the half-life of the parent drug, (c) decrease the number of doses needed to achieve a desired effect, (d) decrease the amount of a dose needed to achieve a desired effect, (e) increase the formation of active metabolites, if any are formed, (f) decrease the production of deleterious metabolites in specific tissues, and/or (g) create a more effective drug and/or a safer drug for polypharmacy, whether the polypharmacy be intentional or not. The deuteration approach has the strong potential to slow the metabolism of fentanyl, sufentanil and alfentanil, and attenuate interpatient variability.

Novel compounds and pharmaceutical compositions, certain of which have been found to modulate opioid receptors have been discovered, together with methods of synthesizing and using the compounds, including methods for the treatment of opioid receptor-mediated disorders in a patient by administering the compounds.

In certain embodiments of the present invention, compounds have structural Formula I:

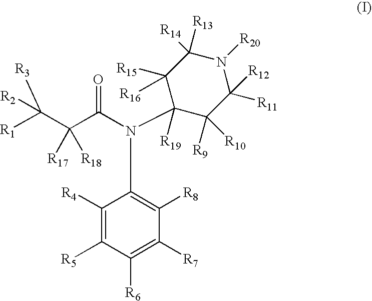

or a pharmaceutically acceptable salt, solvate, or prodrug thereof, wherein:
R₁-R₁₈, R₂₁-R₂₈, R₃₀-R₅₀ are independently selected from the group consisting of hydrogen and deuterium;
R₁₉ is selected from the group consisting of hydrogen, deuterium, and

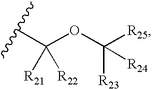

R₂₀ is selected from the group consisting of consisting of hydrogen, deuterium,

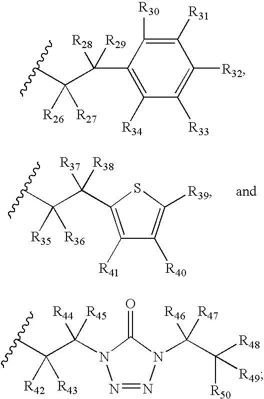

R₂₉ is selected from the group consisting of consisting of hydrogen, deuterium, hydroxyl, and —O-D; and
at least one of R₁-R₅₀ is deuterium.

Certain compounds disclosed herein may possess useful opioid receptor modulating activity, and may be used in the treatment or prophylaxis of a disorder in which opioid receptors play an active role. Thus, certain embodiments also provide pharmaceutical compositions comprising one or more compounds disclosed herein together with a pharmaceutically acceptable carrier, as well as methods of making and using the compounds and compositions. Certain embodiments provide methods for modulating opioid receptors. Other embodiments provide methods for treating an opioid receptor-mediated disorder in a patient in need of such treatment, comprising administering to said patient a therapeutically effective amount of a compound or composition according to the present invention. Also provided is the use of certain compounds disclosed herein for use in the manufacture of a medicament for the treatment of a disorder ameliorated by the modulation of opioid receptors.

The compounds as disclosed herein may also contain less prevalent isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen.

In certain embodiments, the compound disclosed herein may expose a patient to a maximum of about 0.000005% D₂O or about 0.00001% DHO, assuming that all of the C-D bonds in the compound as disclosed herein are metabolized and released as D₂O or DHO. In certain embodiments, the levels of D₂O shown to cause toxicity in animals is much greater than even the maximum limit of exposure caused by administration of the deuterium enriched compound as disclosed herein. Thus, in certain embodiments, the deuterium-enriched compound disclosed herein should not cause any additional toxicity due to the formation of D₂O or DHO upon drug metabolism.

In certain embodiments, the deuterated compounds disclosed herein maintain the beneficial aspects of the corresponding non-isotopically enriched molecules while substantially increasing the maximum tolerated dose, decreasing toxicity, increasing the half-life (T_{1/2}), lowering the maximum plasma concentration (Cₘₐₓ) of the minimum efficacious dose (MED), lowering the efficacious dose and thus decreasing the non-mechanism-related toxicity, and/or lowering the probability of drug-drug interactions.

In another aspect are processes for preparing a compound as disclosed herein or other pharmaceutically acceptable derivative thereof such as a salt, solvate, or prodrug, as an opioid receptor modulator.

In certain embodiments, the compound of Formula I cannot be selected from the group consisting of:

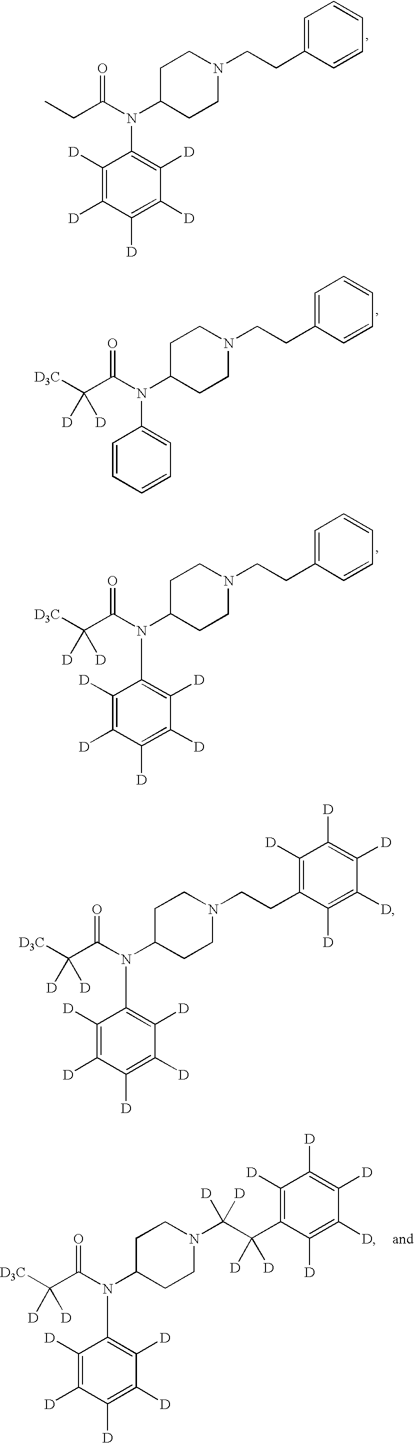

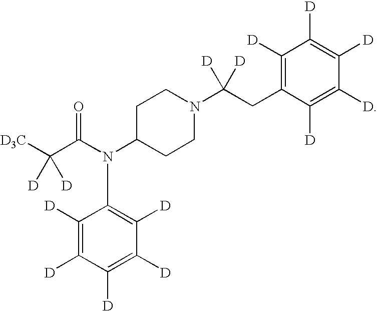

In other embodiments, at least at least one of R₁-R₅₀ independently has deuterium enrichment of no less than about 10%, 50%, 90%, or 98%.

In other embodiments, a pharmaceutical composition comprises a compound disclosed herein together with a pharmaceutically acceptable carrier.

In certain embodiments of the present invention a method of treating a subject suffering from an opioid receptor-mediated disorder comprises the administration of a therapeutically effective amount of a compound as disclosed herein.

In other embodiments said opioid receptor-mediated disorder is selected from the group consisting of preoperative pain, intraoperative pain, postoperative pain, partuition, oncological pain, neuropathy, osteoarthritis, rheumatoid arthritis, fibromyalgia, pancreatitis, herniated discs, migraine, trigeminal neuralgia, sciatica, myocardial infarctions, any procedure which requires anesthesia or sedation, and any disorder associated with moderate to severe acute or chronic pain.

In yet other embodiments, said method further comprises the administration of an additional therapeutic agent.

In further embodiments said therapeutic agent is selected from the group consisting of: opioids, steroidal drugs, local or general anesthetics, sepsis treatments, antibacterial agents, antifungal agents, anticoagulants, thrombolytics, non-steroidal anti-inflammatory agents, antiplatelet agents, norepinephrine reuptake inhibitors (NRIs), dopamine reuptake inhibitors (DARIs), serotonin-norepinephrine reuptake inhibitors (SNRIs), sedatives, norepinephrine-dopamine reuptake inhibitor (NDRIs), serotonin-norepinephrine-dopamine-reuptake-inhibitors (SNDRIs), monoamine oxidase inhibitors, hypothalamic phospholipids, ECE inhibitors, thromboxane receptor antagonists, potassium channel openers, thrombin inhibitors, hypothalamic phospholipids, growth factor inhibitors, P2Y (AC) antagonists, low molecular weight heparins, Factor VIIa Inhibitors and Factor Xa Inhibitors, renin inhibitors, NEP inhibitors, vasopepsidase inhibitors, HMG CoA reductase inhibitors, squalene synthetase inhibitors, fibrates, bile acid sequestrants, anti-atherosclerotic agents, MTP Inhibitors, calcium channel blockers, potassium channel activators, alpha-muscarinic agents, beta-muscarinic agents, antiarrhythmic agents, diuretics, anti-diabetic agents, mineralocorticoid receptor antagonists, growth hormone secretagogues, aP2 inhibitors, phosphodiesterase inhibitors, protein tyrosine kinase inhibitors, antiproliferatives, chemotherapeutic agents, immunosuppressants, anticancer agents and cytotoxic agents, antimetabolites, antibiotics, farnesyl-protein transferase inhibitors, hormonal agents, microtubule-disruptor agents, microtubule-stabilizing agents, plant-derived products, epipodophyllotoxins, taxanes, topoisomerase inhibitors, prenyl-protein transferase inhibitors, cyclosporins, cytotoxic drugs, TNF-alpha inhibitors, anti-TNF antibodies and soluble TNF receptors, cyclooxygenase-2 (COX-2) inhibitors, and miscellaneous agents.

In other embodiments said opioid is selected from the group consisting of morphine, codeine, thebain, diacetylmorphine, oxycodone, hydrocodone, hydromorphone, oxymorphone, nicomorphine, fentanyl, α-methylfentanyl, alfentanil, sufentanil, remifentanyl, carfentanyl, ohmefentanyl, pethidine, ketobemidone, propoxyphene, dextropropoxyphene, methadone, loperamide, pentazocine, buprenorphine, etorphine, butorphanol, nalbufine, levorphanol, naloxone, naltrexone, and tramadol.

In other embodiments said steroidal drug is selected from the group consisting of aldosterone, beclometasone, betamethasone, deoxycorticosterone acetate, fludrocortisone acetate, hydrocortisone (cortisol), prednisolone, prednisone, methylprenisolone, dexamethasone, and triamcinolone.

In other embodiments said local or general anesthetic is selected from the group consisting of diethyl ether, vinyl ether, halothane, chloroform, methoxyflurane, enflurane, trichloroethylene, isoflurane, desflurane, sevoflurane, methohexital, hexobarbital, thiopental, narcobarbital, fentanyl, alfentanil, sufentanil, phenoperidine, anileridine, remifentanil, droperidol, ketamine, propanidid, alfaxalone, etomidate, propofol, hydroxybutyric acid, nitrous oxide, esketamine, metabutethamine, procaine, tetracaine, chloroprocaine, benzocaine, bupivacaine, lidocaine, mepivacaine, prilocaine, butanilicaine, cinchocaine, etidocaine, articaine, ropivacaine, levobupivacaine, cocaine, ethyl chloride, dyclonine, phenol, and capsaicin.

In other embodiments said method further results in at least one effect selected from the group consisting of
a) decreased inter-individual variation in plasma levels of said compound or a metabolite thereof as compared to the non-isotopically enriched compound;
b) increased average plasma levels of said compound per dosage unit thereof as compared to the non-isotopically enriched compound;
c) decreased average plasma levels of at least one metabolite of said compound per dosage unit thereof as compared to the non-isotopically enriched compound;
d) increased average plasma levels of at least one metabolite of said compound per dosage unit thereof as compared to the non-isotopically enriched compound; and
e) an improved clinical effect during the treatment in said subject per dosage unit thereof as compared to the non-isotopically enriched compound.

In other embodiments said method further results in at least two effects selected from the group consisting of:
a) decreased inter-individual variation in plasma levels of said compound or a metabolite thereof as compared to the non-isotopically enriched compound;
b) increased average plasma levels of said compound per dosage unit thereof as compared to the non-isotopically enriched compound;
c) decreased average plasma levels of at least one metabolite of said compound per dosage unit thereof as compared to the non-isotopically enriched compound;
d) increased average plasma levels of at least one metabolite of said compound per dosage unit thereof as compared to the non-isotopically enriched compound; and
e) an improved clinical effect during the treatment in said subject per dosage unit thereof as compared to the non-isotopically enriched compound.

In certain embodiments said method decreases metabolism by at least one polymorphically-expressed cytochrome P450 isoform in said subject per dosage unit thereof as compared to the non-isotopically enriched compound.

In other embodiments said cytochrome P450 isoform is selected from the group consisting of CYP2C8, CYP2C9, CYP2C19, and CYP2D6.

In yet further embodiments said method decreases inhibition of at least one cytochrome P450 or monoamine oxidase isoform in said subject per dosage unit thereof as compared to the non-isotopically enriched compound.

In certain embodiments said cytochrome P450 or monoamine oxidase isoform is selected from the group consisting of CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, CYP51, MAOA, and MAOB.

In certain embodiments, said method reduces a deleterious change in a diagnostic hepatobiliary function endpoint, as compared to the corresponding non-isotopically enriched compound.

In yet other embodiments, said diagnostic hepatobiliary function endpoint is selected from the group consisting of alanine aminotransferase (“ALT”), serum glutamic-pyruvic transaminase (“SGPT”), aspartate aminotransferase (“AST,” “SGOT”), ALT/AST ratios, serum aldolase, alkaline phosphatase (“ALP”), ammonia levels, bilirubin, gamma-glutamyl transpeptidase (“GGTP,” “γ-GTP,” “GGT”), leucine aminopeptidase (“LAP”), liver biopsy, liver ultrasonography, liver nuclear scan, 5′-nucleotidase, and blood protein.

In another embodiment a compound disclosed herein can be used as a medicament.

In a further embodiment a compound disclosed herein can be used in the manufacture of a medicament for the prevention or treatment of a disorder ameliorated by the modulation of opioid receptors.

All publications and references cited herein are expressly incorporated herein by reference in their entirety. However, with respect to any similar or identical terms found in both the incorporated publications or references and those expressly put forth or defined in this document, then those terms definitions or meanings expressly put forth in this document shall control in all respects.

As used herein, the terms below have the meanings indicated.

The singular forms “a,” “an,” and “the” may refer to plural articles unless specifically stated otherwise.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

In representing a range of positions on a structure, the notation “from Rₓ . . . to Rₓₓ” or “Rₓ-Rₓₓ” may be used, wherein x and xx represent numbers. Then unless otherwise specified, this notation is intended to include not only the numbers represented by x and xx themselves, but all the numbered positions that are bounded by x and xx. For example, “from R₁ . . . to R₄” or “R₁-R₄” would, unless otherwise specified, be equivalent to R₁, R₂, R₃, and R₄.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156%, deuterium enrichment at any position in a compound synthesized using non-enriched starting materials is about 0.0156%. The deuterium enrichment can be determined using conventional analytical methods known to one of ordinary skill in the art, including mass spectrometry and nuclear magnetic resonance spectroscopy.

The term “is/are deuterium,” when used to describe a given position in a molecule such as R₁-R₅₀ or the symbol “D,” when used to represent a given position in a drawing of a molecular structure, means that the specified position is enriched with deuterium above the naturally occurring distribution of deuterium. In one embodiment deuterium enrichment is no less than about 1%, in another no less than about 5%, in another no less than about 10%, in another no less than about 20%, in another no less than about 50%, in another no less than about 70%, in another no less than about 80%, in another no less than about 90%, or in another no less than about 98% of deuterium at the specified position.

The term “isotopic enrichment” refers to the percentage of incorporation of a less prevalent isotope of an element at a given position in a molecule in the place of the more prevalent isotope of the element.

The term “non-isotopically enriched” refers to a molecule in which the percentages of the various isotopes are substantially the same as the naturally occurring percentages.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as D-isomers and L-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a linkage between two atoms, or two moieties when the atoms joined by the bond are considered to be part of larger substructure. A bond may be ionic, metallic, or covalent. If covalent, the bond can be either result from the sharing of one pair of electrons, a single bond; a sharing of 2 pairs of electrons, a double bond; a sharing of 3 pairs of electrons, or a triple bond; or sharing of more than 3 pairs of electrons. A dashed line between two atoms in a drawing of a molecule indicates that an additional bond may be present or absent at that position.

The term “disorder” as used herein is intended to be generally synonymous, and is used interchangeably with, the terms “disease,” “syndrome,” and “condition” (as in medical condition), in that all reflect an abnormal condition of the human or animal body or of one of its parts that impairs normal functioning, is typically manifested by distinguishing signs and symptoms.

The terms “treat,” “treating,” and “treatment” are meant to include alleviating or abrogating a disorder or one or more of the symptoms associated with a disorder; or alleviating or eradicating the cause(s) of the disorder itself. As used herein, reference to “treatment” of a disorder is intended to include prevention. The terms “prevent,” “preventing,” and “prevention” refer to a method of delaying or precluding the onset of a disorder; and/or its attendant symptoms, prohibiting a subject from acquiring a disorder or reducing a subject's risk of acquiring a disorder.

The term “therapeutically effective amount” refers to the amount of a compound that, when administered, is sufficient to prevent development of, or alleviate to some extent, one or more of the symptoms of the disorder being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “subject” refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein in reference, for example, to a mammalian subject, such as a human patient.

The term “combination therapy” means the administration of two or more therapeutic agents to treat a therapeutic disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the disorders described herein.

The term “opioid receptor” refers to the class of G-protein coupled receptors that effect GABAergic neurotransmission upon opioid binding. The endogenous opioids are dynorphins, enkephalins, and endorphins. The opioid receptors are ˜40% identical to somatostation receptors (SSTR). There are at least 17 major classes of opioid receptors, although only three are generally spoken of: μ, κ, and δ (mu, kappa and delta). In addition there are two subtypes of μ receptor: μ₁ and μ₂. The pharmacodynamic response to an opioid depends on which receptor it binds, its affinity for that receptor and whether the opioid is an agonist or an antagonist. Activation of the μ-opioid receptors is associated with analgesia, sedation, euphoria, physical dependence and respiratory depression. κ-Opioid receptors are involved with analgesia, but activation also produces marked nausea, dysphoria and psychotomimetic effects. δ-opioid receptor activation produces analgesia. Unless stated otherwise, the term “opioid receptor” includes all subtypes of opioid receptors.

The term “opioid receptor modulator” refers to the ability of a compound disclosed herein to alter the function of an opioid receptor. An opioid receptor modulator may activate the activity of an opioid receptor, may activate or inhibit the activity of an opioid receptor depending on the concentration of the compound exposed to the opioid receptor, or may inhibit the activity of an opioid receptor. Such activation or inhibition may be contingent on the occurrence of a specific event, such as activation of a signal transduction pathway, and/or may be manifest only in particular cell types. The term “opioid receptor modulator” also refers to altering the function of an opioid receptor by increasing or decreasing the probability that a complex forms between an opioid receptor and a natural binding partner. An opioid receptor modulator may increase the probability that such a complex forms between the opioid receptor and the natural binding partner, may increase or decrease the probability that a complex forms between the opioid receptor and the natural binding partner depending on the concentration of the compound exposed to the opioid receptor, and or may decrease the probability that a complex forms between the opioid receptor and the natural binding partner. In some embodiments, modulation of the opioid receptor may be assessed using Receptor Selection and Amplification Technology (R-SAT) as described in U.S. Pat. No. 5,707,798, the disclosure of which is incorporated herein by reference in its entirety.

The terms “opioid receptor-mediated disorder” refers to a disorder that is characterized by abnormal opioid receptor activity or normal opioid receptor receptor activity that, when that activity is modified, leads to the amelioration of other abnormal biological processes. Opioid receptor-mediated disorders may be completely or partially mediated by modulation of opioid receptors. In particular, an opioid receptor-mediated disorder is one in which modulation of an opioid receptor's activity results in some effect on the underlying disorder, e.g., administering an opioid receptor modulator results in some improvement in at least some of the patients being treated.

The term “therapeutically acceptable” refers to those compounds (or salts, prodrugs, tautomers, zwitterionic forms, etc.) which are suitable for use in contact with the tissues of patients without excessive toxicity, irritation, allergic response, immunogenecity, are commensurate with a reasonable benefit/risk ratio, and are effective for their intended use.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenecity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The terms “active ingredient,” “active compound,” and “active substance” refer to a compound, which is administered, alone or in combination with one or more pharmaceutically acceptable excipients or carriers, to a subject for treating, preventing, or ameliorating one or more symptoms of a disorder.

The terms “drug,” “therapeutic agent,” and “chemotherapeutic agent” refer to a compound, or a pharmaceutical composition thereof, which is administered to a subject for treating, preventing, or ameliorating one or more symptoms of a disorder.

The term “release controlling excipient” refers to an excipient whose primary function is to modify the duration or place of release of the active substance from a dosage form as compared with a conventional immediate release dosage form.

The term “nonrelease controlling excipient” refers to an excipient whose primary function do not include modifying the duration or place of release of the active substance from a dosage form as compared with a conventional immediate release dosage form.

The term “prodrug” refers to a compound functional derivative of the compound as disclosed herein and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; Waller et al., Br. J. Clin. Pharmac. 1989, 28, 497-507.

The term “reducing reagent” refers to any reagent that will decrease the oxidation state of an atom in the starting material by either adding a hydrogen to this atom, or adding an electron to this atom, or by removing an oxygen from this atom and as such would be obvious to one of ordinary skill and knowledge in the art. The definition of “reducing reagent” includes but is not limited to: borane-dimethyl sulfide complex, 9-borabicyclo[3.3.1.]nonane (9-BBN), catechol borane, lithium borohydride, lithium borodeuteride, sodium borohydride, sodium borodeuteride, sodium borohydride-methanol complex, potassium borohydride, sodium hydroxyborohydride, lithium triethylborohydride, lithium n-butylborohydride, sodium cyanoborohydride, sodium cyanoborodeuteride, calcium (II) borohydride, lithium aluminum hydride, lithium aluminum deuteride, diisobutylaluminum hydride, n-butyl-diisobutylaluminum hydride, sodium bis-methoxyethoxy, aluminum hydride, triethoxysilane, diethoxymethylsilane, lithium hydride, lithium, sodium, hydrogen Ni/B, and the like. Certain acidic and Lewis acidic reagents enhance the activity of reducing reagents. Examples of such acidic reagents include: acetic acid, methanesulfonic acid, hydrochloric acid, and the like. Examples of such Lewis acidic reagents include: trimethoxyborane, triethoxyborane, aluminum trichloride, lithium chloride, vanadium trichloride, dicyclopentadienyl titanium dichloride, cesium fluoride, potassium fluoride, zinc (II) chloride, zinc (II) bromide, zinc (II) iodide, and the like.

The term “alkylating reagent” refers to any electrophillic reagent capable of transferring an unsubstituted or substituted alkyl group to a nucleophile and as such would be obvious to one of ordinary skill and knowledge in the art. Alkylating reagents include, but are not limited to, compounds having the structure R₁₀₀-LG, where R₁₀₀ is an alkyl group and LG is a leaving group. Specific examples of alkylating reagents include iodomethane, dimethyl sulfate, dimethyl carbonate, methyl toluenesulfonate, and methyl methanesulfonate.

The term “leaving group” (LG) refers to any atom (or group of atoms) that is stable in its anion or neutral form after it has been displaced by a nucleophile and as such would be obvious to one of ordinary skill and knowledge in the art. The definition of “leaving group” includes but is not limited to: water, methanol, ethanol, chloride, bromide, iodide, an alkylsulfonate, for example methanesulfonate, ethanesulfonate and the like, an arylsulfonate, for example benzenesulfonate, tolylsulfonate and the like, a perhaloalkanesulfonate, for example trifluoromethanesulfonate, trichloromethanesulfonate and the like, an alkylcarboxylate, for example acetate and the like, a perhaloalkylcarboxylate, for example trifluoroacetate, trichloroacetate and the like, an arylcarboxylate, for example benzoate and the like.

The terms “alkyl” and “substituted alkyl” are interchangeable and include substituted, optionally substituted and unsubstituted C₁-C₁₀ straight chain saturated aliphatic hydrocarbon groups, substituted, optionally substituted and unsubstituted C₂-C₁₀ straight chain unsaturated aliphatic hydrocarbon groups, substituted, optionally substituted and unsubstituted C₂-C₁₀ branched saturated aliphatic hydrocarbon groups, substituted and unsubstituted C₂-C₁₀ branched unsaturated aliphatic hydrocarbon groups, substituted, optionally substituted and unsubstituted C₃-C₈ cyclic saturated aliphatic hydrocarbon groups, substituted, optionally substituted and unsubstituted C₅-C₈ cyclic unsaturated aliphatic hydrocarbon groups having the specified number of carbon atoms. For example, the definition of “alkyl” shall include but is not limited to: methyl (Me), trideuteromethyl (—CD₃), ethyl (Et), propyl (Pr), butyl (Bu), pentyl, hexyl, heptyl, octyl, nonyl, decyl, undecyl, ethenyl, propenyl, butenyl, penentyl, hexenyl, heptenyl, octenyl, nonenyl, decenyl, undecenyl, isopropyl (i-Pr), isobutyl (i-Bu), tert-butyl (t-Bu), sec-butyl (s-Bu), isopentyl, neopentyl, cyclopropyl, cyclobutyl, cyclopentyl, cyclohexyl, cycloheptyl, cyclooctyl, cyclopentenyl, cyclohexenyl, cycloheptenyl, cyclooctenyl, methylcyclopropyl, ethylcyclohexenyl, butenylcyclopentyl, adamantyl, norbornyl and the like. Alkyl substituents are independently selected from the group consisting of hydrogen, deuterium, halogen, —OH, —SH, —NH₂, —CN, —NO₂, ═O, ═CH₂, trihalomethyl, carbamoyl, arylC₀₋₁₀alkyl, heteroarylC₀₋₁₀alkyl, C₁₋₁₀alkyloxy, arylC₀₋₁₀alkyloxy, C₁₋₁₀alkylthio, arylC₀₋₁₀alkylthio, C₁₋₁₀alkylamino, arylC₀₋₁₀alkylamino, N-aryl-N—C₀₋₁₀alkylamino, C₁₋₁₀alkylcarbonyl, arylC₀₋₁₀alkylcarbonyl, C₁₋₁₀alkylcarboxy, arylC₀₋₁₀alkylcarboxy, C₁₋₁₀alkylcarbonylamino, arylC₀₋₁₀alkylcarbonylamino, tetrahydrofuryl, morpholinyl, piperazinyl, hydroxypyronyl, —C₀₋₁₀alkylCOOR₁₀₁ and —C₀₋₁₀alkylCONR₁₀₂R₁₀₃ wherein R₁₀₁, R₁₀₂ and R₁₀₃ are independently selected from the group consisting of hydrogen, deuterium, alkyl, aryl, or R₃₂ and R₃₃ are taken together with the nitrogen to which they are attached forming a saturated cyclic or unsaturated cyclic system containing 3 to 8 carbon atoms with at least one substituent as defined herein.

The term “aryl” represents an unsubstituted, mono-, or polysubstituted monocyclic, polycyclic, biaryl aromatic groups covalently attached at any ring position capable of forming a stable covalent bond, certain preferred points of attachment being apparent to those skilled in the art (e.g., 3-phenyl, 4-naphthyl and the like). The aryl substituents are independently selected from the group consisting of hydrogen, deuterium, halogen, —OH, —SH, —CN, —NO₂, trihalomethyl, hydroxypyronyl, C₁₋₁₀alkyl, arylC₀₋₁₀alkyl, C₀₋₁₀alkyloxyC₀₋₁₀alkyl, arylC₀₋₁₀alkyloxyC₀₋₁₀alkyl, C₀₋₁₀alkylthioC₀₋₁₀alkyl, arylC₀₋₁₀alkylthioC₀₋₁₀alkyl, C₀₋₁₀alkylaminoC₀₋₁₀alkyl, arylC₀₋₁₀alkylaminoC₀₋₁₀alkyl, N-aryl-N—C₀₋₁₀alkylaminoC₀₋₁₀alkyl, C₁₋₁₀alkylcarbonylC₀₋₁₀alkyl, arylC₀₋₁₀alkylcarbonylC₀₋₁₀alkyl, C₁₋₁₀alkylcarboxyC₀₋₁₀alkyl, arylC₀₋₁₀alkylcarboxyC₀₋₁₀alkyl, C₁₋₁₀alkylcarbonylaminoC₀₋₁₀alkyl, arylC₀₋₁₀alkylcarbonylaminoC₀₋₁₀alkyl, —C₀₋₁₀alkylCOOR₁₀₁ and —C₀₋₁₀alkylCONR₁₀₂R₁₀₃ wherein R₁₀₁, R₁₀₂ and R₁₀₃ are independently selected from the group consisting of hydrogen, deuterium, alkyl, aryl or R₃₁ and R₃₂ are taken together with the nitrogen to which they are attached forming a saturated cyclic or unsaturated cyclic system containing 3 to 8 carbon atoms with at least one substituent as defined above.

The definition of “aryl” includes but is not limited to phenyl, pentadeuterophenyl, biphenyl, naphthyl, dihydronaphthyl, tetrahydronaphthyl, indenyl, indanyl, azulenyl, anthryl, phenanthryl, fluorenyl, pyrenyl and the like.

The term “electron-transfer reagent” refers to an agent capable of transferring a single electron between an alkali metal and thiophene. Electron-transfer reagents are generally aliphatic or aromatic conjugated diene compounds that may be selected from the compounds of formula R₁₀₈C(R₁₀₆)═CHR₁₀₇, in which R₁₀₆ is hydrogen, methyl, ethyl, or phenyl; R₁₀₇ is hydrogen or a methyl or ethyl radical; R₁₀₈ is phenyl or R₁₀₉CH═C(R₁₁₀)—, in which R₁₀₉ and R₁₁₀ are independently hydrogen, methyl, or ethyl. Examples of electron-transfer reagents include, but are not limited to, 1,3-butadiene, 2-methyl-1,3-butadiene (isoprene), 1-phenylethylene (styrene), 1-methyl-1phenylethylene (α-methylstyrene), 1,1-phenylethylene, 2,3-dimethyl-1,3-butadiene, 1,3-pentadiene, and 2,4-hexadiene.

The compounds disclosed herein can exist as therapeutically acceptable salts. The term “therapeutically acceptable salt,” as used herein, represents salts or zwitterionic forms of the compounds disclosed herein which are therapeutically acceptable as defined herein. The salts can be prepared during the final isolation and purification of the compounds or separately by reacting the appropriate compound with a suitable acid or base. Therapeutically acceptable salts include acid and basic addition salts. For a more complete discussion of the preparation and selection of salts, refer to “Handbook of Pharmaceutical Salts, Properties, and Use,” Stah and Wermuth, Ed.; (Wiley-VCH and VHCA, Zurich, 2002) and Berge et al., J. Pharm. Sci. 1977, 66, 1-19.

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

While it may be possible for the compounds of the subject invention to be administered as the raw chemical, it is also possible to present them as a pharmaceutical composition. Accordingly, provided herein are pharmaceutical compositions which comprise one or more of certain compounds disclosed herein, or one or more pharmaceutically acceptable salts, prodrugs, or solvates thereof, together with one or more pharmaceutically acceptable carriers thereof and optionally one or more other therapeutic ingredients. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Deliver Technology, Rathbone et al., Eds., Drugs and the Pharmaceutical Science, Marcel Dekker, Inc.: New York, N.Y., 2002; Vol. 126).

The compositions include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration. The most suitable route for administration depends on a variety of factors, including interpatient variation or disorder type, and therefore the invention is not limited to just one form of administration. The compositions may conveniently be presented in unit dosage form and may be prepared by any of the methods well known in the art of pharmacy. Typically, these methods include the step of bringing into association a compound of the subject invention or a pharmaceutically salt, prodrug, or solvate thereof (“active ingredient”) with the carrier which constitutes one or more accessory ingredients. In general, the compositions are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose.

For administration by inhalation, compounds may be delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the disorder being treated. Also, the route of administration may vary depending on the disorder and its severity.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disorder.

In the case wherein the patient's status does improve, upon the doctor's discretion the administration of the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

Disclosed herein are methods of treating an opioid receptor-mediated disorder comprising administering to a subject having or suspected to have such a disorder, a therapeutically effective amount of a compound as disclosed herein or a pharmaceutically acceptable salt, solvate, or prodrug thereof.

Opioid receptor-mediated disorders, include, but are not limited to, preoperative pain, intraoperative pain, postoperative pain, partuition, oncological pain, neuropathy, osteoarthritis, rheumatoid arthritis, fibromyalgia, pancreatitis, herniated discs, migraine, trigeminal neuralgia, sciatica, myocardial infarctions, any procedure which requires anesthesia or sedation, any disorder associated with moderate to severe acute or chronic pain, and/or any disorder ameliorated by the modulation of opioid receptors.

In certain embodiments, a method of treating an opioid receptor-mediated disorder comprises administering to the subject a therapeutically effective amount of a compound of as disclosed herein, or a pharmaceutically acceptable salt, solvate, or prodrug thereof, so as to affect: (1) decreased inter-individual variation in plasma levels of the compound or a metabolite thereof; (2) increased average plasma levels of the compound or decreased average plasma levels of at least one metabolite of the compound per dosage unit; (3) decreased inhibition of, and/or metabolism by at least one cytochrome P₄₅₀ or monoamine oxidase isoform in the subject; (4) decreased metabolism via at least one polymorphically-expressed cytochrome P₄₅₀ isoform in the subject; (5) at least one statistically-significantly improved disorder-control and/or disorder-eradication endpoint; (6) an improved clinical effect during the treatment of the disorder; (7) prevention of recurrence, or delay of decline or appearance, of abnormal alimentary or hepatic parameters as the primary clinical benefit; or (8) reduction or elimination of deleterious changes in any diagnostic hepatobiliary function endpoints, as compared to the corresponding non-isotopically enriched compound.

In certain embodiments, inter-individual variation in plasma levels of the compounds as disclosed herein, or metabolites thereof, is decreased; average plasma levels of the compound as disclosed herein are increased; average plasma levels of a metabolite of the compound as disclosed herein are decreased; inhibition of a cytochrome P₄₅₀ or monoamine oxidase isoform by a compound as disclosed herein is decreased; or metabolism of the compound as disclosed herein by at least one polymorphically-expressed cytochrome P₄₅₀ isoform is decreased; by greater than about 5%, greater than about 10%, greater than about 20%, greater than about 30%, greater than about 40%, or by greater than about 50% as compared to the corresponding non-isotopically enriched compound.

Plasma levels of the compound as disclosed herein, or metabolites thereof, may be measured using the methods described by Li et al. Rapid Communications in Mass Spectrometry 2005, 19, 1943-1950, Geyer et al., European Journal of Mass Spectrometry 2005, 11(4), 419-427, Huynh et al., Journal of Pharmaceutical and Biomedical Analysis 2005, 37(5), 1095-1100, Van Nimmen et al., Journal of Chromatography, B: Analytical Technologies in the Biomedical and Life Sciences 2004, 804(2), 375-387, Sachs et al., International Journal of Legal Medicine 1996, 109(4), 213-5, Mautz et al., Journal of chromatography. B, Biomedical applications 1994, 658(1), 149-53, and references cited therein and any routine modifications thereof.

Examples of cytochrome P₄₅₀ isoforms in a mammalian subject include, but are not limited to, CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51.

Examples of monoamine oxidase isoforms in a mammalian subject include, but are not limited to, MAO_{A}, and MAO_{B}.

The inhibition of the cytochrome P₄₅₀ isoform is measured by the method of Ko et al. (British Journal of Clinical Pharmacology, 2000, 49, 343-35 1). The inhibition of the MAO_{A} isoform is measured by the method of Weyler et al. (J. Biol Chem. 1985, 260, 13199-13207). The inhibition of the MAO_{B} isoform is measured by the method of Uebelhack et al. (Pharmacopsychiatry, 1998, 31, 187-192).

Examples of polymorphically-expressed cytochrome P₄₅₀ isoforms in a mammalian subject include, but are not limited to, CYP2C8, CYP2C9, CYP2C19, and CYP2D6.

The metabolic activities of liver microsomes, cytochrome P₄₅₀ isoforms, and monoamine oxidase isoforms are measured by the methods described herein.

Examples of improved disorder-control and/or disorder-eradication endpoints, or improved clinical effects include, but are not limited to, statistically-significant improvement in pain indices, prevention of irreversible arterial hypotension, multiple organ dysfunction syndrome, decreased mortality, normalization of heart rate, normalization of body temperature, normalization of blood gases, normalization of white blood cell count, reduction in need for hemodialysis, and/or diminution of toxicity including but not limited to, hepatotoxicity or other toxicity, or a decrease in aberrant liver enzyme levels as measured by standard laboratory protocols, as compared to the corresponding non-isotopically enriched compound when given under the same dosing protocol including the same number of doses per day and the same quantity of drug per dose.

Examples of diagnostic hepatobiliary function endpoints include, but are not limited to, alanine aminotransferase (“ALT”), serum glutamic-pyruvic transaminase (“SGPT”), aspartate aminotransferase (“AST” or “SGOT”), ALT/AST ratios, serum aldolase, alkaline phosphatase (“ALP”), ammonia levels, bilirubin, gamma-glutamyl transpeptidase (“GGTP,” “γ-GTP,” or “GGT”), leucine aminopeptidase (“LAP”), liver biopsy, liver ultrasonography, liver nuclear scan, 5′-nucleotidase, and blood protein. Hepatobiliary endpoints are compared to the stated normal levels as given in “Diagnostic and Laboratory Test Reference”, 4^{th} edition, Mosby, 1999. These assays are run by accredited laboratories according to standard protocol.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

### Combination Therapy

The compounds disclosed herein may also be combined or used in combination with other agents useful in the treatment of opioid receptor-mediated disorders. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced).

Such other agents, adjuvants, or drugs, may be administered, by a route and in an amount commonly used therefor, simultaneously or sequentially with a compound as disclosed herein. When a compound as disclosed herein is used contemporaneously with one or more other drugs, a pharmaceutical composition containing such other drugs in addition to the compound disclosed herein may be utilized, but is not required.

In certain embodiments, the compounds provided herein can be combined with one or more opioids known in the art, including, but not limited to, morphine, codeine, thebain, diacetylmorphine, oxycodone, hydrocodone, hydromorphone, oxymorphone, nicomorphine, fentanyl, α-methylfentanyl, alfentanil, sufentanil, remifentanyl, carfentanyl, ohmefentanyl, pethidine, ketobemidone, propoxyphene, dextropropoxyphene, methadone, loperamide, pentazocine, buprenorphine, etorphine, butorphanol, nalbufine, levorphanol, naloxone, naltrexone, and tramadol.

In certain embodiments, the compounds provided herein can be combined with one or more local or general anesthetics known in the art, including, but not limited to, diethyl ether, vinyl ether, halothane, chloroform, methoxyflurane, enflurane, trichloroethylene, isoflurane, desflurane, sevoflurane, methohexital, hexobarbital, thiopental, narcobarbital, fentanyl, alfentanil, sufentanil, phenoperidine, anileridine, remifentanil, droperidol, ketamine, propanidid, alfaxalone, etomidate, propofol, hydroxybutyric acid, nitrous oxide, esketamine, metabutethamine, procaine, tetracaine, chloroprocaine, benzocaine, bupivacaine, lidocaine, mepivacaine, prilocaine, butanilicaine, cinchocaine, etidocaine, articaine, ropivacaine, levobupivacaine, cocaine, ethyl chloride, dyclonine, phenol, and capsaicin.

In certain embodiments, the compounds provided herein can be combined with one or more steroidal drugs known in the art, including, but not limited to, aldosterone, beclometasone, betamethasone, deoxycorticosterone acetate, fludrocortisone acetate, hydrocortisone (cortisol), prednisolone, prednisone, methylprenisolone, dexamethasone, and triamcinolone.

In certain embodiments, the compounds provided herein can be combined with one or more antibacterial agents known in the art, including, but not limited to, amikacin, amoxicillin, ampicillin, arsphenamine, azithromycin, aztreonam, azlocillin, bacitracin, carbenicillin, cefaclor, cefadroxil, cefamandole, cefazolin, cephalexin, cefdinir, cefditorin, cefepime, cefixime, cefoperazone, cefotaxime, cefoxitin, cefpodoxime, cefprozil, ceftazidime, ceftibuten, ceftizoxime, ceftriaxone, cefuroxime, chloramphenicol, cilastin, ciprofloxacin, clarithromycin, clindamycin, cloxacillin, colistin, dalfopristan, demeclocycline, dicloxacillin, dirithromycin, doxycycline, erythromycin, enafloxacin, ertepenem, ethambutol, flucloxacillin, fosfomycin, furazolidone, gatifloxacin, geldanamycin, gentamicin, herbimicin, imipenem, isoniazide, kanamicin, levofloxacin, linezolid, lomefloxacin, loracarbef, mafenide, moxifloxacin, meropenem, metronidazole, mezlocillin, minocycline, mupirozin, nafcillin, neomycin, netilmicin, nitrofurantoin, norfloxacin, ofloxacin, oxytetracycline, penicillin, piperacillin, platensimycin, polymixin B, prontocil, pyrazinamide, quinupristine, rifampin, roxithromycin, spectinomycin, streptomycin, sulfacetamide, sulfamethizole, sulfamethoxazole, teicoplanin, telithromycin, tetracycline, ticarcillin, tobramycin, trimethoprim, troleandomycin, trovafloxacin, and vancomycin.

In certain embodiments, the compounds provided herein can be combined with one or more sepsis treatments known in the art, including, but not limited to drotrecogin-α or a biosimilar of activated protein C.

In certain embodiments, the compounds provided herein can be combined with one or more antifungal agents known in the art, including, but not limited to, amorolfine, amphotericin B, anidulafungin, bifonazole, butenafine, butoconazole, caspofungin, ciclopirox, clotrimazole, econazole, fenticonazole, filipin, fluconazole, isoconazole, itraconazole, ketoconazole, micafungin, miconazole, naftifine, natamycin, nystatin, oxyconazole, ravuconazole, posaconazole, rimocidin, sertaconazole, sulconazole, terbinafine, terconazole, tioconazole, and voriconazole.

In certain embodiments, the compounds provided herein can be combined with one or more anticoagulants known in the art, including, but not limited to, acenocoumarol, argatroban, bivalirudin, lepirudin, fondaparinux, heparin, phenindione, warfarin, and ximalagatran.

In certain embodiments, the compounds provided herein can be combined with one or more thrombolytics known in the art, including, but not limited to, anistreplase, reteplase, t-PA (alteplase activase), streptokinase, tenecteplase, and urokinase.

In certain embodiments, the compounds provided herein can be combined with one or more non-steroidal anti-inflammatory agents known in the art, including, but not limited to, aceclofenac, acemetacin, amoxiprin, aspirin, azapropazone, benorilate, bromfenac, carprofen, celecoxib, choline magnesium salicylate, diclofenac, diflunisal, etodolac, etoracoxib, faislamine, fenbuten, fenoprofen, flurbiprofen, ibuprofen, indometacin, ketoprofen, ketorolac, lornoxicam, loxoprofen, lumiracoxib, meclofenamic acid, mefenamic acid, meloxicam, metamizole, methyl salicylate, magnesium salicylate, nabumetone, naproxen, nimesulide, oxyphenbutazone, parecoxib, phenylbutazone, piroxicam, salicyl salicylate, sulindac, sulfinprazone, suprofen, tenoxicam, tiaprofenic acid, and tolmetin.

In certain embodiments, the compounds provided herein can be combined with one or more antiplatelet agents known in the art, including, but not limited to, abciximab, cilostazol, clopidogrel, dipyridamole, ticlopidine, and tirofibin.

The compounds disclosed herein can also be administered in combination with other classes of compounds, including, but not limited to, norepinephrine reuptake inhibitors (NRIs) such as atomoxetine; dopamine reuptake inhibitors (DARIs), such as methylphenidate; serotonin-norepinephrine reuptake inhibitors (SNRIs), such as milnacipran; sedatives, such as diazepham; norepinephrine-dopamine reuptake inhibitor (NDRIs), such as bupropion; serotonin-norepinephrine-dopamine-reuptake-inhibitors (SNDRIs), such as venlafaxine; monoamine oxidase inhibitors, such as selegiline; hypothalamic phospholipids; endothelin converting enzyme (ECE) inhibitors, such as phosphoramidon; thromboxane receptor antagonists, such as ifetroban; potassium channel openers; thrombin inhibitors, such as hirudin; hypothalamic phospholipids; growth factor inhibitors, such as modulators of PDGF activity; platelet activating factor (PAF) antagonists; low molecular weight heparins, such as enoxaparin; Factor VIIa Inhibitors and Factor Xa Inhibitors; renin inhibitors; neutral endopeptidase (NEP) inhibitors; vasopepsidase inhibitors (dual NEP-ACE inhibitors), such as omapatrilat and gemopatrilat; HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, or atavastatin or visastatin); squalene synthetase inhibitors; fibrates; bile acid sequestrants, such as questran; niacin; anti-atherosclerotic agents, such as ACAT inhibitors; MTP Inhibitors; calcium channel blockers, such as amlodipine besylate; potassium channel activators; alpha-muscarinic agents; beta-muscarinic agents, such as carvedilol and metoprolol; antiarrhythmic agents; diuretics, such as chlorothiazide, hydrochlorothiazide, flumethiazide, hydroflumethiazide, bendroflumethiazide, methylchlorothiazide, trichloromethiazide, polythiazide, benzothiazide, ethacrynic acid, tricrynafen, chlorthalidone, furosenilde, musolimine, bumetanide, triamterene, amiloride, and spironolactone; anti-diabetic agents, such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists, such as spironolactone and eplerenone; growth hormone secretagogues; aP2 inhibitors; phosphodiesterase inhibitors, such as PDE III inhibitors (e.g., cilostazol) and PDE V inhibitors (e.g., sildenafil, tadalafil, vardenafil); protein tyrosine kinase inhibitors; antiproliferatives, such as methotrexate, FK506 (tacrolimus, Prograf), mycophenolate mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes); antimetabolites, such as folate antagonists, purine analogues, and pyrridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, and octreotide acetate; microtubule-disruptor agents, such as ecteinascidins; microtubule-stabilizing agents, such as pacitaxel, docetaxel, and epothilones A-F; plant-derived products, such as vinca alkaloids, epipodophyllotoxins, and taxanes; and topoisomerase inhibitors; prenyl-protein transferase inhibitors; and cyclosporins; cytotoxic drugs, such as azathiprine and cyclophosphamide; TNF-alpha inhibitors, such as tenidap; anti-TNF antibodies or soluble TNF receptor, such as etanercept, rapamycin, and leflunimide; and cyclooxygenase-2 (COX-2) inhibitors, such as celecoxib and rofecoxib; and miscellaneous agents such as, hydroxyurea, procarbazine, mitotane, hexamethylmelamine, gold compounds, platinum coordination complexes, such as cisplatin, satraplatin, and carboplatin.

Thus, in another aspect, certain embodiments provide methods for treating opioid receptor-mediated disorders in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of opioid receptor-mediated disorders.

### General Synthetic Methods for Preparing Compounds

Isotopic hydrogen can be introduced into a compound as disclosed herein by synthetic techniques that employ deuterated reagents, whereby incorporation rates are pre-determined; and/or by exchange techniques, wherein incorporation rates are determined by equilibrium conditions, and may be highly variable depending on the reaction conditions. Synthetic techniques, where tritium or deuterium is directly and specifically inserted by tritiated or deuterated reagents of known isotopic content, may yield high tritium or deuterium abundance, but can be limited by the chemistry required. Exchange techniques, on the other hand, may yield lower tritium or deuterium incorporation, often with the isotope being distributed over many sites on the molecule.

The compounds as disclosed herein can be prepared by methods known to one of skill in the art and routine modifications thereof, and/or following procedures similar to those described in the Example section herein and routine modifications thereof, and/or procedures found in Lee et al., Bioorganic & Medicinal Chemistry Letters 17 2007, 2161-2165, Colapret et al., Journal of Medicinal Chemistry 1989, 32, 968-974, Janssens et al., Journal of Medicinal Chemistry 1986, 29(11), 2290-7, WO 2001/40184, U.S. Pat. No. 6,639,083, WO 2008/005423, and U.S. Pat. No. 3,164,600, and references cited therein and routine modifications thereof. Compounds as disclosed herein can also be prepared as shown in any of the following schemes and routine modifications thereof.

The following schemes can be used to practice the present invention.

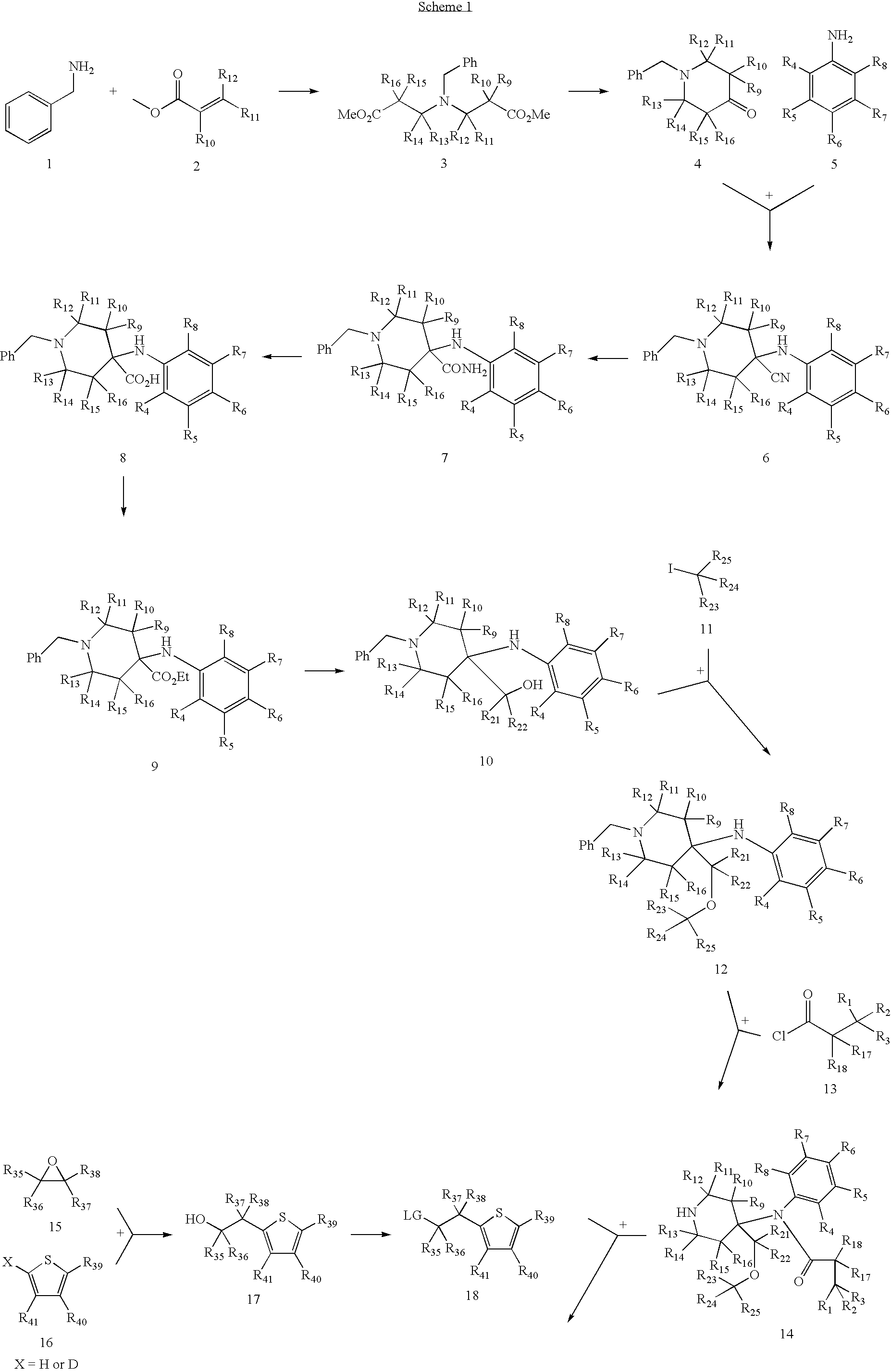

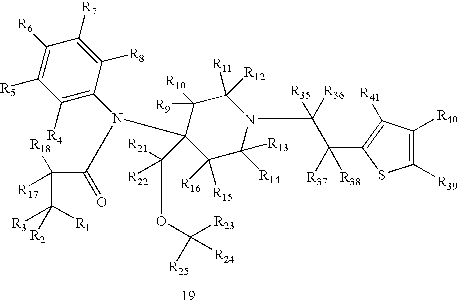

Compound 1 is reacted with compound 2 in an appropriate solvent, such as methanol, at an elevated temperature, to give compound 3. Compound 3 is treated with an appropriate base, such as sodium, in an appropriate solvent, such as toluene, at an elevated temperature to give compound 4. Compound 4 is reacted with compound 5 in the presence of an appropriate cyanide salt, such as potassium cyanide, at an elevated temperature, to give compound 6. Compound 6 is treated with an appropriate acid, such as sulfuric acid, in an appropriate solvent, such as water, to afford compound 7. Compound 7 is treated with an appropriate base, such as potassium hydroxide, in an appropriate solvent, such as 1,2-propanediol, at an elevated temperature to give compound 8. Compound 8 is reacted with an appropriate alcohol, such as ethanol, in an appropriate solvent, such as toluene, at an elevated temperature, to afford compound 9. Compound 9 is treated with an appropriate reducing reagent, such as lithium aluminum hydride, in an appropriate solvent, such as tetrahydrofuran, at an elevated temperature, to give compound 10. Compound 10 is treated with an appropriate base, such as sodium hydride, in an appropriate solvent, such as tetrahydrofuran, and then reacted with compound 11, to give compound 12. Compound 12 is reacted with compound 13 in an appropriate solvent, such as dichloromethane, in the presence of an appropriate base, such triethylamine, to give an intermediate amide, which is then treated with an appropriate reducing reagent, such as a combination of hydrogen gas and an appropriate catalyst, such as palladium on carbon, in an appropriate solvent, such as a combination of water and acetic acid, at an elevated temperature, to give compound 14. Compound 15 is reacted with compound 16 in the presence of an appropriate base, such as sodium, and an appropriate electron-transfer reagent, such as 2,3-dimethyl-1,3-butadiene, in an appropriate solvent, such as a combination of tetrahydrofuran and toluene, to give compound 17. Compound 17 is reacted with an appropriate leaving group reagent (LG), such as methanesulfonyl chloride, in the presence of an appropriate base, such as triethylamine, in an appropriate solvent, such as methylene chloride, to give compound 18. Compound 18 is reacted with compound 14 in the presence of an appropriate base, such as triethylamine, in an appropriate solvent, such as acetonitrile, at an elevated temperature to afford compound 19 of Formula I.

Deuterium can be incorporated to different positions synthetically, according to the synthetic procedures as shown in Scheme 1, by using appropriate deuterated intermediates. For example, to introduce deuterium at one or more positions of R₁₀, R₁₁, R₁₂, R₁₃, R₁₄, and R₁₅, compound 2 with the corresponding deuterium substitutions can be used. To introduce deuterium at one or more positions of R₉ and R₁₆, methanol with the corresponding deuterium substitutions can be used. To introduce deuterium at one or more positions of R₄, R₅, R₆, R₇, and R₈, compound 5 with the corresponding deuterium substitutions can be used. To introduce deuterium at one or more positions of R₂₁ and R₂₂, lithium aluminum deuteride can be used. To introduce deuterium at one or more positions of R₂₃, R₂₄, and R₂₅, compound 11 with the corresponding deuterium substitutions can be used. To introduce deuterium at one or more positions of R₁, R₂, R₃, R₁₇, and R₁₈, compound 13 with the corresponding deuterium substitutions can be used. To introduce deuterium at position R₃₅, R₃₆, R₃₇, and R₃₈, compound 15 with the corresponding deuterium substitutions can be used. To introduce deuterium at position R₃₉, R₄₀, and R₄₁, compound 16 with the corresponding deuterium substitutions can be used. These deuterated intermediates are either commercially available, or can be prepared by methods known to one of skill in the art or following procedures similar to those described in the Example section herein and routine modifications thereof.

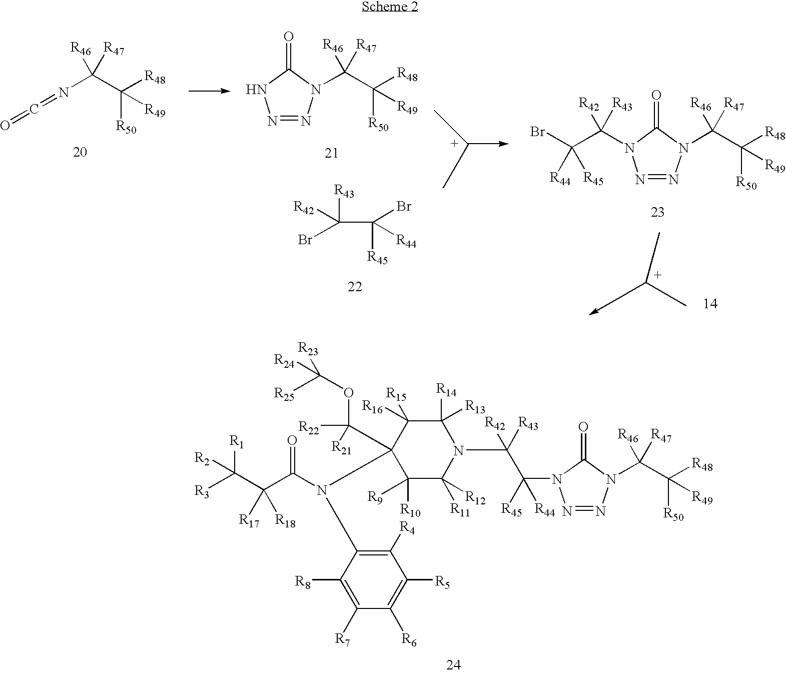

Compound 20 is reacted with sodium azide, in the presence of an appropriate catalyst, such as aluminum chloride, in an appropriate solvent, such as tetrahydrofuran, at an elevated temperature to give compound 21. Compound 21 is reacted with compound 22 in the presence of an appropriate base, such as triethylamine, an in an appropriate solvent, such as acetonitrile, at an elevated temperature, to give compound 23. Compound 23 is reacted with compound 14 in the presence of an appropriate base, such as sodium carbonate, and an appropriate catalyst, such as potassium iodide, in an appropriate solvent, such as water, at an elevated temperature to give compound 24 of Formula I.

Deuterium can be incorporated to different positions synthetically, according to the synthetic procedures as shown in Scheme 2, by using appropriate deuterated intermediates. For example, to introduce deuterium at one or more positions of R₄₆, R₄₇, R₄₈, R₄₉, and R₅₀, compound 20 with the corresponding deuterium substitutions can be used. To introduce deuterium at one or more positions of R₄₂, R₄₃, R₄₄, and R₄₅, compound 22 with the corresponding deuterium substitutions can be used. To introduce deuterium at one or more positions of R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₂₁, R₂₂, R₂₃, R₂₄, and R₂₅, compound 14 with the corresponding deuterium substitutions can be used. These deuterated intermediates are either commercially available, or can be prepared by methods known to one of skill in the art or following procedures similar to those described in the Example section herein and routine modifications thereof.

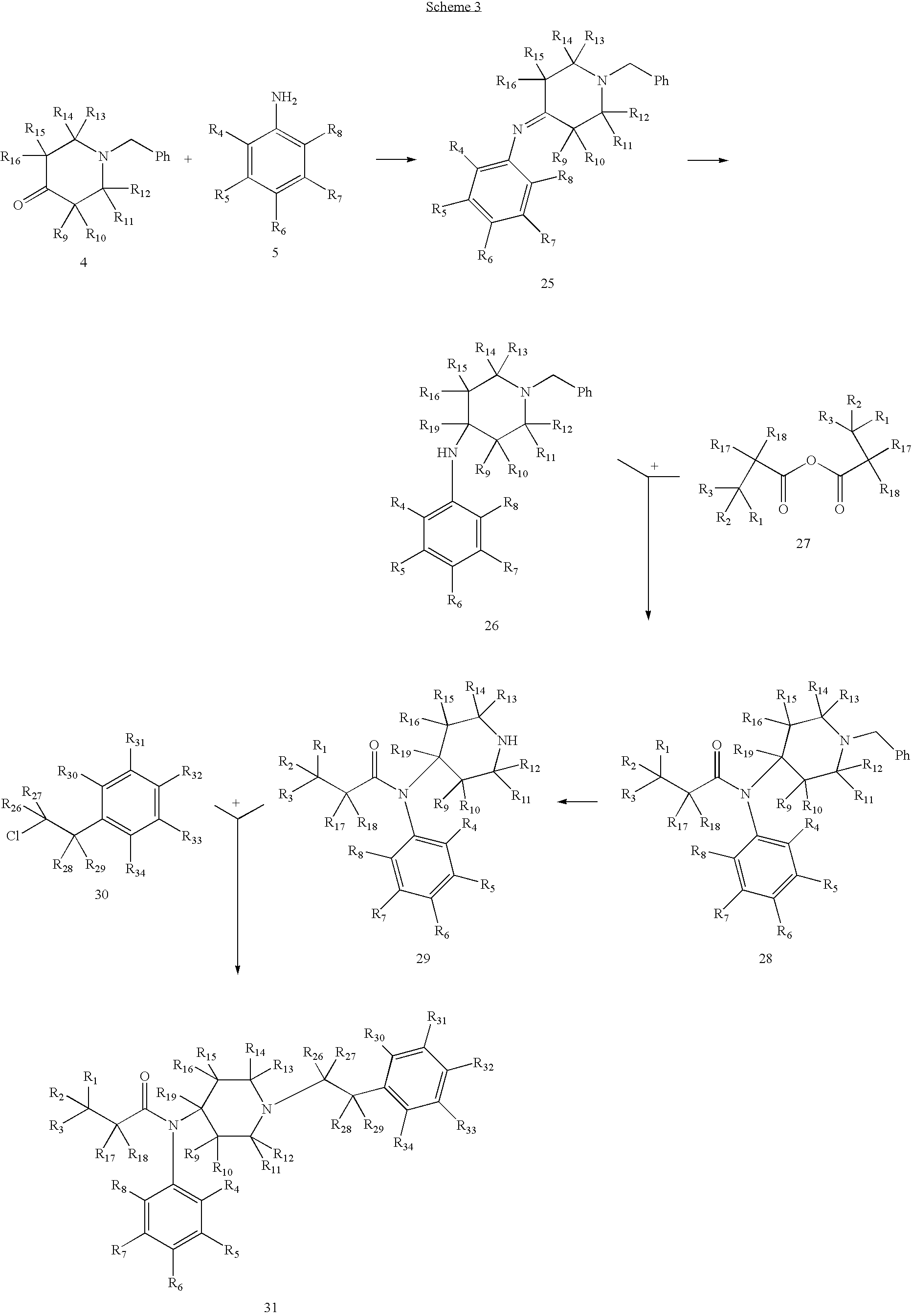

Compound 4 is reacted with compound 5 in the presence of an appropriate acid, such as toluenesulfonic acid, in an appropriate solvent, such as toluene, at an elevated temperature to give compound 25. Compound 25 is treated with an appropriate reducing reagent, such as lithium aluminum hydride, in an appropriate solvent, such as ether, at an elevated temperature, to give compound 26. Compound 26 is reacted with compound 27 in an appropriate solvent, such as toluene, at an elevated temperature, to afford compound 28. Compound 28 is treated with an appropriate reducing reagent, such as a combination of hydrogen gas and an appropriate catalyst, such as palladium on carbon, in an appropriate solvent, such as ethanol, to give compound 29. Compound 29 is reacted with compound 30 in the presence of an appropriate base, such as sodium carbonate, an appropriate catalyst, such as potassium iodide, in an appropriate solvent, such as 4-methyl-2-pentanone, at an elevated temperature, to afford compound 31 of Formula I.

Deuterium can be incorporated to different positions synthetically, according to the synthetic procedures as shown in Scheme 1, by using appropriate deuterated intermediates. For example, to introduce deuterium at one or more positions of R₉, R₁₀, R₁₁, R₁₂, R₁₃, R₁₄, R₁₅, and R₁₆, compound 4 with the corresponding deuterium substitutions can be used. To introduce deuterium at one or more positions of R₄, R₅, R₆, R₇, and R₈, compound 5 with the corresponding deuterium substitutions can be used. To introduce deuterium at position R₁₉, lithium aluminum deuteride can be used. To introduce deuterium at one or more positions of R₁, R₂, R₃, R₁₇, and R₁₈, compound 27 with the corresponding deuterium substitutions can be used. To introduce deuterium at one or more positions of R₂₆, R₂₇, R₂₈, R₂₉, R₃₀, R₃₁, R₃₂, R₃₃, and R₃₄, compound 30 with the corresponding deuterium substitutions can be used. These deuterated intermediates are either commercially available, or can be prepared by methods known to one of skill in the art or following procedures similar to those described in the Example section herein and routine modifications thereof.

The invention is further illustrated by the following examples. All IUPAC names were generated using CambridgeSoft's ChemDraw 10.0.

### EXAMPLE 1

### N-(1-phenethylpiperidin-4-yl)-N-phenylpropionamide citrate

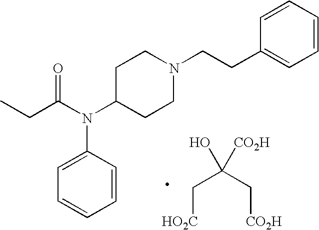

### Step 1

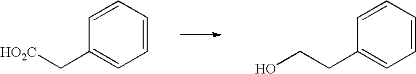

2-Phenylethanol: At about 0° C., a solution of phenylacetic acid (5.0 g; 36.72 mmol) in tetrahydrofuran (30 mL) was added dropwise to a suspension of lithium aluminum hydride (2.1 g; 55.34 mmol) in tetrahydrofuran (20 mL). The mixture was stirred at ambient temperature for about 3 hours, and then cooled to about 0° C. After cold water (5 mL) was added, the mixture was filtered and the inorganic salts washed with ethyl acetate. The filtrate and washings were combined and concentrated in vacuo to give the title compound as a colorless liquid (4.0 g; 89%). ¹H NMR (400 MHz, CDCl₃) δ 1.45 (br, exchangeable with D₂O, 1H), 2.87 (t, J=6.6 Hz, 2H), 3.86 (t, J=6.6 Hz, 2H), 7.18-7.35 (m, 5H); IR (film) υ 3363, 3028, 2940, 2875, 1495, 1453 cm⁻¹.

### Step 2

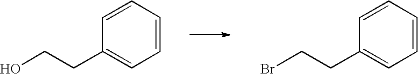

(2-Bromoethyl)benzene: At about 0° C., tetrabromomethane (15.81 g; 47.67 mmol) was added portionwise to a mixture of 2-phenylethanol (4.00 g; 32.74 mmol), triphenylphosphine (14.14 g; 53.91 mmol), dry pyridine (15.40 mL; 190.41 mmol) and dry dichloromethane (30 mL). The mixture was stirred at ambient temperature for about 16 hours, diluted with dichloromethane, and washed with 2N hydrochloric acid and water. The organic layer was concentrated in vacuo to provide a crude residue which was purified by column chromatography on neutral alumina (1-2% ethyl acetate in petroleum ether) to give the title compound as a liquid (5.30 g; 87%). ¹H NMR (400 MHz, CDCl₃) δ 3.17 (t, J=7.6 Hz, 2H), 3.57 (t, J=7.6 Hz, 2H), 7.17-7.36 (m, 5H); IR (film) υ 3026, 2924, 2854, 1495, 1450 cm⁻.

### Step 3

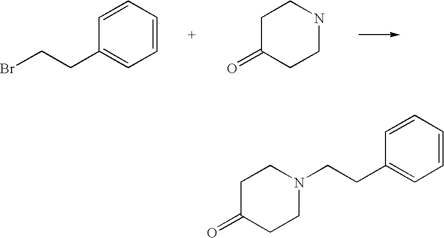

1-Phenethylpiperidin-4-one: A mixture of (2-bromoethyl)benzene (5.30 g; 28.64 mmol), piperidone monohydrate hydrochloride (5.41 g; 31.51 mmol), cesium carbonate (23.33 g; 71.60 mmol) and acetonitrile (40 mL) was heated at reflux for about 6 hours. The reaction mixture was cooled to ambient temperature and filtered. The filtrate was concentrated and the resulting crude residue was purified by column chromatography on neutral alumina (1% ethyl acetate in dichloromethane) to afford the title compound as an oil, which solidified on standing (2.40 g; 41%). ¹H NMR (400 MHz, CDCl₃) δ 2.48 (t, J=6.1 Hz, 4H), 2.68-2.76 (m, 2H), 2.78-2.83 (m, 6H), 7.17-7.33 (m, 5H); IR (film) υ 2954, 2807, 1716 cm⁻¹; MS 204 (M+1).

### Step 4

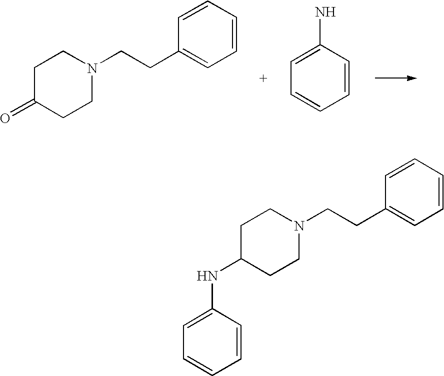

1-Phenethyl-N-phenylpiperidin-4-amine: At about 0° C., aniline (0.45 mL; 4.94 mmol) and sodium triacetoxyborohydride (1.25 g; 5.90 mmol) were added sequentially to a mixture of 1-phenethylpiperidin-4-one (1.00 g; 4.92 mmol), acetic acid (2.0 mL) and dichloromethane (20 mL). The mixture was stirred at ambient temperature for about 24 hours, cooled to about 0° C., and basified to pH 9 with 10% sodium hydroxide. Following standard extractive work up, the crude residue which was purified by column chromatography on neutral alumina (5% ethyl acetate in petroleum ether) to give the title compound as a white solid (0.850 g; 62%). ¹H NMR (400 MHz, CDCl₃) δ 1.44-1.57 (m, 2H), 2.04-2.13 (m, 2H), 2.16-2.27 (m, 2H), 2.58-2.65 (m, 2H), 2.78-2.86 (m, 2H), 2.92-3.01 (m, 2H), 3.26-3.37 (m, 1H), 3.52 (br, exchangeable with D₂O, 1H), 6.60 (d, J=8.0 Hz, 2H), 6.68 (t, J=7.3 Hz, 1H), 7.13-7.32 (m, 7H); IR (film) υ 3399, 3029, 2936, 2805, 1601, 1504, 1450, 1316 cm⁻¹; MS 281 (M+1).

### Step 5

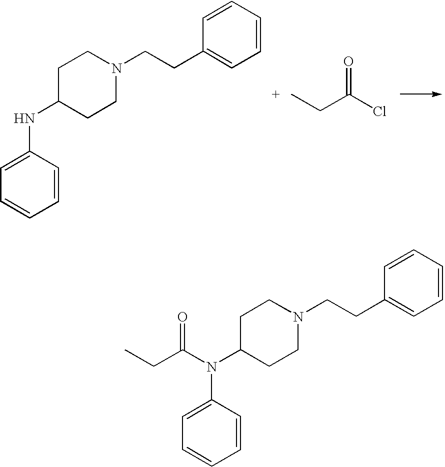

N-(1-Phenethylpiperidin-4-yl)-N-phenylpropionamide: At about 0° C., propionyl chloride (0.07 mL; 0.806 mmol) was added to a mixture of 1-phenethyl-N-phenylpiperidin-4-amine (0.200 g; 0.713 mmol), triethylamine (0.20 mL; 1.435 mmol) and dichloromethane (2 mL). The mixture was stirred at ambient temperature for about 3 hours. Following standard extractive work up, the crude residue was purified by column chromatography on neutral alumina (dichloromethane) to yield the title compound as an oil which solidified on standing (0.200 g; 83%). ¹H NMR (400 MHz, CDCl₃) δ 1.02 (t, J=7.4 Hz, 3H), 1.36-1.48 (m, 2H), 1.76-1.83 (m, 2H), 1.93 (q, J=7.4 Hz, 2H), 2.11-2.21 (m, 2H), 2.49-2.56 (m, 2H), 2.68-2.76 (m, 2H), 2.95-3.03 (m, 2H), 4.63-4.73 (m, 1H), 7.06-7.43 (m, 10H); IR (KBr) υ 2932, 2802, 1650, 1593, 1495, 1452, 1397, 1263 cm⁻¹; MS 337 (M+1).

### Step 6

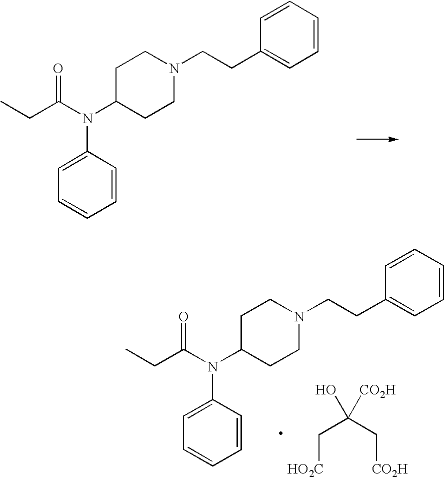

N-(1-Phenethylpiperidin-4-yl)-N-phenylpropionamide citrate: A mixture of N-(1-phenethylpiperidin-4-yl)-N-phenylpropionamide (0.160 g; 0.476 mmol), citric acid monohydrate (0.080 g; 0.381 mmol) and ethanol (1 mL) was heated at reflux for about 4 hours. The clear solution was stirred at ambient temperature for about 16 hours. The resulting precipitate was collected by filteration, washed with cold ethanol, and dried to give the title compound as a white solid (0.155 g; 62%). m.p. 151-152° C.; ¹H NMR (400 MHz, pyridine-d₅) δ 1.10 (t, J=7.4 Hz, 3H), 1.38-1.51 (m, 2H), 1.71-1.80 (m, 2H), 1.61 (q, J=7.4 Hz, 2H), 1.98-2.06 (m, 2H), 2.43-2.52 (m, 2H), 2.68-2.76 (m, 2H), 2.86-2.94 (m, 2H), 3.62 (d, J=14.8 Hz, 2H), 3.71 (d, J=14.8 Hz, 2H), 4.79-4.89 (m, 1H), 7.09-7.44 (m, 10H), 11.20 (br, exchangeable with D₂O, 3H); IR (KBr) υ 3422, 2986, 2945, 1719, 1586, 1429, 1285, 1233 cm⁻¹; MS 337 [(M+1)—fumaric acid].

### EXAMPLE 2

### N-Phenyl-N-[1-(2-phenylethyl-1,1,2,2-d₄)piperidin-4-yl]propionamide-2,2,3,3,3-d₅ citrate

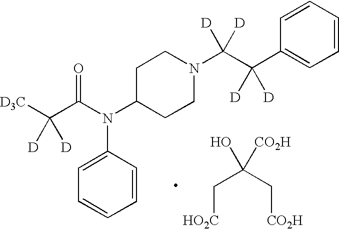

### Step 1

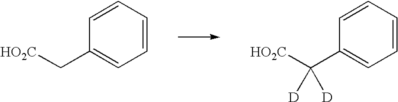

Phenylacetic acid-d₂: A mixture of phenylacetic acid (5.00 g; 36.72 mmol), anhydrous potassium carbonate (20.30 g; 146.88 mmol) and deuterium oxide (30 mL) was heated at about 100° C. for about 24 hours. The reaction mixture was cooled to about 0° C., acidified to pH 2 with 6N hydrochloric acid, and then extracted with dichloromethane. The organic layer was dried over sodium sulfate and concentrated in vacuo. This process was repeated two more times to give the title compound as a white solid (4.60 g; 91%). ¹H NMR (400 MHz, CDCl₃) δ 7.21-7.36 (m, 5H); IR (film) υ 3061, 1700, 1497, 1448, 1408, 1294 cm⁻¹; MS 137 (M−1).

### Step 2

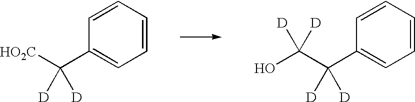

2-Phenylethanol-1,1,2,2-d₄: At about 0° C., a solution of phenylacetic acid-d₂ (4.00 g; 28.95 mmol) in tetrahydrofuran (30 mL) was added portionwise to a suspension of lithium aluminum deuteride (1.83 g; 43.59 mmol) in tetrahydrofuran (20 mL). The mixture was stirred at ambient temperature for 2-3 hours, and then cooled to about 0° C. After cold deuterium oxide (5 mL) was added, the mixture was filtered and the inorganic salts washed with ethyl acetate. The filtrate and washings were combined and concentrated in vacuo to give the title compound as a colorless liquid (3.65 g). ¹H NMR (400 MHz, CDCl₃) δ 7.18-7.34 (m, 5H); IR (film) υ 3363, 3067, 3027, 2219, 2090, 1597, 1495, 1444, 1385 cm⁻¹.

### Step 3

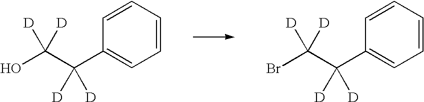

(2-Bromoethyl-1,1,2,2,-d₄)benzene: The procedure of Example 1, Step 2 was followed but substituting 2-phenylethanol-1,1,2,2-d₄ for 2-phenylethanol. The title compound was isolated as a liquid (5.00 g; 83%). ¹H NMR (400 MHz, CDCl₃) δ 7.16-7.36 (m, 5H); IR (film) υ 3060, 3026, 2925, 2853, 1495, 1448 cm⁻¹.

### Step 4

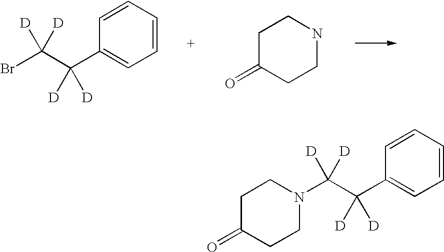

1-(2-Phenylethyl-1,1,2,2-d₄))piperidin-4-one: The procedure of Example 1, Step 3 was followed but substituting (2-bromoethyl-1,1,2,2,-d₄)benzene for (2-bromoethyl)benzene. The title compound was isolated as an oil, which solidified on standing (2.80 g; 5 1%). ¹H NMR (400 MHz, CDCl₃) δ 2.49 (t, J=6.1 Hz, 4H), 2.83 (t, J=6.1 Hz, 4H), 7.17-7.33 (m, 5H); IR (film) υ 2960, 2913, 2797, 1715 cm⁻¹; MS 208 (M+1).

### Step 5

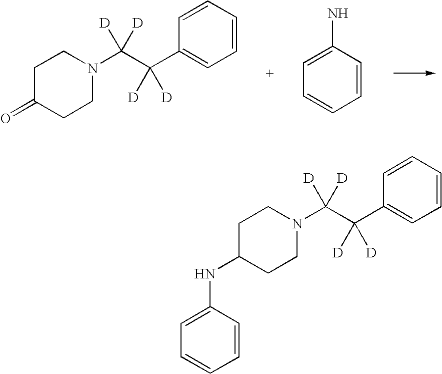

N-Phenyl-1-(2-phenylethyl-1,1,2,2-d₄)piperidin-4-amine: The procedure of Example 1, Step 4 was followed but substituting 1-(2-phenylethyl-1,1,2,2-d₄)piperidin-4-one for 1-(2-phenylethyl)piperidin-4-one. The title compound was isolated as a white solid (0.280 g; 41%). ¹H NMR (400 MHz, CDCl₃) δ 1.43-1.55 (m, 2H), 2.03-2.12 (m, 2H), 2.16-2.25 (m, 2H), 2.90-2.99 (m, 2H), 3.25-3.36 (m, 1H), 3.52 (br, exchangeable with D₂O, 1H), 6.61 (d, J=8.4 Hz, 2H), 6.68 (t, J=7.2 Hz, 1H), 7.12-7.31 (m, 7H); IR (KBr) υ 3274, 3026, 2928, 2788, 1599, 1494, 1317, 1266 cm⁻¹; MS 285 (M+1).

### Step 6

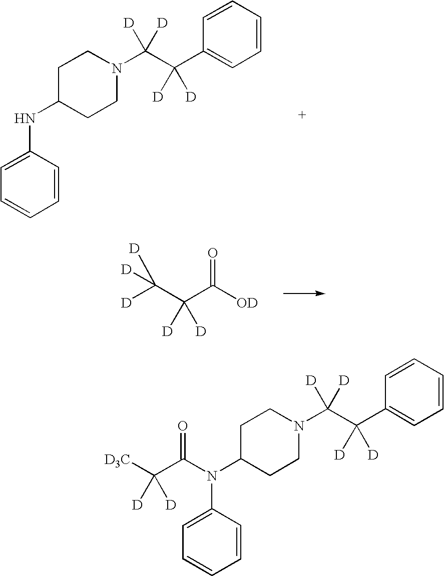

N-Phenyl-N-[1-(2-phenylethyl-1,1,2,2-d₄)piperidin-4-yl]propionamide-2,2,3,3,3-d₅: Thionyl chloride (0.05 mL, 0.685 mmol) was added to a solution of propionic acid-d₆ (0.05 mL; 0.669 mmol) in chloroform (1 mL). The mixture was stirred at ambient temperature for about 16 hours, and then cooled to about 0° C. A solution of N-phenyl-1-(2-phenylethyl-1,1,2,2-d₄)piperidin-4-amine (0.100 g; 0.352 mmol) in chloroform (1 mL) and triethylamine (0.14 mL; 1.004 mmol) were sequentially added dropwise to the mixture. The mixture was stirred at ambient temperature for about 30 minutes. Following standard extractive work up, the crude residue was purified by column chromatography on neutral alumina (10-20% ethyl acetate in petroleum ether) to yield the title compound as an oil, which solidified on standing (0.085 g; 70%). ¹H NMR (400 MHz, CDCl₃) δ 1.36-1.49 (m, 2H), 1.76-1.85 (m, 2H), 2.11-2.21 (m, 2H), 2.94-3.03 (m, 2H), 4.63-4.74 (m, 1H), 7.05-7.43 (m, 10H); IR (KBr) υ 2936, 2797, 2226, 2029, 1647, 1592, 1494, 1448, 1396, 1365, 1323, 1288, 1249 cm⁻¹; MS 346 (M+1).

### Step 7

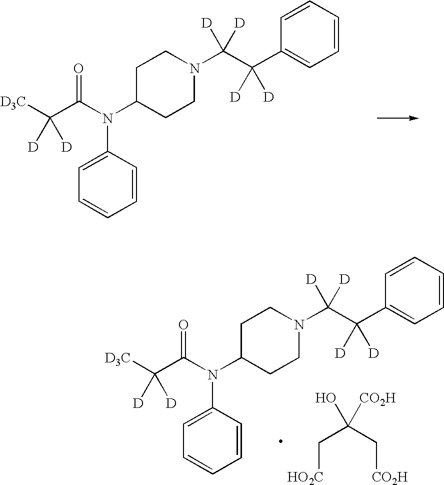

N-Phenyl-N-[1-(2-phenylethyl-1,1,2,2-d₄)piperidin-4-yl]propionamide-2,2,3,3,3-d₅ citrate: The procedure of Example 1, Step 6 was followed but substituting N-Phenyl-N-[1-(2-phenylethyl-1,1,2,2-d₄)piperidin-4-yl]propionamide-2,2,3,3,3-d₅ for N-Phenyl-N-[1-(2-phenylethyl)piperidin-4-yl]propionamide. The title compound was isolated as a white solid (0.095 g; 47%). m.p. 152-155° C.; ¹H NMR (400 MHz, pyridine-d₅) δ 1.37-1.57 (m, 2H), 1.69-1.84 (m, 2H), 1.98-2.14 (m, 2H), 2.83-3.02 (m, 2H), 3.52-3.71 (m, 4H), 4.78-4.91 (m, 1H), 7.09-7.45 (m, 10H), 12.08 (br, exchangeable with D₂O, 3H); IR (KBr) υ 3422, 3047, 2945, 1719, 1582, 1495, 1419, 1232 cm⁻¹; MS 346 [(M+1)—fumaric acid].

### EXAMPLE 3

### N-Phenyl-N-[1-(2-phenylethyl-1,1,2,2-d₄)piperidin-3,3-d₂-4-yl]propionamide-2,2,3,3,3-d₅ citrate

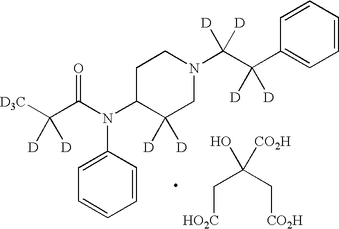

### Step 1

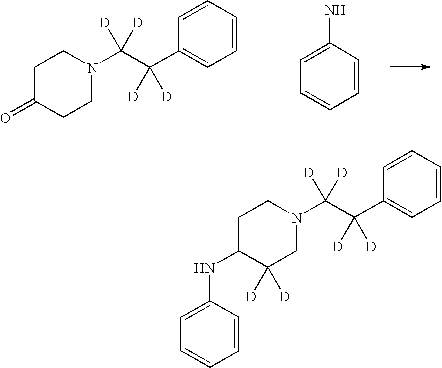

N-Phenyl-1-(2-phenylethyl-1,1,2,2-d₄)piperidin-3,3-d₂-4-amine: The pro Example 1, Step 4 was followed but substituting (2-bromoethyl-1,1,2,2,-d₄)benzene, for (2-bromoethyl)benzene and acetic acid-OD for acetic acid. The title compound was isolated as a white solid (0.710 g; 72%). ¹H NMR (400 MHz, CDCl₃) δ 1.42-1.54 (m, 1H), 2.02-2.13 (m, 1H), 2.14-2.26 (m, 2H), 2.89-3.00 (m, 2H), 3.26-3.36 (m, 1H), 3.52 (br, 1H), 6.60 (d, J=7.8 Hz, 2H), 6.68 (t, J=7.2 Hz, 1H), 7.12-7.32 (m, 7H); IR (KBr) υ 3276, 3026, 2922, 2786, 1599, 1526, 1493, 1318, 1269 cm⁻¹; MS 287 (M+1).

### Step 2

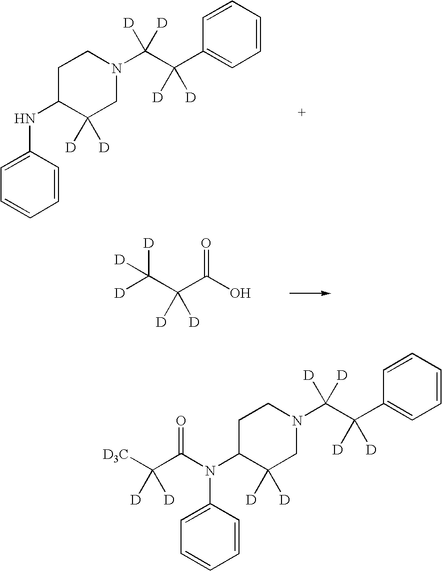

N-Phenyl-N-[1-(2-phenylethyl-1,1,2,2-d₄)piperidin-3,3-d₂-4-yl]propionamide-2,2,3,3,3-d₅: The procedure of Example 2, Step 6 was followed but substituting N-phenyl-1-(2-phenylethyl-1,1,2,2-d₄)piperidin-3,3-d₂-4-amine for N-phenyl-1-(2-phenylethyl-1,1,2,2-d₄)piperidin-4-amine. The title compound was isolated as an oil which solidified on standing (0.045 g; 66%). ¹H NMR (400 MHz, CDCl₃) δ 1.35-1.50 (m, 1H), 1.74-1.85 (m, 1H), 2.10-2.22 (m, 2H), 2.93-3.04 (m, 2H), 4.62-4.72 (m, 1H), 7.06-7.44 (m, 10H); IR (KBr) υ 2932, 2802, 1650, 1593, 1495, 1452, 1397, 1263 cm⁻¹; MS 348 (M+1).

### Step 3

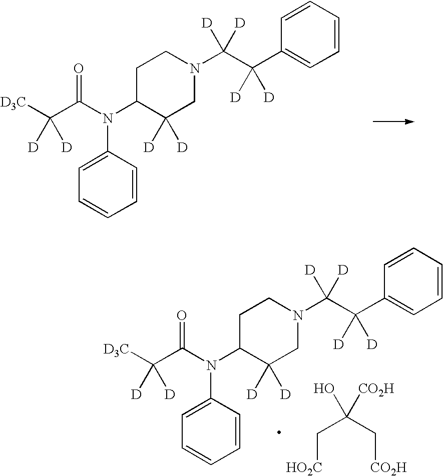

N-Phenyl-N-[1-(2-phenylethyl-1,1,2,2-d₄)piperidin-3,3-d₂-4-yl]propionamide-2,2,3,3,3-d₅ citrate: The procedure of Example 1, Step 6 was followed but substituting N-phenyl-N-[1-(2-phenylethyl-1,1,2,2-d₄)piperidin-3,3-d₂-4-yl]propionamide-2,2,3,3,3-d₅ for N-phenyl-N-[1-(2-phenylethyl)piperidin-4-yl]propionamide. The title compound was isolated as a white solid (0.075 g; 48%). m.p. 152-155° C.; ¹H NMR (400 MHz, pyridine-d₅) δ 1.38-1.53 (m, 1H), 1.72-1.82 (m, 1H), 1.97-2.11 (m, 2H), 2.86-2.97 (m, 2H), 3.61 (d, J=15.3 Hz, 2H), 3.70 (d, J=15.3 Hz, 2H), 4.79-4.88 (m, 1H), 7.10-7.45 (m, 10H), 11.31 (br, exchangeable with D₂O, 3H); IR (KBr) υ 3422, 3046, 2944, 1719, 1583, 1495, 1418, 1360, 1289, 1232 cm⁻¹; MS 348 [(M+1)—fumaric acid].

### EXAMPLE 4

### N-(4-(methoxymethyl)-1-(2-(thiophen-2-yl)ethyl)piperidin-4-yl)-N-phenylpropionamide citrate

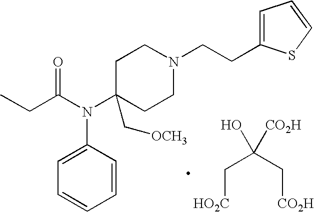

### Step 1

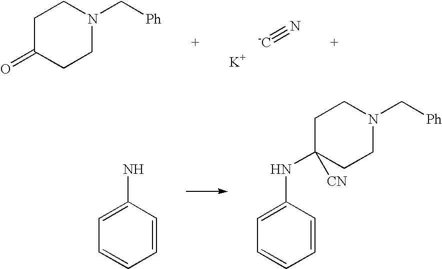

1-Benzyl-4-(phenylamino)piperidine-4-carbonitrile: While maintaining the internal temperature of the mixture at 5-8° C., glacial acetic acid (250 g; 238 mL, 5.0 mol) was added dropwise to a stirred mixture of 1-benzyl-4-piperidone (30.0 g; 0.159 mol), aniline (59.0 g; 0.635 mol), potassium cyanide (41.3 g; 0.635 mol) and dichloromethane (316 mL) over a period of two hours. After the addition was complete, the reaction mixture was gradually warmed to about 50° C. over a period of about 1 hour. The mixture was stirred at this temperature for about 17 hours. It was then cooled, and crushed ice (2 kg) was added. While maintaining the internal temperature of the mixture below 20° C., a 10% aqueous sodium hydroxide solution (180 g) was added dropwise over a period of 1 hour. The mixture was then extracted with dichloromethane. The aqueous layer was treated with an aqueous permanganate solution to destroy excess potassium cyanide, before discarding. The organic layer was washed with water and brine, dried over sodium sulfate and concentrated to obtain a brown colour residue, which was triturated with 2-propanol to obtain a solid. This was solid was collected by filtration, washed with cold 2-propanol and dried to give the title compound as an off-white solid (37.0 g; 80%). m.p.142-146° C.; ¹H NMR (400 MHz, CDCl₃) δ 1.98-2.01 (m, 1H), 2.35 (d, J=13.2 Hz, 2H), 2.53 (t, J=11.0 Hz, 2H), 2.86-2.88 (m, 2H), 3.62 (s, 2H), 3.67 (br, exchangeable with D₂O, 1H), 6.90-6.93 (m, 3H), 7.22-7.29 (m, 7H), IR (KBr) υ 3404, 3316, 3254, 3102, 3030, 2947, 2808, 2757, 2663, 2497, 2442, 2227, 1920, 1599, 1498, 1448, 1312, 1259, 1166, 1100, 749, 696 cm⁻¹; MS 292 (M+1).

### Step 2

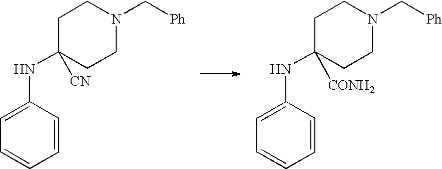

1-Benzyl-4-(phenylamino)piperidine-4-carboxamide: At about 0° C., 1-benzyl-4-(phenylamino)piperidine-4-carbonitrile (24.0 g; 82.47 mmol) was added portion wise to 95% sulfuric acid (311 g; 3.01 mol) over a period of about 1 hour. The mixture was stirred at ambient temperature for about 16 hours. The mixture was poured onto crushed ice, and then stirred for about 1 hour. The resulting precipitate was collected by filtration to form a wet cake. The wet cake was dissolved in water (300 mL), cooled to about 0° C., and basified to pH greater than 12 using a 10% aqueous sodium hydroxide solution. The resulting precipitate was washed with water and dried to give the title compound as a white solid (20.0 g; 78%). m.p. 187-190° C.; ¹H NMR (400 MHz, DMSO-d₆) δ 1.81-1.84 (m, 2H), 2.01 (t, J=10.4 Hz, 2H), 2.21 (t, J=10.8 Hz, 2H), 2.50-2.53 (m, 2H), 3.35 (s, 2H), 5.48 (br s, exchangeable proton with D₂O, 1H), 6.54-6.59 (m, 3H), 7.00-7.07 (m, 3H), 7.21 (br, exchangeable proton with D₂O, 1H), 7.28-7.32 (m, 4H). IR (KBr) υ 3439, 3351, 3060, 3020, 2957, 2807, 2769, 1676, 1602, 1498, 1373, 1165, 740, 695 cm⁻¹; MS 310 (M+1).

### Step 3

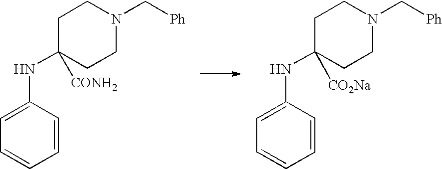

Sodium 1-benzyl-4-(phenylamino)piperidine-4-carboxylate: A mixture of 1-benzyl-4-(phenylamino)piperidine-4-carboxamide (20.0 g; 64.7 mmol), potassium hydroxide (14.35 g; 256 mmol) and ethane-1,2-diol (120 mL) was heated at reflux for about 24 hours. The mixture was diluted with distilled water (200 mL), cooled to about 0° C., and acidified to a pH of about 2.0 with with conc. hydrochloric acid. At 0-5° C., the mixture was then basified to pH greater than 12 with a 30% aqueous sodium hydroxide solution, and then stirred at 0-5° C. for about 1 hour. The resulting precipitate was collected by filtration and dried in vacuo to form a slightly wet cake. At about 60° C., this slightly wet cake was dissolved in distilled water (50 mL), and then 2-propanol (50 mL) was added. After cooling to ambient temperature, the resulting precipitate was collected by filtration, washed successively with a mixture of 2-propanol-water (4: 1) and 2-propanol, and then dried in vacuo to give the title compound as an off-white solid (12.0 g; 60%). m.p. 323-327° C.; ¹H NMR (400 MHz, DMSO-d₆) δ 1.72-7.75 (m, 2H), 1.96 (t, J=10 Hz, 2H), 2.24-2.32 (m, 2H), 2.42-2.45 (m, 2H), 3.39 (s, 2H), 5.05 (br, exchangeable with D₂O, 1H), 6.34 (t, J=7.0 Hz, 1H), 6.60 (d, J=8 Hz, 2H), 6.87-6.89 (m, 2H), 7.16-7.31 (m, 5H). IR (KBr) υ 3575, 3419, 3382, 3064, 2950, 2813, 2760, 1595, 1499, 1379, 1319, 1164, 1067, 748, 697 cm⁻¹ MS 311 (M+1).

### Step 4

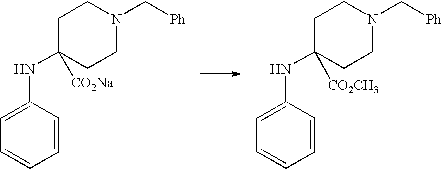

Methyl 1-benzyl-4-(phenylamino)piperidine-4-carboxylate: At 70-72° C., iodomethane (5.49 g; 2.41 mL; 38.7 mmol) was added dropwise to a suspension of sodium 1-benzyl-4-(phenylamino)piperidine-4-carboxylate (12.0 g; 38.7 mmol) in dry dimethyl sulfoxide (120 mL). After the addition was complete, the clear pale yellow solution was stirred for about 10 minutes, and then poured into ice-cold water. Following standard extractive workup with n-hexane, the title compound was isolated as a white crystalline solid (5.30 g; 42.2%). m.p. 76-79° C.; ¹H NMR (400 MHz, CDCl₃) δ 2.02 (d, J=13.6 Hz, 2H), 2.20-2.27 (m, 2H), 2.41 (t, J=10 Hz, 2H), 2.57-2.60 (m, 2H), 3.50 (s, 2H), 3.67 (s, 3H), 3.84 (br, exchangeable with D₂O, 1H), 6.56 (d, J=8 Hz, 2H), 6.74 (t, J=7.4 Hz, 1H), 7.13 (t, J=7.8 Hz, 2H), 7.25-7.31 (m, 5H). IR (film) υ 3401, 3031, 2945, 2813, 2771, 1728, 1600, 1502, 1443, 1318, 1256, 1147, 1070, 1020, 745, 695 cm⁻¹ MS 325 (M+1).

### Step 5

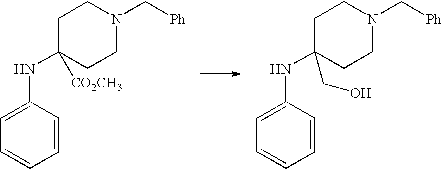

(1-Benzyl-4-(phenylamino)piperidin-4-yl)methanol: At about 0° C., a solution of methyl 1-benzyl-4-(phenylamino)piperidine-4-carboxylate (5.00 g; 15.43 mmol) in dichloromethane (10 mL) was added dropwise to a suspension of lithium aluminum hydride (1.17 g; 30.86 mmol) in dry tetrahydrofuran (100 mL). The mixture was stirred at ambient temperature for about 16 hours. The mixture was cooled to about 0° C. and ice-cold water (1 mL) was added. The mixture was filtered and the inorganic salts washed with ethyl acetate. The filtrate and washings were combined and concentrated in vacuo to give the title compound as a white solid (4.10 g; 90%). m.p. 71-75° C.; ¹H NMR (400 MHz, CDCl₃) δ 1.67-1.70 (m, 2H), 1.85-1.93 (m, 2H), 2.28 (t, J=10 Hz, 2H), 2.37 (br exchangeable proton with D₂O, 1H), 2.56-2.61 (m, 2H), 3.49 (s, 2H), 3.63 (s, 2H), 6.78 (d, J=7.6 Hz, 2H), 6.84 (t, J=7.4 Hz, 1H), 7.18 (t, J=7.8 Hz, 2H), 7.25-7.32 (m, 5H). IR (film) υ 3401, 3032, 2936, 2815, 2765, 2672, 1599, 1497, 1451, 1320, 1051, 744, 695 cm⁻¹ MS 297 (M+1).

### Step 6

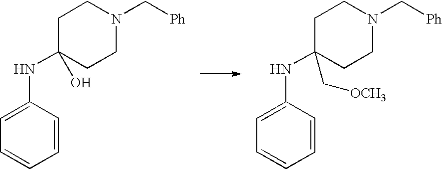

1-Benzyl-4-(methoxymethyl)-N-phenylpiperidin-4-amine: A solution of (1-benzyl-4-(phenylamino)piperidin-4-yl)methanol (1.0 g, 3.37 mmol) in tetrahydrofuran (15 mL) was added dropwise to a stirred suspension of sodium hydride (60% in mineral oil; 0.162 g; 4.05 mmol) in dry tetrahydrofuran (25 mL). The mixture was stirred at ambient temperature for about 30 minutes, and then a solution of iodomethane (270 μL; 4.34 mmol) in tetrahydrofuran (10 mL) was added dropwise. The mixture was stirred at ambient temperature for about 2 hours, cooled to about 0° C., and then water (1 mL) was added to destroy excess sodium hydride. Standard extractive work up provided a crude residue which was purified by silica gel column chromatography (2% methanol in chloroform) to give the title compound as an oil (0.700 g; 67%). ¹H NMR (400 MHz, CDCl₃) δ 1.70-1.77 (m, 2H), 1.90-1.93 (m, 2H), 2.46-2.50 (m, 2H), 2.55-2.58 (m, 2H), 3.30 (s, 3H), 3.33 (s, 2H), 3.52 (s, 2H), 6.79-6.82 (m, 3H), 7.15 (t, J=7.8 Hz, 2H), 7.25-7.31 (m, 5H). IR (film) υ 3408, 3338, 3028, 2928, 2878, 2813, 2769, 1599, 1497, 1451, 1320, 1253, 1185, 1100, 743, 696 cm⁻¹ MS 311 (M+1).

### Step 7

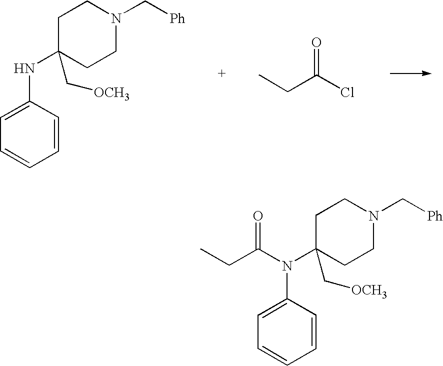

N-(1-Benzyl-4-(methoxymethyl)piperidin-4-yl)-N-phenylpropionamide: At about 0° C., propionyl chloride (270 mg; 2.92 mmol) was added to a mixture of 1-benzyl-4-(methoxymethyl)-N-phenylpiperidin-4-amine (700 mg; 2.25 mmol), triethylamine (30 μL; catalytic) and dichloromethane (10 mL). The reaction mixture was stirred at ambient temperature for about 3 hours. Standard extractive work up gave the title compound as pale yellow oil (0.400 g; 48%). ¹H NMR (400 MHz, CDCl₃) δ 0.92 (t, J=7.2 Hz, 3H), 1.66-1.72 (m, 2H), 1.81 (q, J=7.3 Hz, 2H), 2.11-2.20 (m, 4H), 2.55-2.58 (m, 2H), 3.41 (s, 3H), 3.45 (s, 2H), 4.05 (s, 2H), 7.20-7.34 (m, 10H), IR (film) υ 3062, 3030, 2978, 2931, 2812, 2765, 1728, 1654, 1592, 1539, 1488, 1456, 1367, 1249, 1108, 1023, 747, 702, cm⁻¹ MS 367 (M+1).

### Step 8

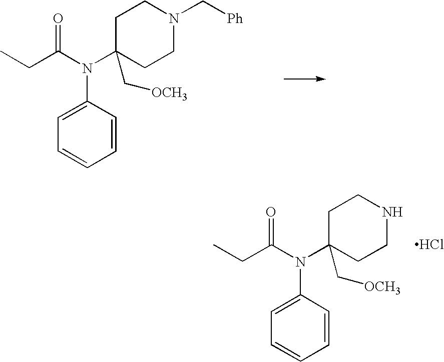

N-(4-(Methoxymethyl)piperidin-4-yl)-N-phenylpropionamide hydrochloride: At about 50° C., a mixture of N-(1-benzyl-4-(methoxymethyl)piperidin-4-yl)-N-phenylpropionamide (0.250 g; 0.682 mmol), 20% palladium hydroxide on carbon (0.030 g), and ethanol (20 mL) was hydrogenated at 60 psi for about 2 hours. After filtering with a pad of Celite, the filtrate was concentrated in vacuo to yield a pale yellow liquid. Diethyl ether (5.0 mL) was added to the pale yellow liquid, and then a saturated solution of hydrochloric gas in diethyl ether (1.0 mL) was added. After stirring for about 30 minutes, the solution was filtered and dried to obtain the title compound as off-white solid (0.110 g; 52%). ¹H NMR (400 MHz, CD₃OD) δ 0.91 (t, J=7.4 Hz, 3H), 1.88 (q, J=7.4 Hz, 2H), 2.03-2.09 (m, 2H), 2.29-2.34 (m, 2H), 3.10-3.15 (m, 4H), 3.43 (s, 3H), 4.03 (s, 2H), 7.39-7.46 (m, 5H). IR (film) υ 3423, 3057, 2978, 2935, 2813, 2729, 1652, 1593, 1486, 1456, 1372, 1305, 1249, 1190, 1107, 987 cm⁻¹ MS 277 (M+1)

### Step 9

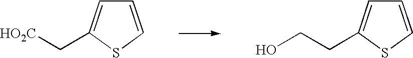

2-(Thiophen-2-yl)ethanol: At about 0° C., a solution of thiophen-2-yl-acetic acid (1.0 g; 7.03 mmol) in tetrahydrofuran (10 mL) was added dropwise to a suspension of lithium aluminum hydride (0.534 g; 14.05 mmol) in dry tetrahydrofuran (10 mL). The mixture was stirred at ambient temperature for about 4 hours, and then cooled to about 0° C. After adding a cold saturated sodium chloride solution (1 mL), the mixture was filtered, and the inorganic salts were washed with tetrahydrofuran and ethyl acetate. The filtrate and washings were combined and concentrated in vacuo to give the title compound as brown oil (0.600 g; 66.5%). ¹H NMR (400 MHz, CDCl₃) δ 1.60 (br, exchangeable with D₂O, 1H), 3.08 (t, J=6.2 Hz, 2H), 3.85 (t, J=6.2 Hz, 2H), 6.87-6.88 (m, 1H), 6.95-6.97 (m, 1H), 7.16-7.25 (m, 1H). IR (film) υ 3345, 3105, 2211, 2126, 2090, 1792, 1433, 1138, 972, 737, 699 cm⁻¹ MS: 129 (M+1).

### Step 10

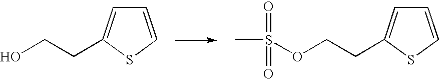

2-(Thiophen-2-yl)ethyl methanesulfonate: At about 0° C., methanesulfonyl chloride (200 μL; 2.58 mmol) was added dropwise to a mixture of 2-(thiophen-2-yl)ethanol (0.250 g; 1.95 mmol), triethylamine (0.40 mL; 2.87 mmol) and dichloromethane (5 mL). The mixture was stirred at about 0° C. for about 3 hours. Standard extractive work up provided the title compound as yellow oil which was used in the next step without further purification (0.3 10 g; 77%). ¹H NMR (400 MHz, CDCl₃) δ 2.92 (s, 3H), 3.27 (t, J=6.6 Hz, 2H), 4.42 (t, J=6.6 Hz, 2H), 6.90-6.91 (m, 1H), 6.95-6.97 (m, 1H), 7.19 (d, J=4.8 Hz, 1H). IR (KBr) υ 3108, 3060, 2946, 1462, 1425, 1351, 1173, 967, 736, 706 cm⁻¹.

### Step 11

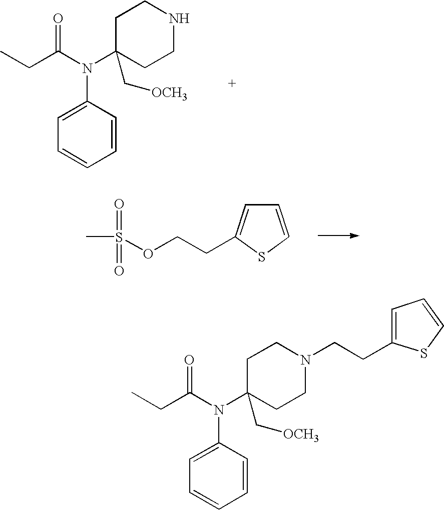

N-(4-(Methoxymethyl)-1-(2-(thiophen-2-yl)ethyl)piperidin-4-yl)-N-phenylpropionamide: A mixture of N-(4-(methoxymethyl)piperidin-4-yl)-N-phenylpropionamide (0.190 g; 0.687 mmol), 2-(thiophen-2-yl)ethyl methanesulfonate (0.170 g; 0.825 mmol), triethylamine (190 μL; 1.38 mmol), anhydrous potassium carbonate (0.029 g; 0.210 mmol), potassium iodide (0.005 g; catalytic) and acetonitrile (5 ML) was heated at reflux for about 6 hours. The mixture was filtered, and the filtrate was concentrated in vacuo to provide a crude residue which was purified by preparative HPLC on a Kromasil 100 C18 (250×30 mm, 5 μ column, eluting with acetonitrile/0.05% formic acid (gradient) at a flow rate of 42 mL/min). The title product eluted at 5.5 minutes. Acetonitrile was removed by distillation and the remaining aqueous phase was basified to a pH of about 9.0 with a 10% sodium carbonate solution. Standard extractive workup with ethyl acetate gave the title compound as a yellow solid (0.090 g; 34%). The obtained free base was used for the next step without any further purification.

### Step 12

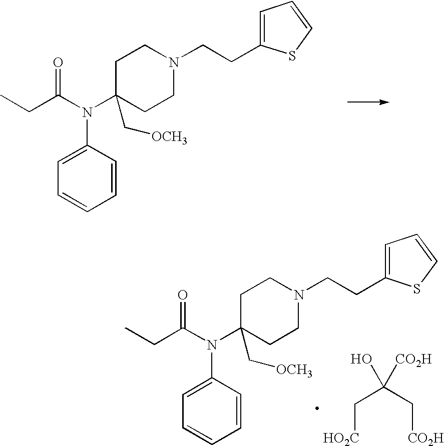

N-(4-(methoxymethyl)-1-(2-(thiophen-2-yl)ethyl)piperidin-4-yl)-N-phenylpropionamide citrate: A solution of N-(4-(methoxymethyl)-1-(2-(thiophen-2-yl)ethyl)piperidin-4-yl)-N-phenylpropionamide (30 mg; 0.078 mmol) in methanol (1.5 mL) was added dropwise to a solution of citric acid monohydrate (15 mg; 0.071 mmol) in methanol (1.5 mL). The mixture was stirred at ambient temperature for about 1 hour. The solvent was removed in vacuo and the resulting residue was triturated with n-pentane to yield the title compound as an off-white solid (25 mg; 54%). m.p. Foamy mass 57° C. and completed 105° C., NMR (400 MHz, pyridine-d₅) δ 1.01 (t, J=7.4 Hz, 3H), 1.88-1.93 (m, 4H), 2.21-2.32 (m, 2H), 2.43-2.46 (m, 2H), 2.62-2.67 (m, 4H), 3.01-3.02 (m, 2H), 3.37 (s, 3H), 3.64-3.77 (m, 4H), 4.25 (s, 2H), 6.92-7.00 (m, 2H), 7.26-7.28 (m, 1H), 7.35-7.45 (m, 6H), IR (KBr) υ 3435, 2982, 2930, 2821, 2576, 1832, 1731, 1643, 1592, 1486, 1451, 1379, 1243, 1190, 1105, 1019, 969 cm⁻¹: MS 387 (M+1).

### EXAMPLE 5

### N-(4-(methoxymethyl)-1-(2-(thiophen-2-yl-1,1,2,2-d₄)ethyl)piperidin-4-yl)-N-phenylpropionamide citrate

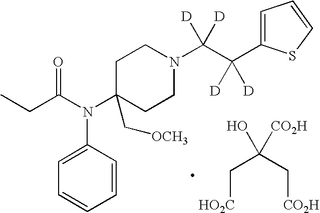

### Step 1

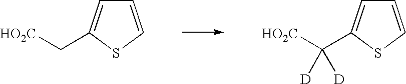

2,2-d₂-2-(thiophen-2-yl)acetic acid: A mixture of thiophen-2-yl-acetic acid (3.00 g; 21.12 mmol), anhydrous potassium carbonate (11.60 g; 84.5 mmol) and deuterium oxide (12 mL) was heated at about 100° C. for about 18 hours. After distillation, fresh deuterium oxide (12 mL) was added and heating continued for about 16 hours. This cycle was repeated once more. The reaction mixture was cooled to about 0° C., and acidified to a pH of about 3.0 by adding 20%-d₁-hydrochloric acid in deuterium oxide. Standard extractive workup with dichloromethane gave the title compound as an off-white solid (2.70 g; 89%). m.p. 64-68° C.; ¹H NMR (400 MHz, D₂O) δ 6.93-6.94 (m, 1H), 7.01-7.03 (m, 1H), 7.31-7.32 (m, 1H), IR (KBr) υ 3166, 3100, 3031, 2916, 2132, 1702, 1409, 1304, 1228, 1188, 1035, 937, 895, 694 cm⁻¹ MS 143 (M−1)

### Step 2

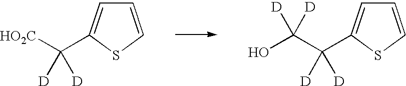

1,1,2,2-d₄-2-(thiophen-2-yl)ethanol: At about 0° C., a solution of 2,2-d₂-2-thiophen-2-yl)acetic acid (2.70 g; 18.75 mmol) in dry tetrahydrofuran (20 mL) was added dropwise to a suspension of lithium aluminum deuteride (1.73 g; 41.20 mmol) in dry tetrahydrofuran (30 mL). The mixture was stirred at ambient temperature for about 3 hours, cooled to about 0° C., and deuterium oxide (5 mL) was added. The mixture was filtered and the inorganic salts washed with dry dichloromethane. The filtrate and washings were combined and concentrated in vacuo to give the title compound as a brown oil (2.00 g; 81%). ¹H NMR (400 MHz, CDCl₃) δ 6.87-6.88 (m, 1H), 6.95-6.97 (m, 1H), 7.17-7.18 (m, 1H), IR (film) υ 3363, 3101, 3071, 2211, 2089, 1634, 1433, 1232, 1199, 1138, 972, 901, 698 cm⁻¹ MS 133 (M+1)

### Step 3

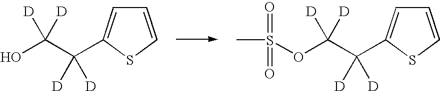

1,1,2,2-d₄-2-(thiophen-2-yl)ethyl methanesulfonate: At about 0° C., methanesulfonyl chloride (1.12 g; 9.78 mmol) was added dropwise to a mixture of 1,1,2,2-d₄-2-(thiophen-2-yl)ethanol (1.00 g; 7.57 mmol), triethylamine (1.15 g; 11.36 mmol) and dichloromethane (10 mL). The mixture was stirred at about 0° C. for about 2 hours, and then concentrated in vacuo to obtain a crude residue. The crude residue was then purified by silica gel column chromatography (5% ethyl acetate in petroleum ether) to give the title compound as a pale yellow oil (1.20 g; 75%). ¹H NMR (400 MHz, CDCl₃) δ 2.93 (s, 3H), 6.90-6.91 (m, 1H), 6.95-6.97 (m, 1H), 7.18-7.20 (m, 1H); IR (film) υ 3107, 3078, 3025, 2935, 2855, 2522, 2252, 1349, 1176, 1145, 1084, 972, 859, 812, 701 cm⁻¹; MS 211 (M+1).

### Step 4

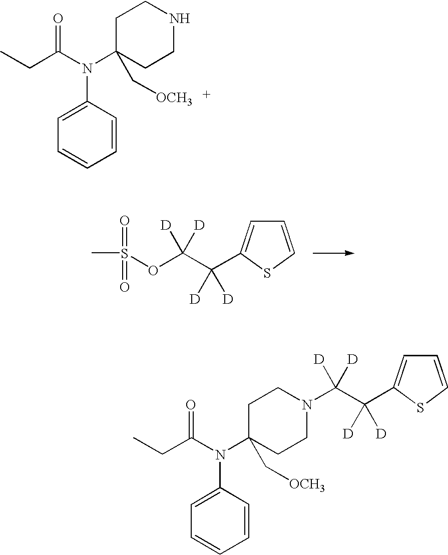

N-(4-(Methoxymethyl)-1-(1,1,2,2-d₄-2-(thiophen-2-yl)ethyl)piperidin-4-yl)-N-phenylpropionamide: The procedure of Example 4, Step 11 was followed but substituting, 1,1,2,2-d₄-2-(thiophen-2-yl)ethyl methanesulfonate for 2-(thiophen-2-yl)ethyl methanesulfonate. The title compound was isolated as a yellow oily mass (80 mg; 42%). The obtained free base was used for the next step immediately.

### Step 5

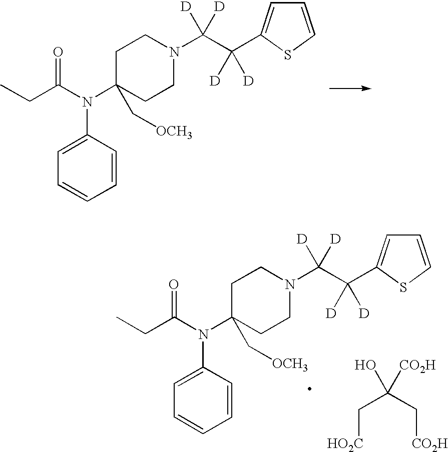

N-(4-(Methoxymethyl)-1-(1,1,2,2-d₄-2-(thiophen-2-yl)ethyl)piperidin-4-yl)-N-phenylpropionamide citrate: The procedure of Example 4, Step 12 was followed, but substituting N-(4-(Methoxymethyl)-1-(1,1,2,2-d₄-2-(thiophen-2-yl)ethyl)piperidin-4-yl)-N-phenylpropionamide for N-(4-(Methoxymethyl)-1-(2-(thiophen-2-yl)ethyl)piperidin-4-yl)-N-phenylpropionamide. The title compound was isolated as an off-white solid (145 mg; 86%). m.p. Foamy mass 50° C. and completed 120° C., ¹H NMR (400 MHz, pyridine-d₅) δ 1.02(t, J=7.4 Hz, 3H), 1.90-1.93 (m, 4H), 2.18-2.28 (m, 2H), 2.38-2.48 (m, 2H), 2.60-2.68 (m, 2H), 3.37 (s, 3H), 3.61-3.75 (m, 4H), 4.24 (s, 2H), 6.90-6.92 (m, 1H), 6.96-7.00 (m, 1H), 7.25-7.27 (m, 1H), 7.39-7.43 (m, 5H), IR (KBr) υ 3440, 2978, 2929, 2819, 2708, 2567, 2230, 1727, 1643, 1591, 1490, 1452, 1377, 1313, 1246, 1193, 1107, 1018, 939 cm⁻¹; MS 391 [(M+1)

### EXAMPLE 6

### N-(4-(methoxy-d₃-methyl)-1-(2-(thiophen-2-yl-1,1,2,2-d₄)ethyl)piperidin-4-yl)-N-phenylpropionamide citrate

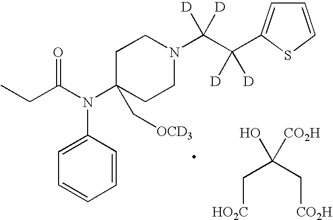

### Step 1

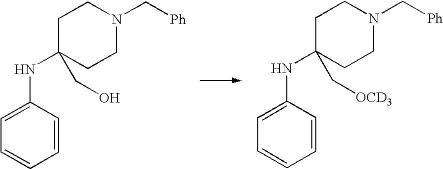

1-Benzyl-4-(d₃-methoxymethyl)-N-phenylpiperidin-4-amine: The procedure of Example 4, Step 6 was followed, but substituting d₃-iodomethane for iodomethane. The title compound was isolated as an oil (0.700 g; 67%). ¹H NMR (400 MHz, CDCl₃) δ 1.70-1.77 (m, 2H), 1.90-1.93 (m, 2H), 2.43-2.49 (m, 2H), 2.54-2.58 (m, 2H), 3.32 (s, 2H), 6.79-6.83 (m, 3H), 7.15 (t, J=7.6 Hz, 2H), 7.23-7.25 (m, 1H), 7.28-7.31 (m, 4H); IR (film) υ 3407, 3335, 3178, 3029, 2933, 2857, 2811, 2769, 2238, 2187, 2053, 1599, 1497, 1452, 1359, 1318, 1254, 1128, 992, 744 cm⁻¹; MS 314 (M+1).

### Step 2

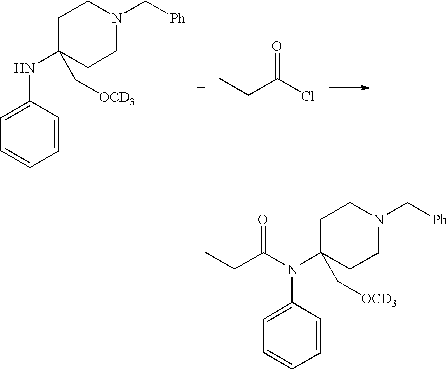

N-(1-Benzyl-4-(d₃-methoxymethyl)piperidin-4-yl)-N-phenylpropionamide: The procedure of Example 4, Step 7 was followed but substituting 1-benzyl-4-(d₃-methoxymethyl)-N-phenylpiperidin-4-amine for 1-benzyl-4-(methoxymethyl)-N-phenylpiperidin-4-amine. The title compound was isolated as a pale yellow oil (0.40 g; 48%). ¹H NMR (400 MHz, CDCl₃) δ 0.92 (t, J=7.4 Hz, 3H), 1.78-1.82 (m, 2H), 1.99-2.17 (m, 3H) 2.20-2.33 (m, 3H), 2.50-2.60 (m, 2H), 3.45-3.51 (m, 2H), 4.03 (s, 2H) 7.29-7.31 (m, 10H); IR (KBr) υ 3062, 2968, 2930, 2808, 2769, 2238, 2188, 1655, 1593, 1487, 1454, 1366, 1251, 1128, 1081, 1024, 741, 702 cm⁻¹; MS 370 (M+1).

### Step 3

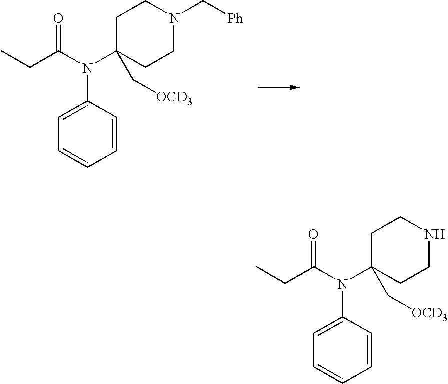

N-(4-(d₃-Methoxymethyl)piperidin-4-yl)-N-phenylpropionamide: The procedure of Example 4, Step 8 was followed but substituting N-(1-benzyl-4-(d₃-methoxymethyl)piperidin-4-yl)-N-phenylpropionamide for N-(1-benzyl-4-(methoxymethyl)piperidin-4-yl)-N-phenylpropionamide. The title compound was isolated as a pale yellow liquid (450 mg; 74.5%). The obtained free base was used as such in the next step with out further purification.

### Step 4

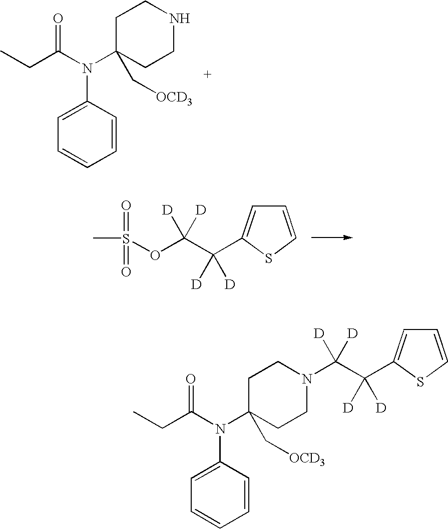

N-(4-(d₃-Methoxymethyl)-1-(1,1,2,2-d₄-2-(thiophen-2-yl)ethyl)piperidin-4-yl-N-phenylpropionamide: The procedure of Example 5, Step 4 was followed but substituting N-(4-(d₃-Methoxymethyl)piperidin-4-yl)-N-phenylpropionamide for N-(4-(Methoxymethyl)piperidin-4-yl)-N-phenylpropionamide. The title compound was isolated as a yellow oily mass (70 mg; 25%). The obtained free base was used for the next step without any further purification.

### Step 5

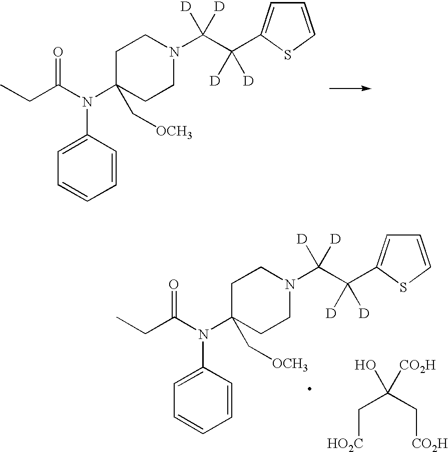

N-(4-(d₃-Methoxymethyl)-1-(1,1,2,2-d₄-2-(thiophen-2-yl)ethyl)piperidin-4-yl)-N-phenylpropionamide citrate: The procedure of Example 5, Step 5 was followed but substituting N-(4-(d₃-methoxymethyl)-1-(1,1,2,2-d₄-2-(thiophen-2-yl)ethyl)piperidin-4-yl)-N-phenylpropionamide for N-(4-(methoxymethyl)-1-(1,1,2,2-d₄-2-(thiophen-2-yl)ethyl)piperidin-4-yl)-N-phenylpropionamide. The title compound was isolated as an off-white solid (85 mg; 62%). m.p. 58-62° C., ¹H NMR (400 MHz, pyridine-d₅) δ 0.99 (t, J=8.0 Hz, 3H), 1.85-1.91 (m, 4H), 2.12-2.15 (m, 2H), 2.38-2.41 (m, 2H), 2.56-2.59 (m, 2H), 4.21 (s, 2H), 4.96 (br exchangeable proton with D₂O, 1H), 6.88-6.89 (m, 1H), 6.94-6.96 (m, 1H), 7.23-7.25 (m, 1H), 7.32-7.42 (m, 5H); IR (KBr) υ 3434, 2977, 2931, 2876, 2570, 2241, 2197, 2120, 2058, 1728, 1644, 1592, 1488, 1453, 1375, 1312, 1246, 1194, 1125, 1080, 1015, 987 cm⁻¹; MS 394 (M+1).

### EXAMPLE 7

### N-[1-(2-(4-ethyl-5-oxo-4,5-dihydro-1H-tetrazol-1-yl)ethyl)-4-(methoxymethyl)piperidin-4-yl]-N-phenylpropionamide hydrochloride

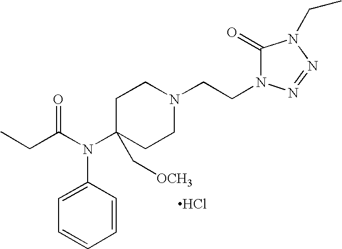

### Step 1

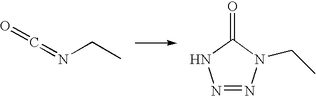

1-Ethyl-1H-tetrazol-5(4H)-one: A solution of anhydrous aluminum chloride (10.34 g; 77.55 mmol) in dry tetrahydrofuran (80 mL) was added all at once to a vigorously stirred mixture of ethyl isocyanate (5.00 g; 70.34 mmol), sodium azide (10.29 g; 158.28 mmol) and dry tetrahydrofuran (80 mL). The reaction mixture was stirred at reflux for about 24 hours, cooled to ambient temperature, and acidified to a pH of about 3 with 6N hydrochloric acid. The resulting biphasic mixture was concentrated to dryness in vacuo. Following standard extractive workup with hot acetone, the crude residue was purified by silica gel column chromatography (20% methanol in chloroform) to give the title product as a pale yellow solid (1.50 g; 19%). m.p. 77-80° C.; ¹H NMR (400 MHz, CDCl₃) δ 1.47 (t, J=7.4 Hz, 3H), 4.04 (q, J=7.3 Hz, 2H), 13.2 (br, exchangeable with D₂O, 1H): IR (film) υ 3403, 3245, 3148, 3059, 2999, 2866, 2766, 2686, 1750, 1697, 1455, 1397, 1342, 1198, 1052, 958, 804, 734, 666 cm⁻¹ MS 113 (M−1).

### Step 2

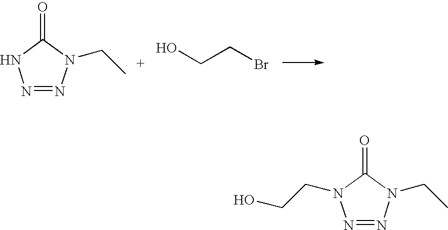

1-Ethyl-4-(2-hydroxyethyl)-1H-tetrazol-5(4H)-one: A mixture of 1-ethyl-1H-tetrazol-5(4H)-one (200 mg; 1.75 mmol), 2-bromoethanol (240 mg; 1.92 mmol), anhydrous potassium carbonate (361 mg; 2.62 mmol) and acetonitrile (10 mL) was heated at reflux for about 4 hours. The reaction mixture was filtered and the filtrate was concentrated in vacuo to provide a crude residue which was purified by silica gel column chromatography (10% methanol in chloroform) to give the title product as a pale yellow liquid (210 mg; 76%). ¹H NMR (400 MHz, CDCl₃) δ 1.45 (t, J=7.4 Hz, 3H), 3.01 (br, exchangeable with D₂O, 1H), 4.0-4.04 (m, 4H), 4.14-4.19 (m, 2H); IR (film) υ 3421, 2988, 1711, 1409, 1230, 974, 743 cm⁻¹ MS 159 (M+1).

### Step 3

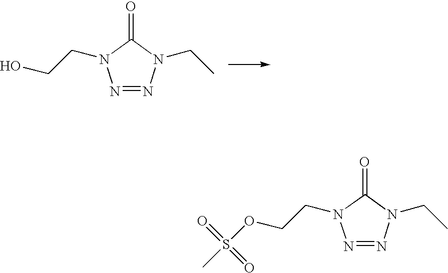

2-(4-Ethyl-5-oxo-4,5-dihydro-1H-tetrazol-1-yl)ethyl methanesulfonate: At about 0° C. methanesulfonyl chloride (198 mg, 1.73 mmol) was added dropwise to a mixture of 1-ethyl-4-(2-hydroxyethyl)-1H-tetrazol-5(4H)-one (250 mg; 1.58 mmol), triethylamine (331 μL; 2.36 mmol) and dichloromethane (20 mL). The reaction mixture was stirred at ambient temperature for about 1 hour. Standard extractive work up provided the title compound as a pale yellow oil which was used in the next step without further purification (250 mg; 84%). ¹H NMR (400 MHz, CDCl₃) δ 1.45 (t, J=7.4 Hz, 3H), 3.04 (s, 3H), 4.01 (q, J=7.2 Hz, 2H), 4.28-4.31 (m, 2H), 4.56-4.59 (m, 2H); IR (film) υ 3016, 2991, 1723, 1634, 1412, 1350, 1174, 1009, 906, 805, 737 cm⁻¹; MS 237 (M+1).

### Step 4

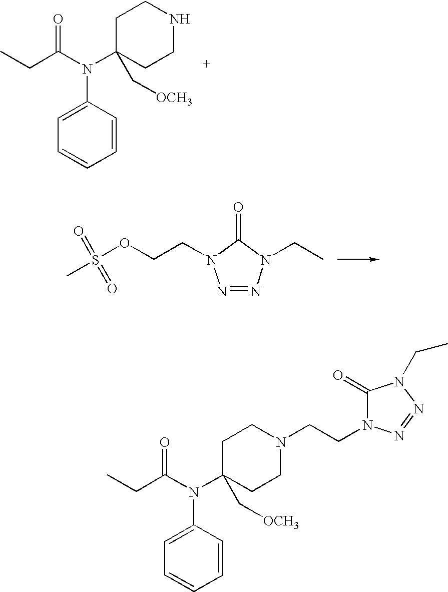

N-[1-(2-(4-Ethyl-5-oxo-4,5-dihydro-1H-tetrazol-1-yl)ethyl)-4-(methoxymethyl)piperidin-4-yl]-N-phenylpropionamide: A mixture of N-[4-(methoxymethyl)piperidin-4-yl]-N-phenylpropionamide (150 mg; 0.543 mmol), 2-(4-ethyl-5-oxo-4,5-dihydro-1H-tetrazol-1-yl)ethyl methanesulfonate (141 mg; 0.60 mmol), triethylamine (151 μL; 1.077 mmol), anhydrous potassium carbonate (19 mg; 0.137 mmol), potassium iodide (4 mg; catalytic) and acetonitrile (10 mL) was heated at reflux for about 4 hours. The reaction mixture was filtered and the filtrate was concentrated in vacuo to provide a crude residue which was purified by Preparative HPLC on a Kromasil 100 C18 (250×4.6 mm, 10 μ) column (eluting with acetonitrile/0.01M ammonium acetate (1:1) at a flow rate of 42 mL/min). The title compound eluted at 7.12 min. Acetonitrile was removed by distillation and the remaining aqueous phase was basified to a pH of about 9.0 with a 10% sodium carbonate solution. Standard extractive workup with ethyl acetate gave the compound as a yellow liquid (0.040 g; 18%; unstable), which was used to next step without further purification.

### Step 5

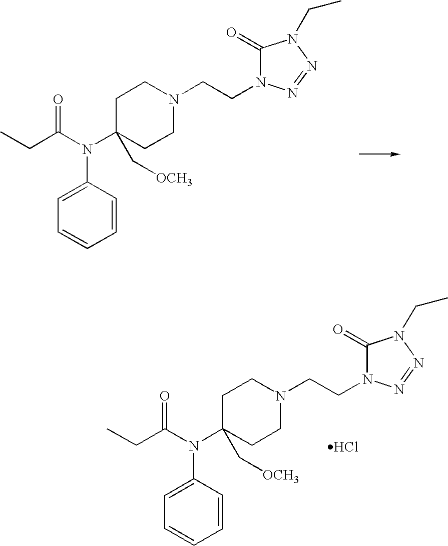

N-[1-(2-(4-Ethyl-5-oxo-4,5-dihydro-1H-tetrazol-1-yl)ethyl)-4-(methoxymethyl)piperidin-4-yl]-N-phenylpropionamide hydrochloride: A saturated solution of hydrochloric gas in diethyl ether was added to a solution of N-[1-(2-(4-ethyl-5-oxo-4,5-dihydro-1H-tetrazol-1-yl)ethyl)-4-(methoxymethyl)piperidin-4-yl]-N-phenylpropionamide (80 mg; 0.192 mmol) in diethyl ether (1.5 mL) until the pH was about 2. The mixture was stirred at ambient temperature for about 30 minutes and then the volatiles were removed in vacuo. The resulting residue was triturated with acetone to yield the title compound as white solid (60 mg; 69%). m.p. 130-133° C., ¹H NMR (400 MHz, pyridine-d₅) δ 0.97 (t, J=7.0 Hz, 3H), 1.2 (t, J=7.4 Hz, 3H), 1.84-1.86 (m, 4H), 2.35-2.39 (m, 4H), 2.61-2.86 (m, 4H), 3.31 (s, 3H), 3.84-3.87 (m, 2H), 4.08-4.16 (m, 4H), 6.5 (br, exchangeable with D₂O, 1H), 7.31-7.35 (m, 5H); IR (KBr) υ 3469, 3415, 3099, 3054, 2986, 2938, 2896, 2822, 2730, 2650, 2550, 2488, 2416, 2366, 1723, 1654, 1593, 1525, 1486, 1452, 1416, 1373, 1305, 1252, 1189, 1145, 1109, 1079, 1023, 969 cm⁻¹; MS 417 (M+1).

### EXAMPLE 8

### N-[1-(2-(4-ethyl-5-oxo-4,5-dihydro-1H-tetrazol-1-yl-1,1,2,2-d₄)ethyl)-4-(methoxymethyl)piperidin-4-yl]-N-phenylpropionamide hydrochloride

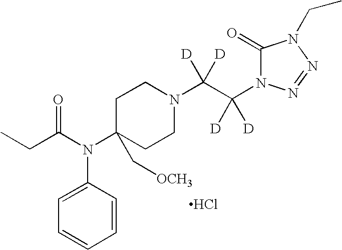

### Step 1

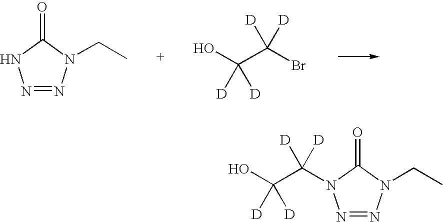

1-Ethyl-4-(2-hydroxyethyl-1,1,2,2-d₄)-1H-tetrazol-5(4H)-one: The procedure of Example 7, Step 2 was followed, but substituting 2-bromoethanol-1,1,2,2-d₄ for 2-bromoethanol. The title compound was isolated as a pale yellow liquid (500 mg; 70%). ¹H NMR (400 MHz, CDCl₃) δ 1.46 (t, J=7.2 Hz, 3H), 2.78 (br, exchangeable with D₂O, 1H), 4.01 (q, J=7.2 Hz, 2H); IR (film) υ 3418, 2987, 1711, 1454, 1407, 1230, 1061, 974, 743 cm⁻¹ MS 163 (M+1).

### Step 2

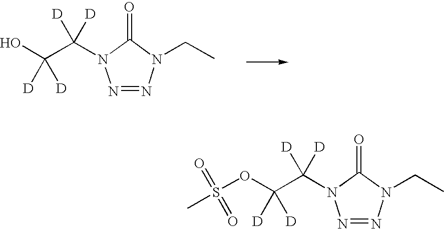

2-(4-Ethyl-5-oxo-4,5-dihydro-1H-tetrazol-1-yl)-1,1,2,2-d₄-ethyl methanesulfonate: The procedure of Example 7, Step 3 was followed, but substituting 1-ethyl-4-(2-hydroxy-1,1,2,2-d₄-ethyl-)-1H-tetrazol-5(4H)-one for 1-ethyl-4-(2-hydroxyethyl)-1H-tetrazol-5 (4H)-one. The title compound was isolated as a pale yellow oil which was used in the next step without further purification (400 mg; 77%). ¹H NMR (400 MHz, CDCl₃) δ 1.45 (t, J=7.4 Hz, 3H), 3.05 (s, 3H), 4.01 (q, J=7.3 Hz, 2H); IR (film) υ 2987, 2938, 1721, 1352, 1179, 981, 855, 815, 738 cm⁻¹; MS 241 (M+1).

### Step 3

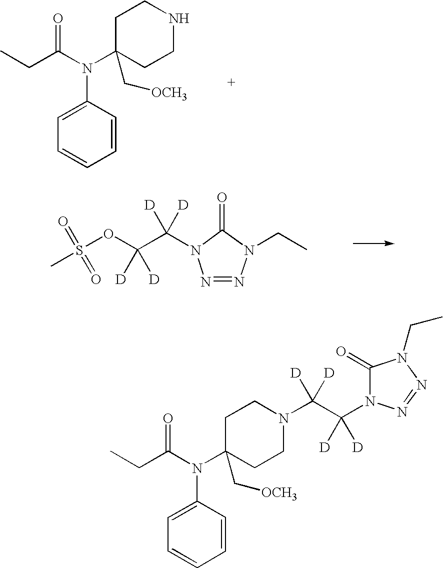

N-[1-(2-(4-Ethyl-5-oxo-4,5-dihydro-1H-tetrazol-1-yl)-1,1,2,2-d₄-ethyl)-4-(methoxymethyl)piperidin-4-yl]-N-phenylpropionamide: The procedure of Example 7, Step 4 was followed, but substituting 2-(4-ethyl-5-oxo-4,5-dihydro-1H-tetrazol-1-yl)-1,1,2,2-d₄-ethyl methanesulfonate for 2-(4-ethyl-5-oxo-4,5-dihydro-1H-tetrazol-1-yl)ethyl methanesulfonate. The title compound was isolated as a yellow liquid (80 mg; 31%, unstable), which was used to next step without further purification.

### Step 4

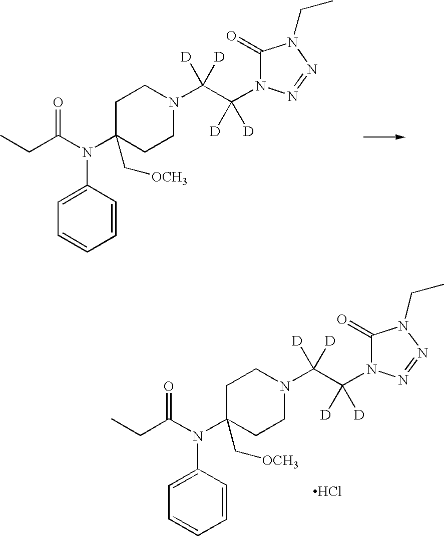

N-[1-(2-(4-Ethyl-5-oxo-4,5-dihydro-1H-tetrazol-1-yl)ethyl-1,1,2,2-d₄)-4-(methoxymethyl)piperidin-4-yl]-N-phenylpropionamide hydrochloride: The procedure of Example 6, Step 5 was followed, but substituting N-[1-(2-(4-ethyl-5-oxo-4,5-dihydro-1H-tetrazol-1-yl)-1,1,2,2-d₄-ethyl)-4-(methoxymethyl)piperidin-4-yl]-N-phenylpropionamide for N-[1-(2-(4-ethyl-5-oxo-4,5-dihydro-1H-tetrazol-1-yl)ethyl)-4-(methoxymethyl)piperidin-4-yl]-N-phenylpropionamide. The title compound was isolated as white solid (55 mg; 64%). m.p. 135-136° C.; ¹H NMR (400 MHz, pyridine-d₅) δ 0.96 (t, J=7.2 Hz, 3H), 1.2 (t, J=7.2 Hz, 3H), 1.81-2.02 (m, 4H), 2.32-2.46 (m, 4H), 2.7-2.85 (m, 2H), 3.30 (s, 3H), 3.85-3.87 (m, 2H) 4.16 (s, 2H), 7.31-7.36 (m, 5H); IR (KBr) υ 3472, 3404, 3289, 3052, 2987, 2936, 2884, 2822, 2650, 2515, 2427, 1721, 1653, 1592, 1488, 1453, 1415, 1373, 1301, 1251, 1182, 1107, 1028, 997 cm⁻¹; MS 421 [(M+1).

### EXAMPLE 9

### N-[1-(2-(4-ethyl-5-oxo-4,5-dihydro-1H-tetrazol-1-yl-1,1,2,2-d₄)ethyl)-4-(methoxy-d₃-methyl)piperidin-4-yl]-N-phenylpropionamide hydrochloride

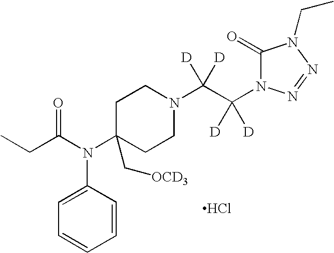

### Step 1

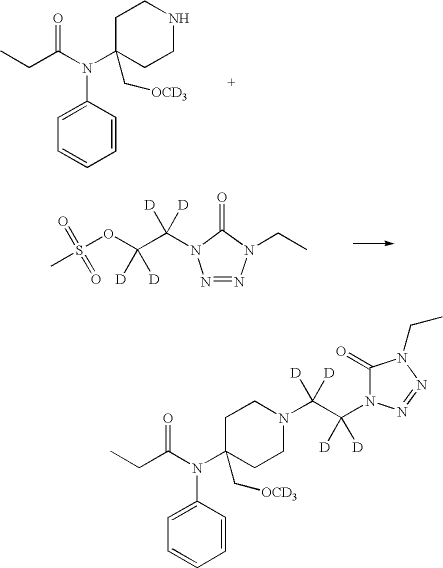

N-[1-(2-(4-Ethyl-5-oxo-4,5-dihydro-1H-tetrazol-1-yl)-1,1,2,2-d₄-ethyl)-4-(d₃-methoxymethyl)piperidin-4-yl]-N-phenylpropionamide: A mixture of N-[4-(d₃-methoxymethyl)piperidin-4-yl]-N-phenylpropionamide (220 mg; 0.787 mmol), 2-(4-ethyl-5-oxo-4,5-dihydro-1H-tetrazol-1-yl)-1,1,2,2-d₄-ethyl methanesulfonate (223 mg; 0.928 mmol), triethylamine (218 μL; 1.55 mmol), anhydrous potassium carbonate (25 mg; 0.181 mmol), potassium iodide (5 mg; catalytic) and acetonitrile (10 mL) was heated at reflux for about 5 hours. The mixture was filtered and the filtrate was concentrated in vacuo to provide a crude residue which was purified by Preparative HPLC on an X-Terra RP-8 (250×19 mm, 10 μ) column (eluting with acetonitrile/0.01M ammonium bicarbonate (gradient) at a flow rate of 42 mL/min). The title compound eluted at 8.39 min. Acetonitrile was removed by distillation and the remaining aqueous phase was basified to a pH of about 9.0 with a 10% sodium carbonate solution. Standard extractive workup with ethyl acetate gave the title compound as a yellow liquid (75 mg; 22%; unstable), which was used to next step without further purification.

### Step 2

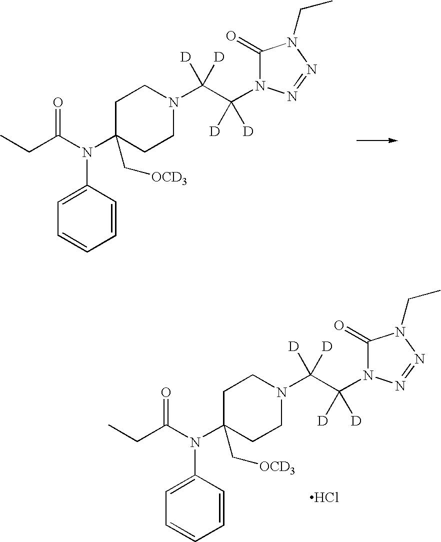

N-[1-(2-(4-Ethyl-5-oxo-4,5-dihydro-1H-tetrazol-1-yl)-1,1,2,2-d₄-ethyl)-4-(methoxy-d₃-methyl)piperidin-4-yl]-N-phenylpropionamide hydrochloride: The procedure of Example 7, Step 5 was followed, but substituting N-[1-(2-(4-Ethyl-5-oxo-4,5-dihydro-1H-tetrazol-1-yl)1,1,2,2-d₄-ethyl)-4-(d₃-methoxymethyl)piperidin-4-yl]-N-phenylpropionamide for N-[1-(2-(4-ethyl-5-oxo-4,5-dihydro-1H-tetrazol-1-yl)ethyl)-4-(methoxymethyl)piperidin-4-yl]-N-phenylpropionamide. The title compound was isolated as a white solid (50 mg; 61%). m.p. 128-131° C.; ¹H NMR (400 MHz, pyridine-d₅) δ 0.96 (t, J=7.2 Hz, 3H), 1.2 (t, J=7.2 Hz, 3H), 1.83-1.85 (m, 4H), 2.37-2.40 (m, 4H), 2.75-2.81 (m, 2H), 3.85-3.86 (m, 2H), 4.15 (s, 3H), 7.30-7.35 (m, 5H); IR (KBr) υ 3470, 3416, 3099, 3052, 2988, 2939, 2883, 2648, 2507, 2424, 2366, 2246, 2200, 2125, 2058, 1722, 1654, 1593, 1486, 1452, 1415, 1374, 1300, 1252, 1185, 1129, 1075, 1024, 991 cm⁻¹; MS 424 [(M+1).

### EXAMPLE 10

### d₂₃-N-(4-Ethyl-piperidin-4-yl)-N-phenyl-propionamide (d₂₃-norsulfentanil or d₂₃-noralfentanil)

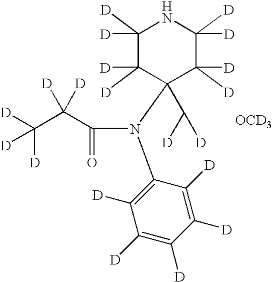

### Step 1

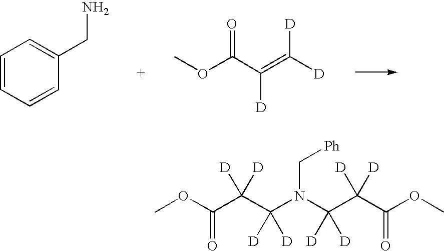

d₈-3-[Benzyl-(2-methoxycarbonyl-ethyl)-amino]-propionic acid methyl ester: The procedure of Step 1 is carried out using the methods described by Lee et al., Bioorganic & Medicinal Chemistry Letters 17 2007, 2161-2165. At ambient temperature, benzylamine is added to a solution of d₃-methylpropenoate (available commercially from Medical Isotopes Inc., Pelham, N.H. 03076) in d₄-methanol (available commercially from Sigma-Aldrich, St. Louis Mo. 63103). The mixture is heated at reflux for about 4 hours. Standard extractive workup affords the title product.

### Step 2

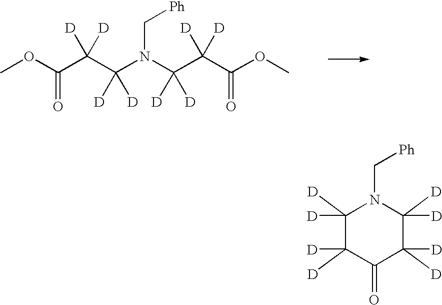

d₈-1-Benzyl-piperidin-4-one: The procedure of Step 2 is carried out using the methods described by Lee et al., Bioorganic & Medicinal Chemistry Letters 172007, 2161-2165. d₈-3-[Benzyl-(2-methoxycarbonyl-ethyl)-amino]-propionic acid methyl ester is added to a suspension of sodium in toluene. The mixture is heated at reflux for about 2 hours and then cooled to about 0° C. Deuterium chloride is added, the mixture is heated at an elevated temperature for about 3 to 4 hours, and then standard extractive workup is performed to give the title product.

### Step 3

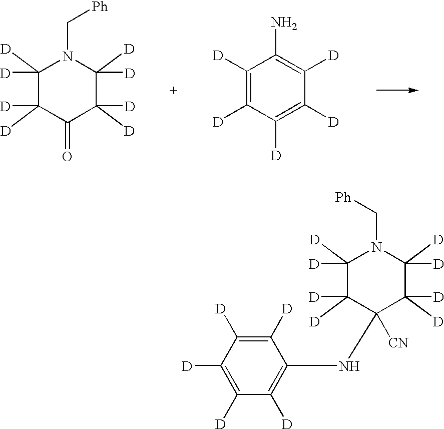

d₁₃-1-Benzyl-4-phenylamino-piperidine-4-carbonitrile: The procedure of step 3 is carried out using the methods described in WO 2008/005423 A1. At about 5° C., acetic acid is added drop wise to a mixture d₈-1-benzyl-piperidin-4-one, d₅-aniline (available commercially from Sigma-Aldrich, St. Louis Mo. 63103), potassium cyanide, and dichloromethane. The mixture, over a period of 1 hour, is gradually warmed to about 50° C., and then maintained at that temperature for about 17 hours. After cooling to about 20° C., crushed ice is added. Following basification, standard extractive workup is performed with dichloromethane to give light brown slurry. The slurry is suspended in isopropanol, and cooled to about 0° C. The resulting precipitate is collected by filtration, washed and dried in vacuo to yield the title product.

### Step 4

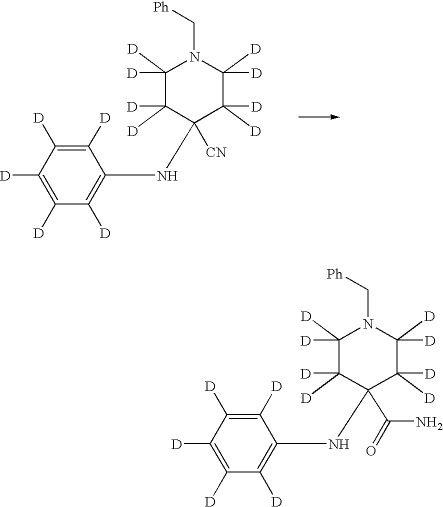

d₁₃-1-Benzyl-4-phenylamino-piperidine-4-carboxylic acid amide: The procedure of Step 4 is carried out using the methods described in WO 2008/005423 A1. At about 25-30° C., d₁₃-1-benzyl-4-phenylamino-piperidine-4-carbonitrile is added to a solution of d₂-sulfuric acid in deuterium oxide. The mixture is maintained at 25-30° C. for about 20 hours, and then mixed with crushed ice. Following 30 min at about 0° C., the resulting precipitate is collected by filtration, and dissolved in water. The solution is basified and the resulting precipitate collected by filtration. The solids are then dried in a vacuum oven to afford the title product.

### Step 5

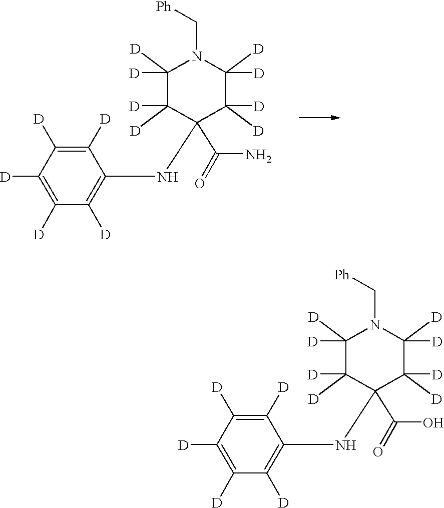

d₁₃-1-Benzyl-4phenylamino-piperidine-4-carboxylic acid: The procedure of step 5 is carried out using the methods described in WO 2008/005423 A1. A mixture of d₁₃-1-benzyl-4-phenylamino-piperidine-4-carboxylic acid amide, potassium hydroxide, and 1,2-propanediol is heated at reflux for about 27 hours. After cooling to ambient temperature, water is added and the mixture is slowly cooled to about 5 to 10° C. The mixture is acidified with concentrated hydrochloric acid, and then basified with sodium hydroxide. The resulting precipitate is collected and recrystallized from isopropanol-water to afford to title product.

### Step 6

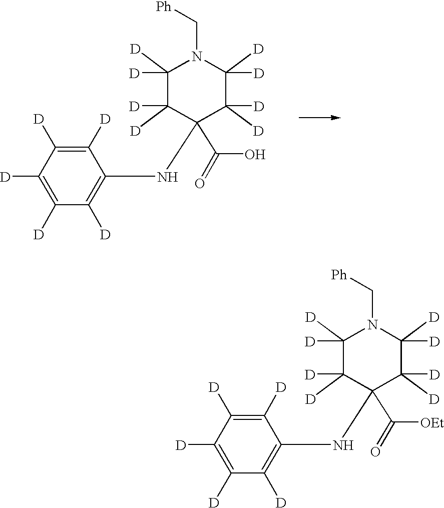

d₁₃-1-Benzyl-4-phenylamino-piperidine-4-carboxylic acid ethyl ester: The procedure of Step 6 is carried out using the methods described by Colapret et al., Journal of Medicinal Chemistry 1989, 32, 968-974. A slurry of d₁₃-1-benzyl-4-phenylamino-piperidine-4-carboxylic acid in a mixture of ethanol and toluene (70:30) is heated at reflux for about 10 days while the azeotrope is collected via a Dean-Stark trap. The mixture is cooled to about 0° C., and a 10% sodium hydroxide solution is added dropwise. Standard extractive workup gives a crude residue which is purified by flash column chromatography to afford the title product.

### Step 7

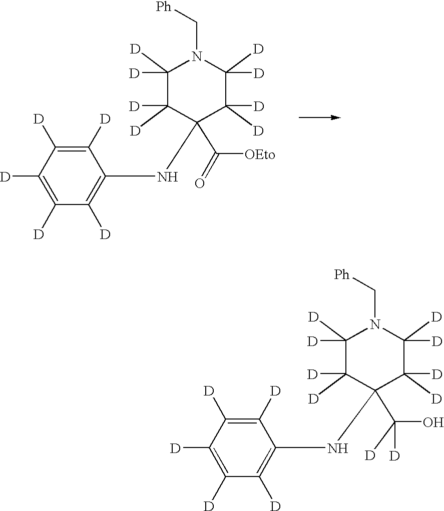

d₁₅-(1-Benzyl-4-phenylamino-piperidin-4-yl)-methanol: The procedure of Step 7 is carried out using the methods described by Colapret et al., Journal of Medicinal Chemistry 1989, 32, 968-974. At about 0° C., d₁₃-1-benzyl-4-phenylamino-piperidine-4-carboxylic acid ethyl ester is added to a slurry of lithium aluminum deuteride in dry tetrahydrofuran. The mixture is maintained at about 56° C. for about 12 hours. After cooling to ambient temperature, the reaction is quenched with water. Standard extractive workup gives a crude residue which is purified by flash column chromatography to afford the title product.

### Step 8

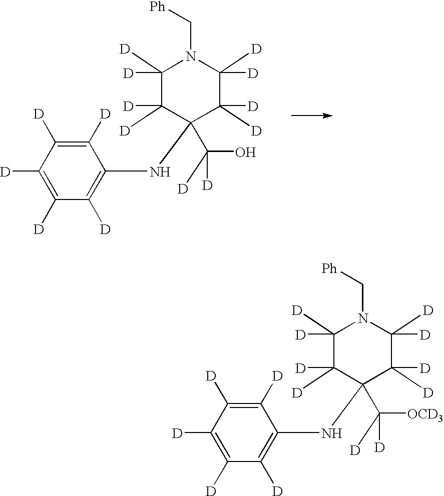

d₁₈-(1-Benzyl-4-methoxymethyl-piperidin-4-yl)-phenyl-amine: The procedure of step 8 is carried out using the methods described in WO 2008/005423 A1. Under a nitrogen atmosphere and at ambient temperature, d₁₅-(1-benzyl-4-phenylamino-piperidin-4-yl)-methanol in tetrahydrofuran is added dropwise to a suspension of sodium hydride in tetrahydrofuran. The suspension is maintained at ambient temperature for about 2 hours. d₃-Iodomethane (available commercially from Sigma-Aldrich, St. Louis Mo. 63103) is added dropwise, and the reaction is maintained at ambient temperature for about 3 hours. The reaction is quenched with water, and standard extractive work up gives the title product.

### Step 9

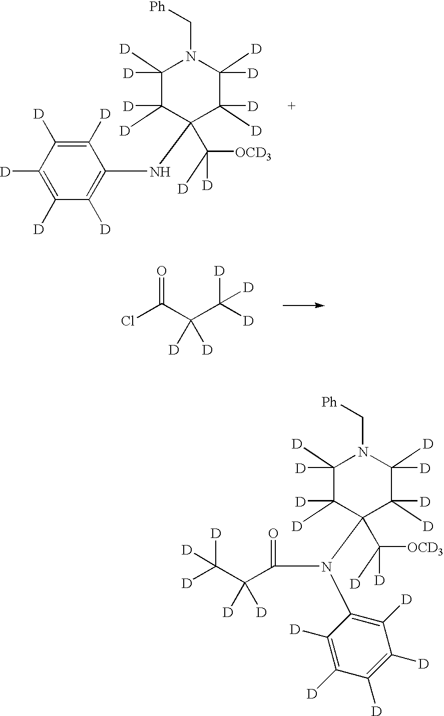

d₂₃-N-(1-Benzyl-4-methoxymethyl-piperidin-4-yl)-N-phenyl-propionamide: The procedure of step 9 is carried out using the methods described in WO 2008/005423 A1. At a temperature below 26° C., d₅-propionyl chloride is added dropwise to a mixture containing d₁₈-(1-benzyl-4-methoxymethyl-piperidin-4-yl)-phenyl-amine and dichloromethane. The mixture is maintained at ambient temperature for about 3 hours, triethylamine is added, and the mixture is maintained at ambient temperature for an additional hour. Standard extractive workup affords the title product.

### Step 10

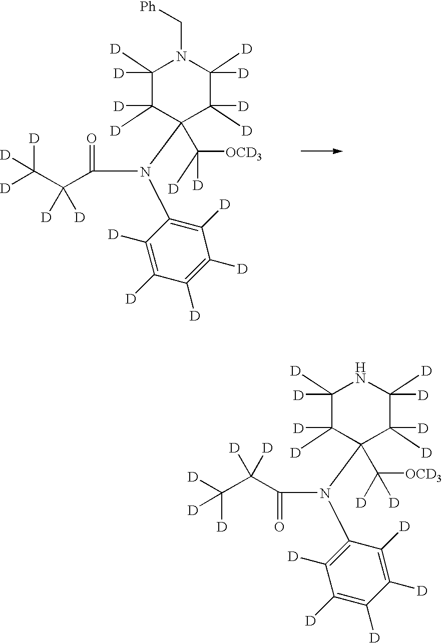

d₂₃-N-(4-Ethyl-piperidin-4-yl)-N-phenyl-propionamide (d₂₃-Norsulfentanil or d₂₃-Noralfentanil): The procedure of step 10 is carried out using the methods described in WO 2008/005423 A1. In a Parr hydrogenation bottle, a mixture of d₂₃-N-(1-benzyl-4-methoxymethyl-piperidin-4-yl)-N-phenyl-propionamide, deuterium oxide, d₄-acetic acid, deuterium chloride, and 10% palladium on carbon (wet paste) is flushed with hydrogen (3 times), and then stirred at 60 psi while warming the contents of the bottle to about 50° C. over a period of about 1 hour. The mixture is cooled to ambient temperature and filtered through a celite pad. The solids are washed with water, and the wash is combined with the filtrate. Standard extractive workup gives the title compound.

### EXAMPLE 11

### d₃₀-N-[4-(methoxymethyl)-1-(2-thiophen-2-ylethyl)-4-piperidyl]-N-phenyl propanamide (d₃₀-sufentanil)

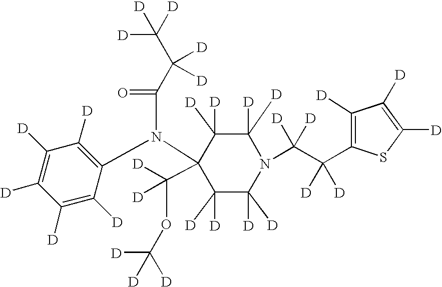

### Step 1

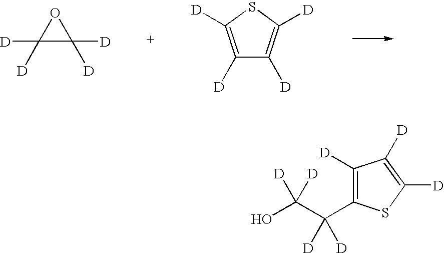

d₇-2-Thiophen-2-yl-ethanol: The procedure of Step 1 is carried out using the methods described in U.S. Pat. No. 6,639,083. Under nitrogen, dry toluene and oleic acid are added to cut pieces of sodium. The suspension is heated to about 102° C. for about 30 minutes and then cooled to ambient temperature. At about 0° C. and under nitrogen, the sodium suspension is then added to a jacketed reactor containing d₄-thiophene (available commercially from C/D/N Isotopes Inc., Pointe-Claire, Quebec, Canada H9R 1H1), 2,3-dimethyl-1,3-butadiene, and tetrahydrofuran. The suspension is maintained at about 0° C. for about 2 hours. The temperature is then warmed to about 20° C., and d₄-ethylene oxide (available commercially from Sigma-Aldrich, St. Louis Mo. 63103) is added. The suspension is maintained at about 20° C. for about 20 minutes, and then slowly transferred to a reactor, with a nitrogen atmosphere, containing ice, while maintaining the temperature at about 13° C. Standard extractive workup gives the title product.

### Step 2

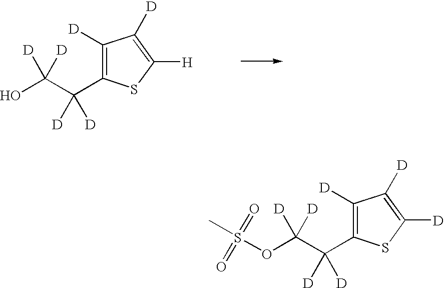

d₇-2(Thien-2-yl)ethyl methanesulfonate: The procedure of Step 2 is carried out using the methods described by WO 2008/005423 A1. At about 10° C., methanesulfonyl chloride is slowly added (over a period of about 1 hour) to a solution of d₇-2-thiophen-2-yl-ethanol and triethylamine in methylene chloride. The mixture is maintained at ambient temperature for about 16 hours and then quenched with water. Standard extractive workup gives the title product.

### Step 3

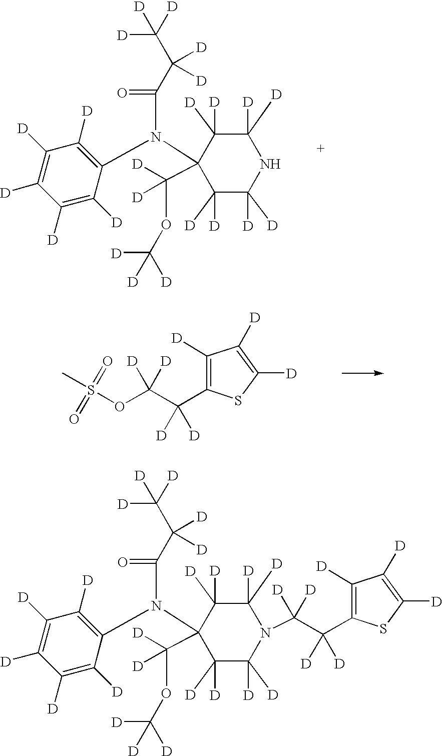

d₃₀-N-[4-(methoxymethyl)-1-(2-thiophen-2-ylethyl)-4-piperidyl]-N-phenyl propanamide: The procedure of step 3 is carried out using the methods described in WO 2008/005423 A1. Triethylamine, potassium iodide and potassium carbonate are added to a solution of d₇-2(thien-2-yl)ethyl methanesulfonate and d₂₃-N-(4-Ethyl-piperidin-4-yl)-N-phenyl-propionamide in acetonitrile. The mixture is heated at reflux for about 5 hours, and then cooled to ambient temperature. The mixture is then poured onto crushed ice and maintained at about 0° C. for about 1 hour. The precipitate is collected by filtration, and washed with an acetonitrile-water mixture (1:4). The precipitate is dried and recrystallized from an isopropanol-water mixture (1:1), to give the title compound.

### EXAMPLE 12

d₃₂-N-[1-[2-(4-ethyl-5-oxo-1,4-dihydrotetrazol-1-yl)ethyl]-4-(methoxymethyl)-4-piperidyl]-N-phenyl-propanamide (d₃₂-alfentanil)

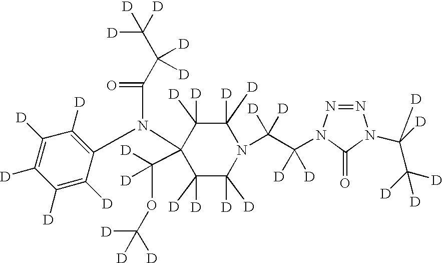

### Step 1

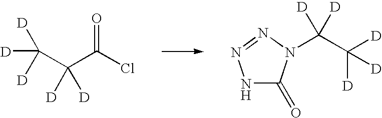

d₅-1-Ethyltetrazolinone: The procedure of Step 1 is carried out using the methods described in Janssens et al., Journal of Medicinal Chemistry 1986, 29(11), 2290-7. A solution of aluminum chloride in tetrahydrofuran is added to a suspension of sodium azide in dry tetrahydrofuran, and heated at reflux for about 1 hour. The mixture is cooled to ambient temperature and a solution of d₅-propionyl chloride (available commercially from C/D/N Isotopes Inc., Pointe-Claire, Quebec, Canada H9R 1H1) in tetrahydrofuran is added. The mixture is then heated at gentle reflux for about 24 hours. Standard extractive workup gives the title product as a tan solid.

### Step 2

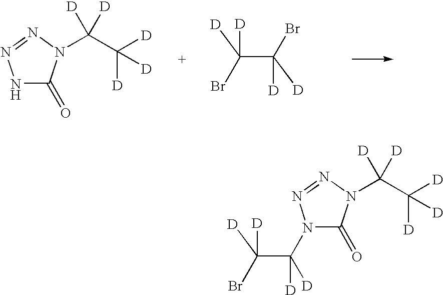

d₉-1-(2-Bromo-ethyl)-4-ethyl-1,4-dihydro-tetrazol-5-one: The procedure of Step 2 is carried out using the methods described by WO 2001/40184 A2. A mixture of d₅-1-ethyltetrazolinone, d₄-1,2-dibromoethane (available commercially from Sigma-Aldrich, St. Louis Mo. 63103), triethylamine, and acetonitrile is heated at reflux for about 1 hour. The resulting oil is taken up in isobutyl alcohol. Azeotropic distillation affords the title product.

### Step 3

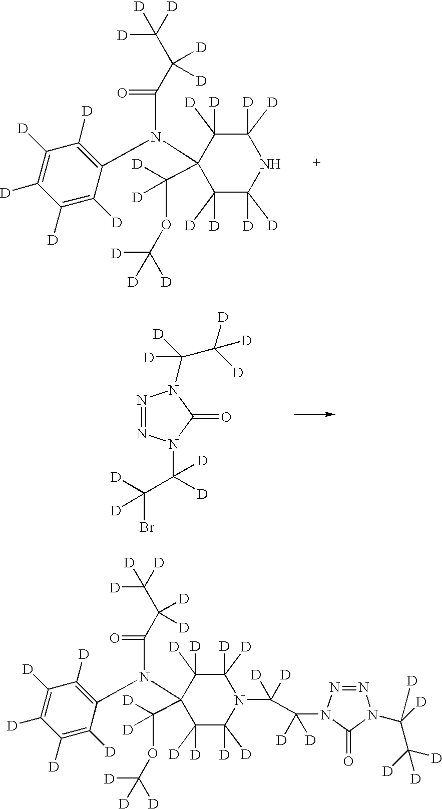

d₃₂-N-[1-[2-(4-ethyl-5-oxo-1,4-dihydrotetrazol-1-yl)ethyl]-4-(methoxymethyl)-4-piperidyl]-N-phenyl-propanamide: The procedure of step 3 is carried out using the methods described in Janssens et al., Journal of Medicinal Chemistry 1986, 29(11), 2290-7. A suspension of d₉-1-(2-bromo-ethyl)-4-ethyl-1,4-dihydro-tetrazol-5-one, d₂₃-N-(4-ethyl-piperidin-4-yl)-N-phenyl-propionamide, sodium carbonate, and potassium iodide in 4-methyl-2-pentanone is heated at reflux for about 18 hours. After cooling to ambient temperature, water is added. Standard extractive workup gives the title compound.

### EXAMPLE 13

### d₂₈-N-(1-Phenethyl-piperidin-4-yl)-N-phenyl-propionamide

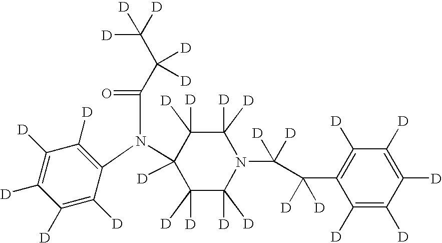

### Step 1

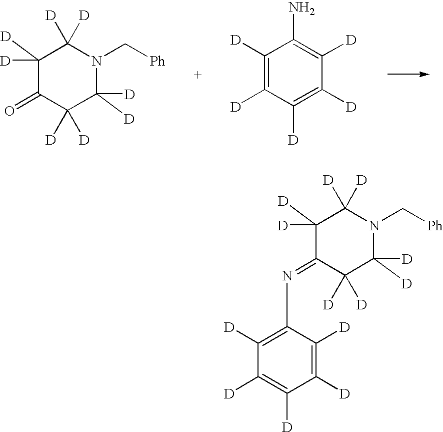

d₁₃-(1-Benzyl-piperidin-4-ylidene)-phenyl-amine: The procedure of Step 1 is carried out using the methods described in U.S. Pat. No. 3,164,600. A mixture of d₈-1-benzylpiperidone, d₅-aniline, and toluenesulfonic acid in toluene is heated at reflux until the calculated amount of water is separated. The solvent is removed in vacuo. The resulting residue is taken up in ether, filtered, and distilled to give the title product.

### Step 2

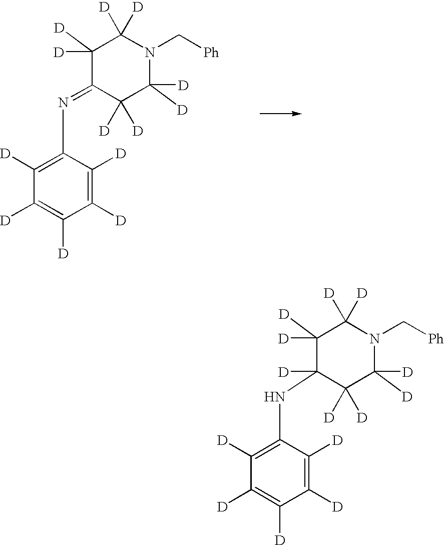

d₁₄-(1-Benzyl-piperidin-4-yl)-phenyl-amine: The procedure of Step 2 is carried out using the methods described in U.S. Pat. No. 3,164,600. d₁₃-(1-Benzyl-piperidin-4-ylidene)-phenyl-amine in ether is added to a suspension of lithium aluminum deuteride in ether, and then heated at reflux for about 5 hours. Standard extractive workup gives a crude residue which is recrystallized from petroleum ether to give the title product.

### Step 3

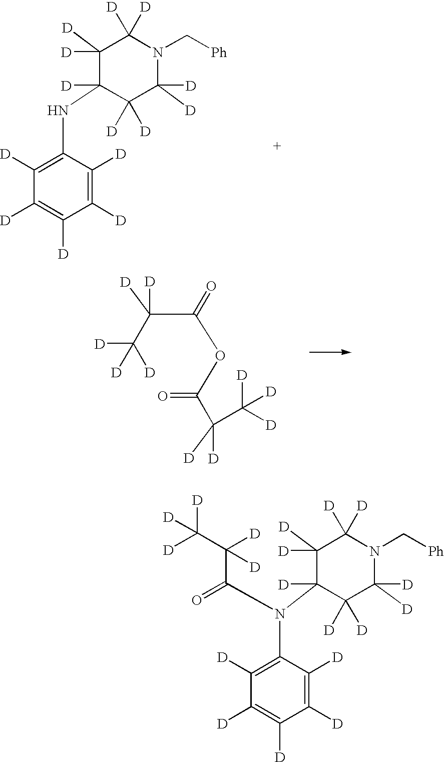

d₁₉-N-(1-Benzyl-piperidin-4-yl)-N-phenyl-propionamide: The procedure of Step 3 is carried out using the methods described in U.S. Pat. No. 3,164,600. d₁₄-(1-Benzyl-piperidin-4-yl)-phenyl-amine is added to d₁₀-propionic anhydride in toluene. The mixture is heated at reflux for about 7 hours. Standard extractive gives a crude residue which is recrystallized from petroleum ether to give the title product.

### Step 4

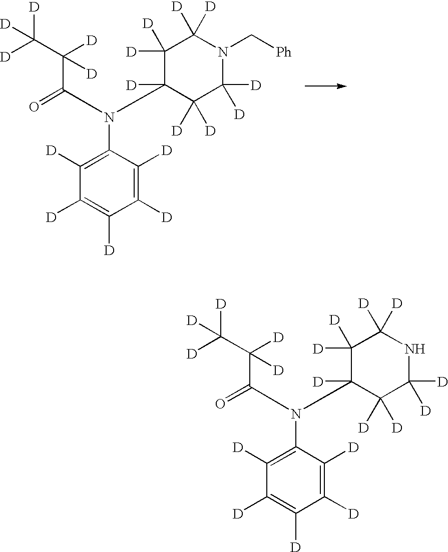

d₁₉-N-Phenyl-N-piperidin-4-yl-propionamide: The procedure of Step 4 is carried out using the methods described in U.S. Pat. No. 3,164,600. Under an atmosphere of hydrogen, d₁₉-N-(1-benzyl-piperidin-4-yl)-N-phenyl-propionamide, ethanol, and 10% palladium on carbon is heated at ambient temperature, until reaction is complete. The mixture is filtered and the solvent is removed to give the title product.

### Step 5

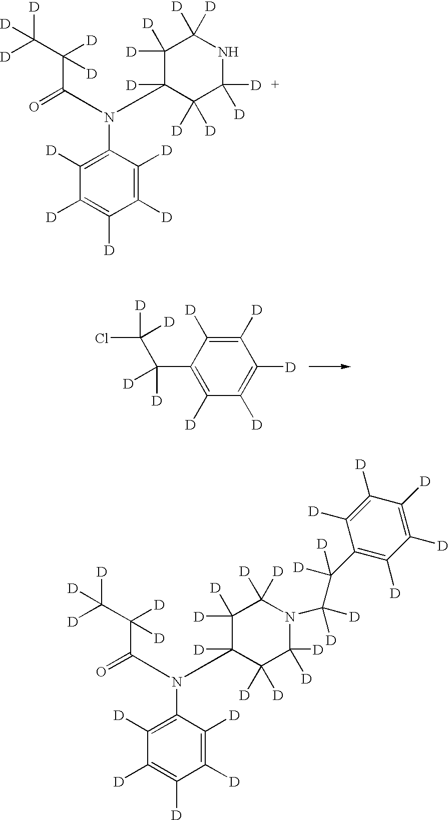

d₂₈-N-(1-Phenethyl-piperidin-4-yl)-N-phenyl-propionamide: The procedure of Step 5 is carried out using the methods described in U.S. Pat. No. 3,164,600. A mixture of d₁₉-N-Phenyl-N-piperidin-4-yl-propionamide, d₉-(2-chloroethyl)-benzene, sodium carbonate, and potassium iodide in 4-methyl-2-pentanone is heated at reflux for about 36 hours. Standard extractive work up gives the title compound.

The following compounds can generally be made using the methods described above. It is expected that these compounds when made will have activity similar to those described in the examples above:

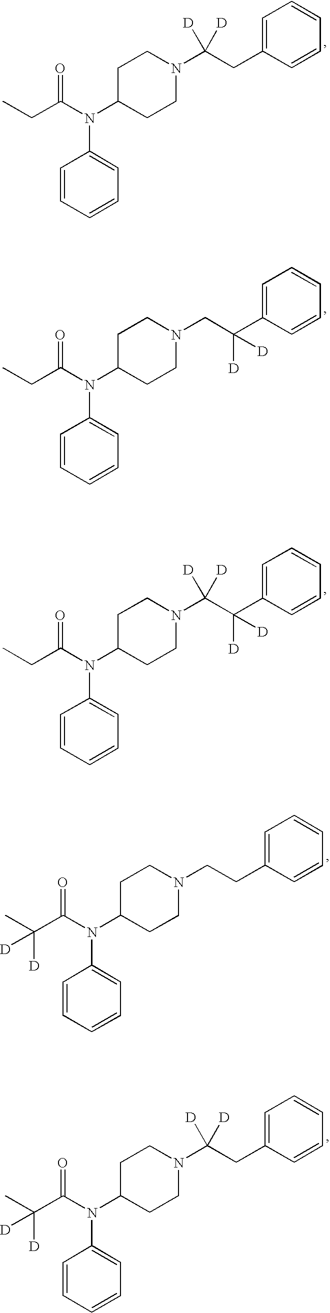

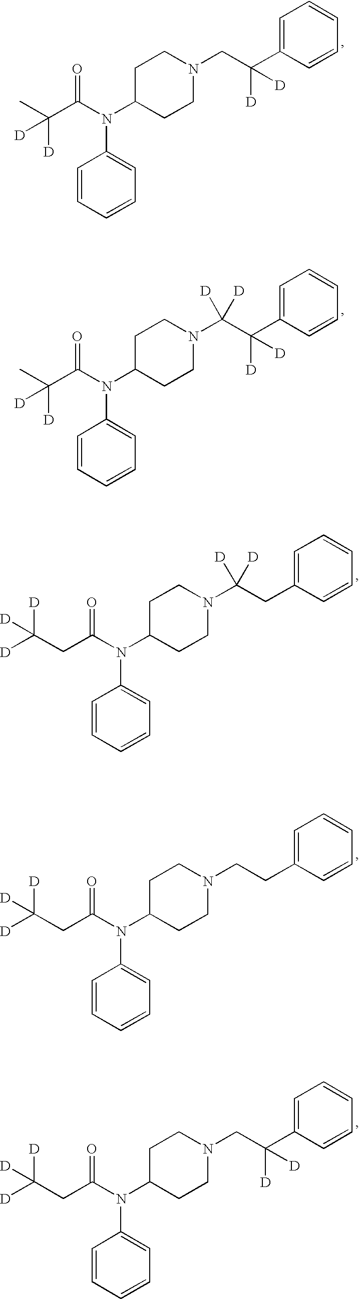

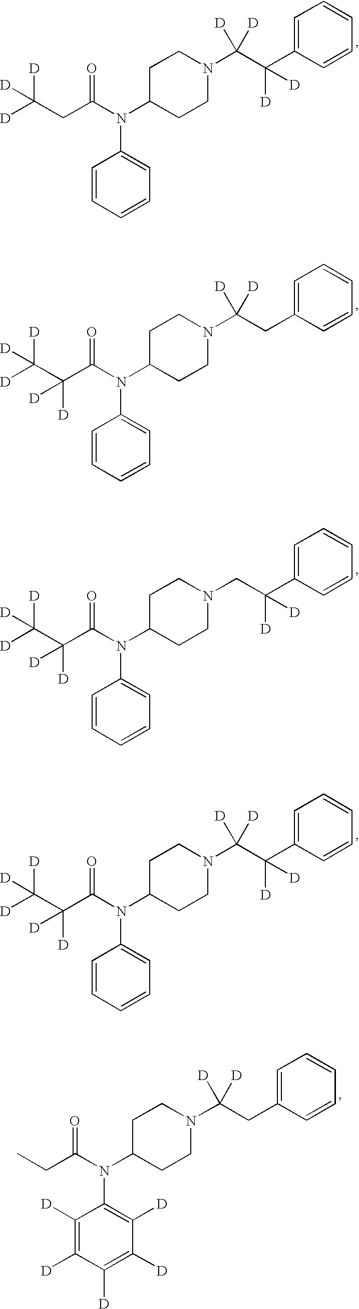

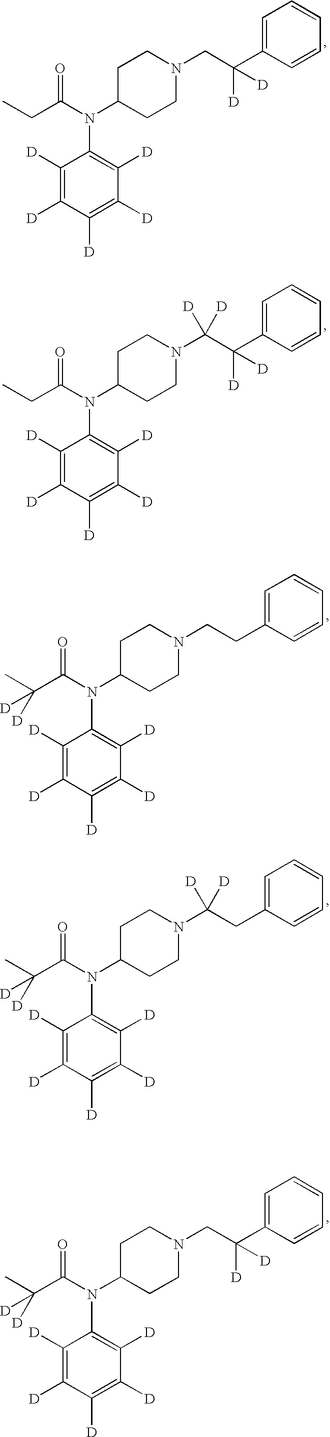

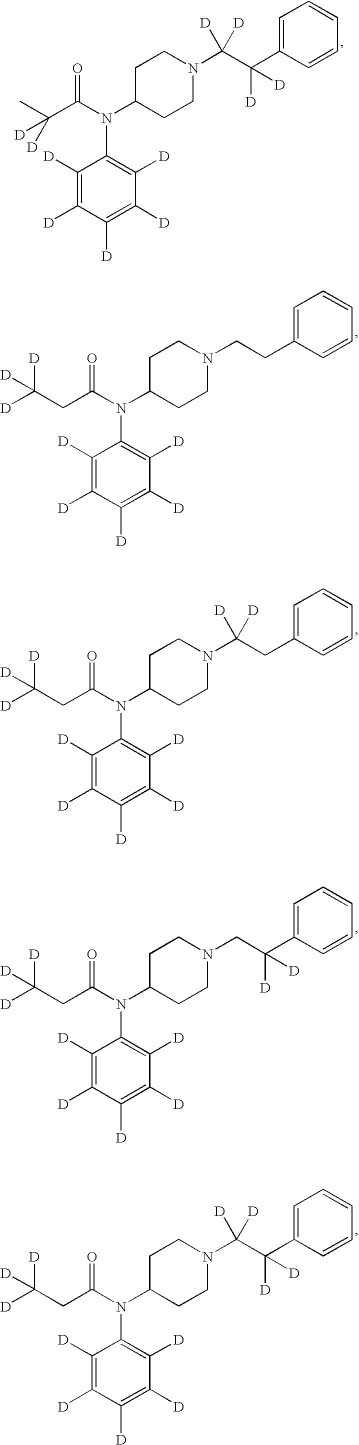

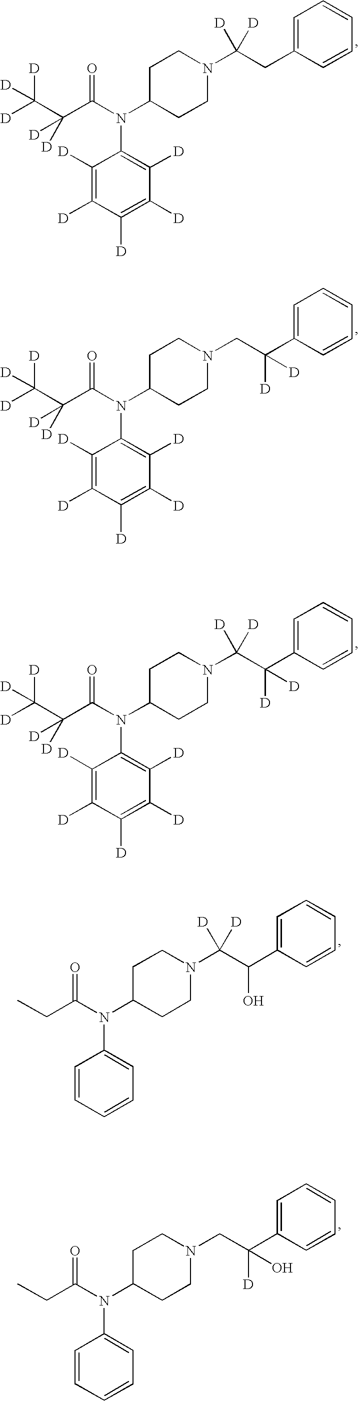

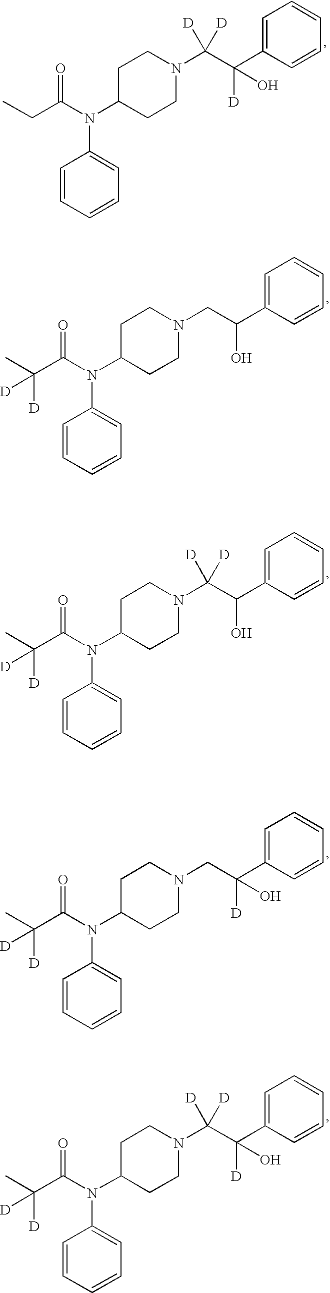

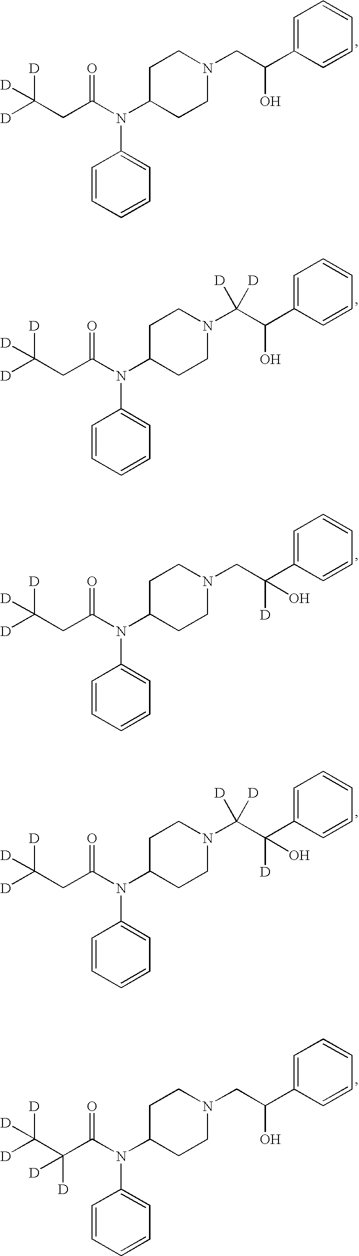

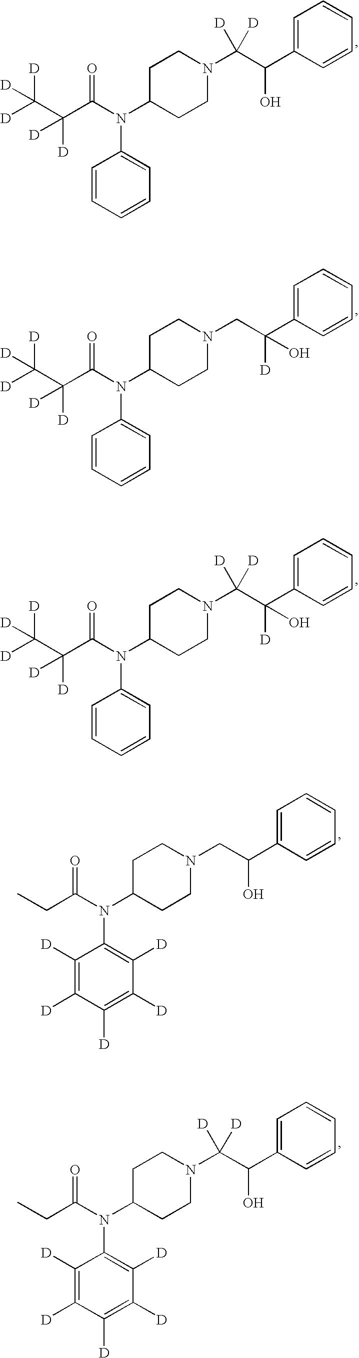

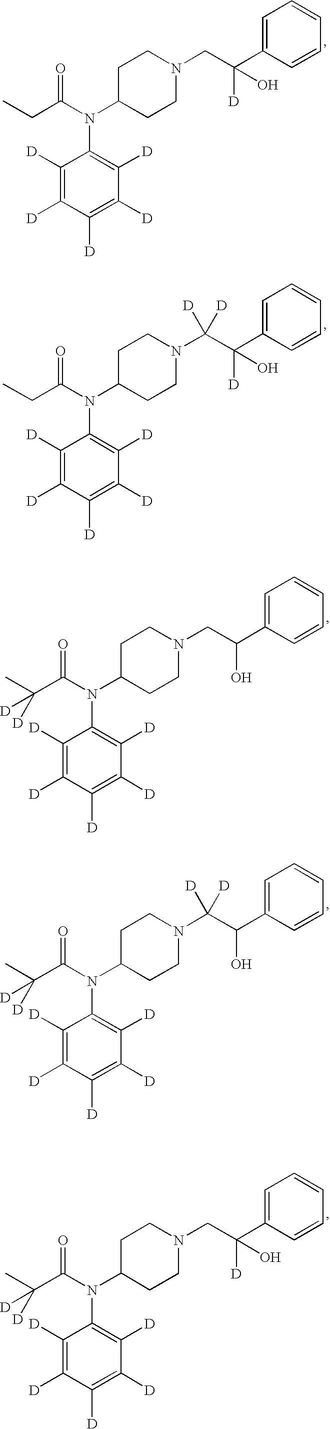

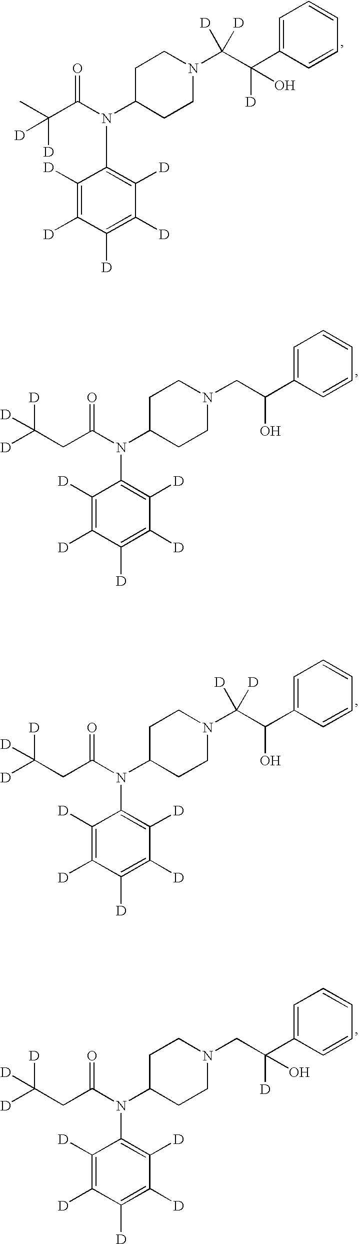

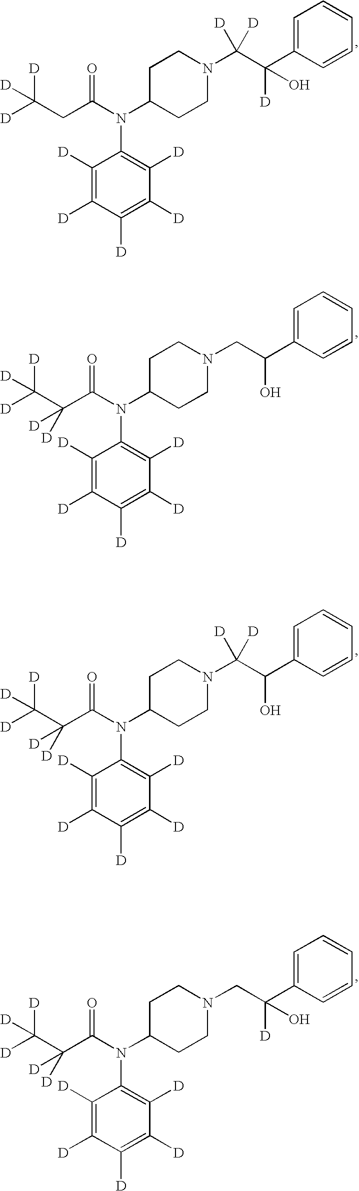

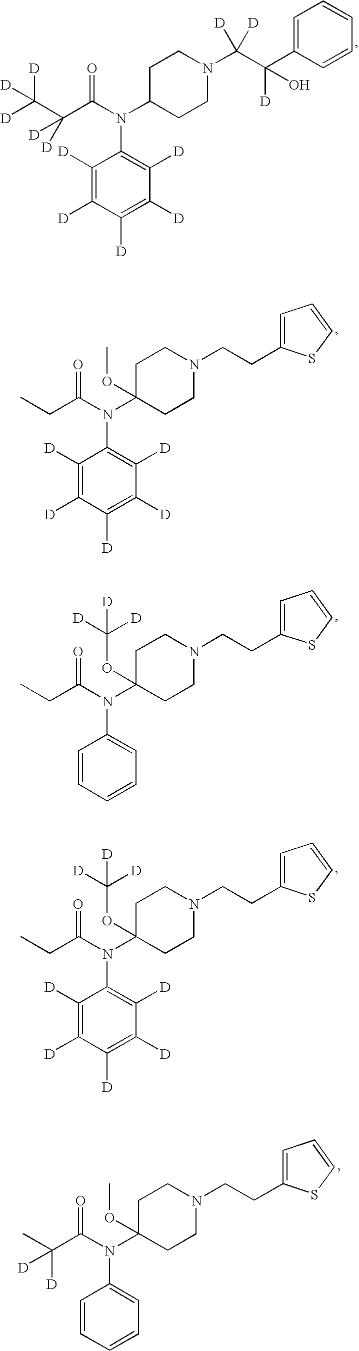

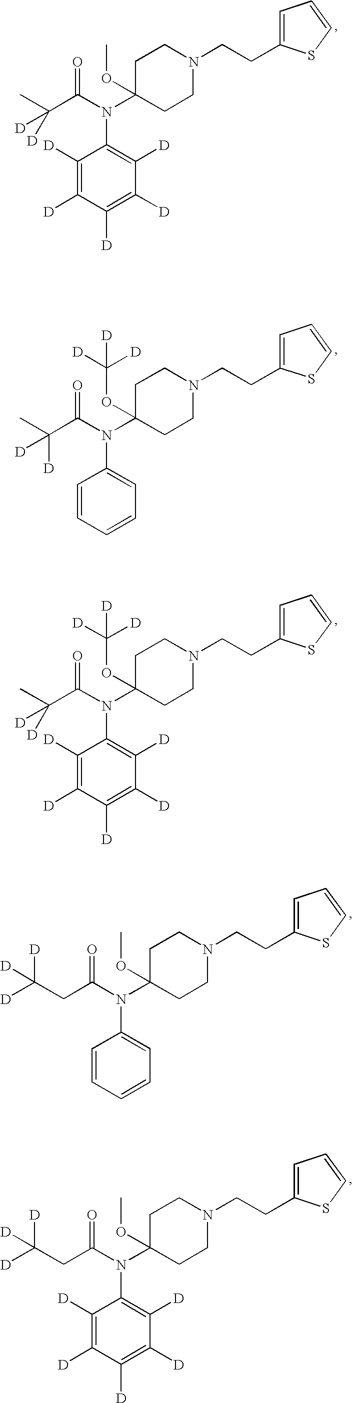

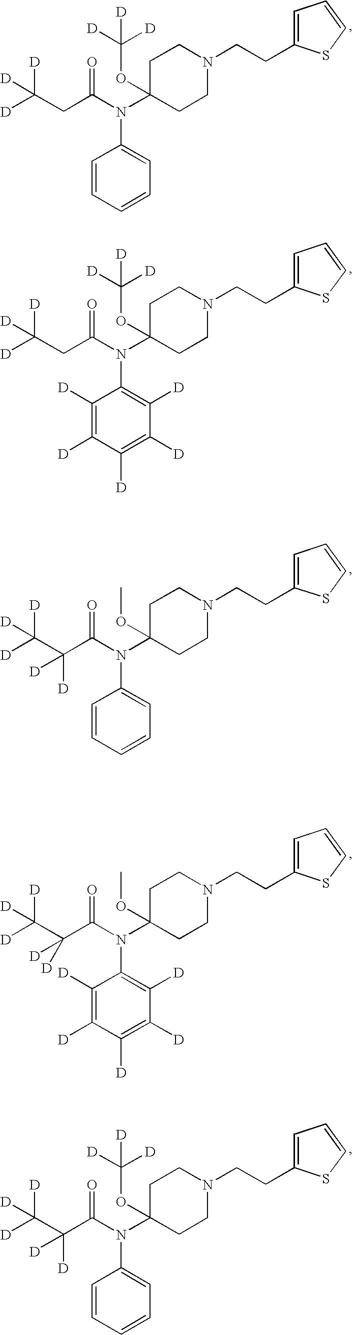

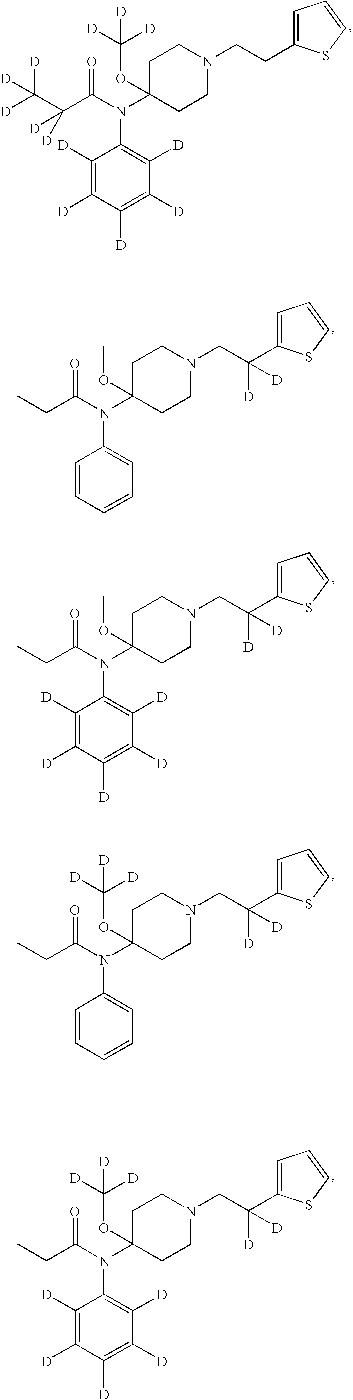

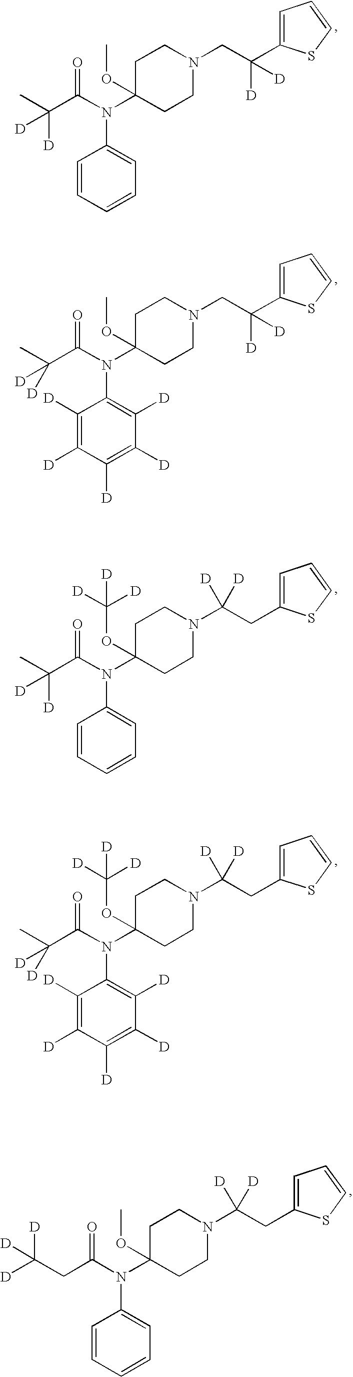

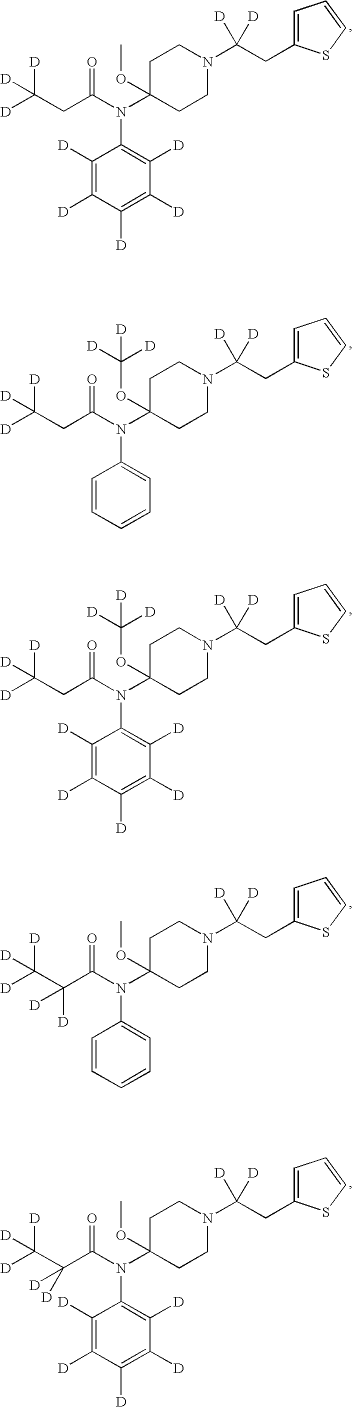

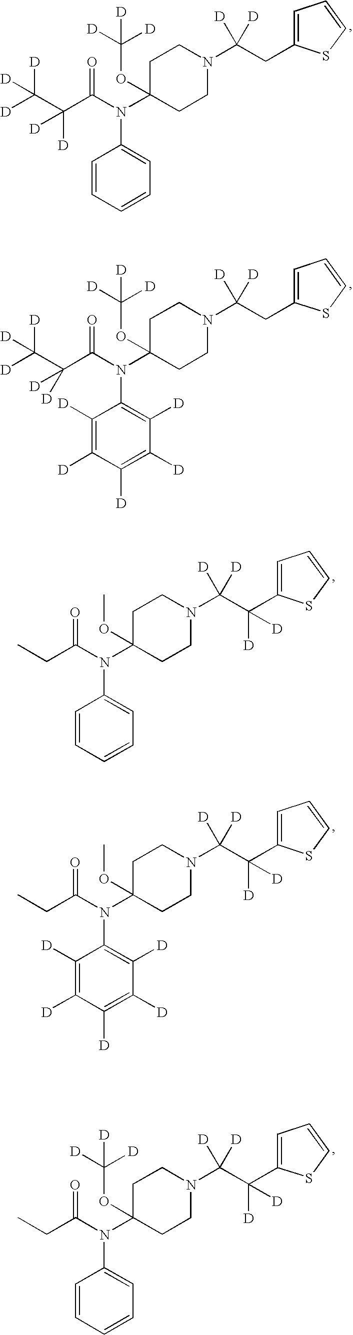

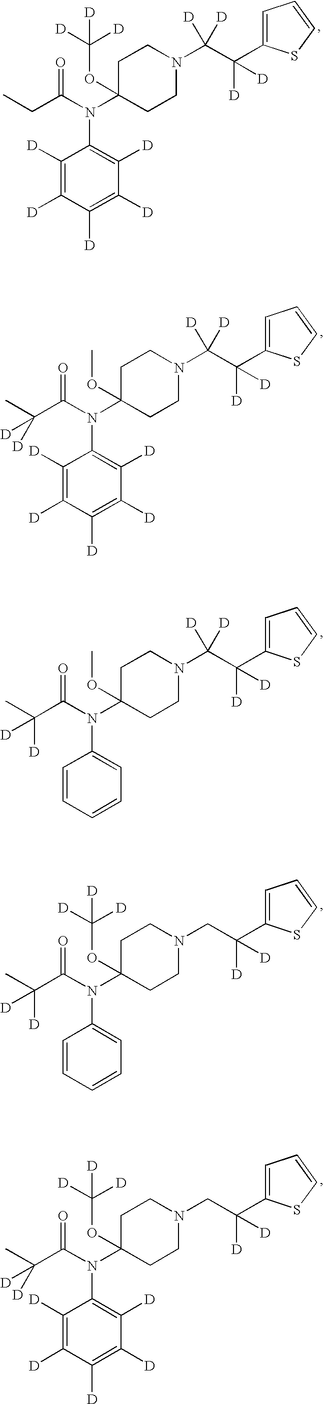

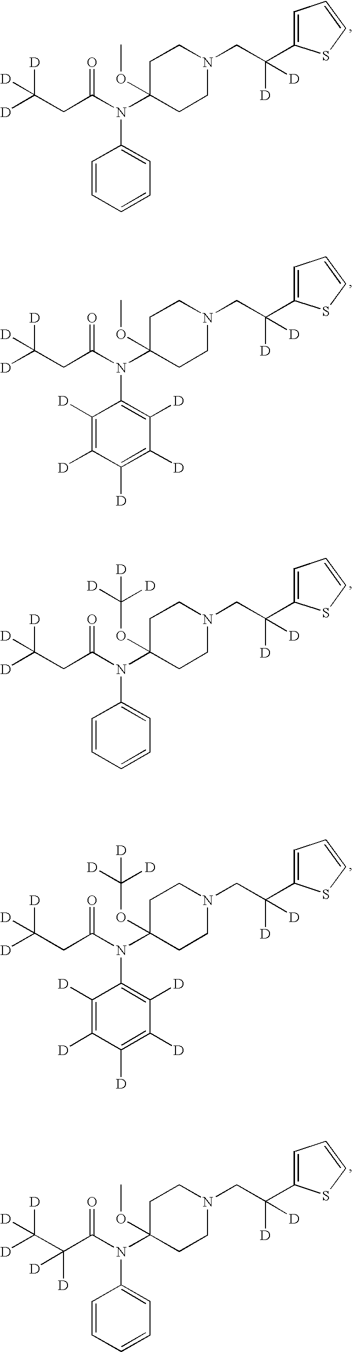

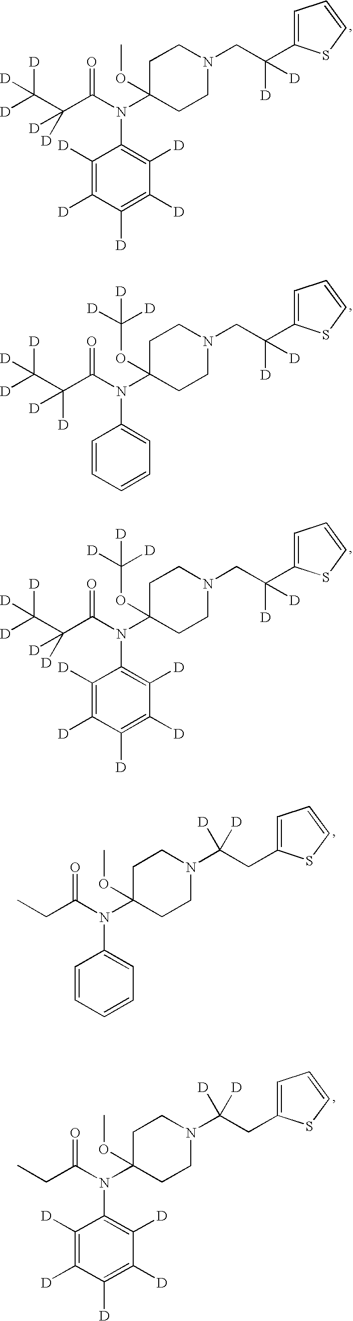

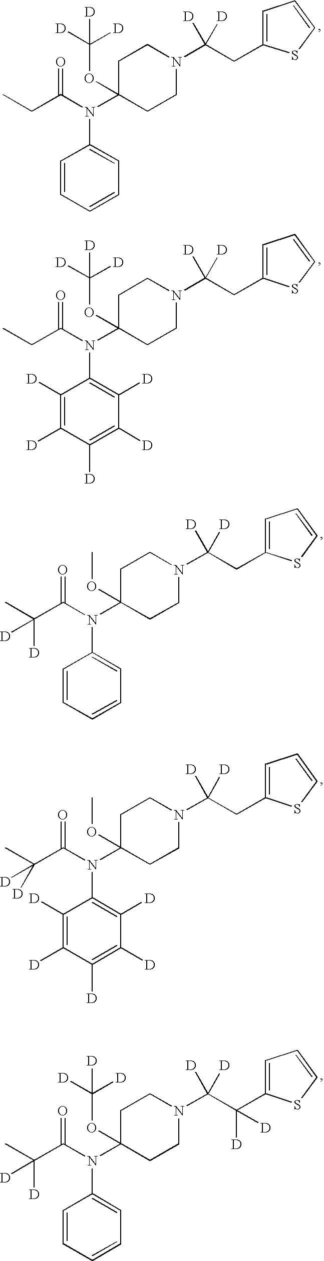

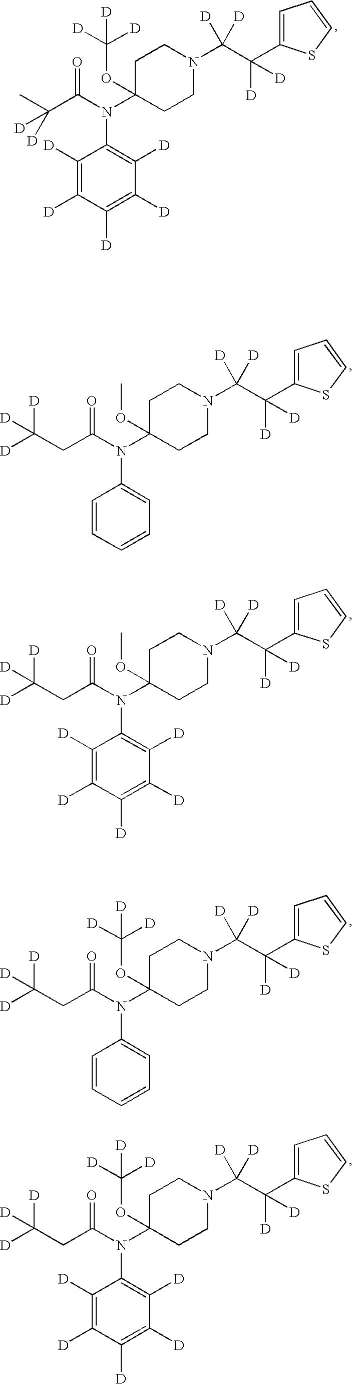

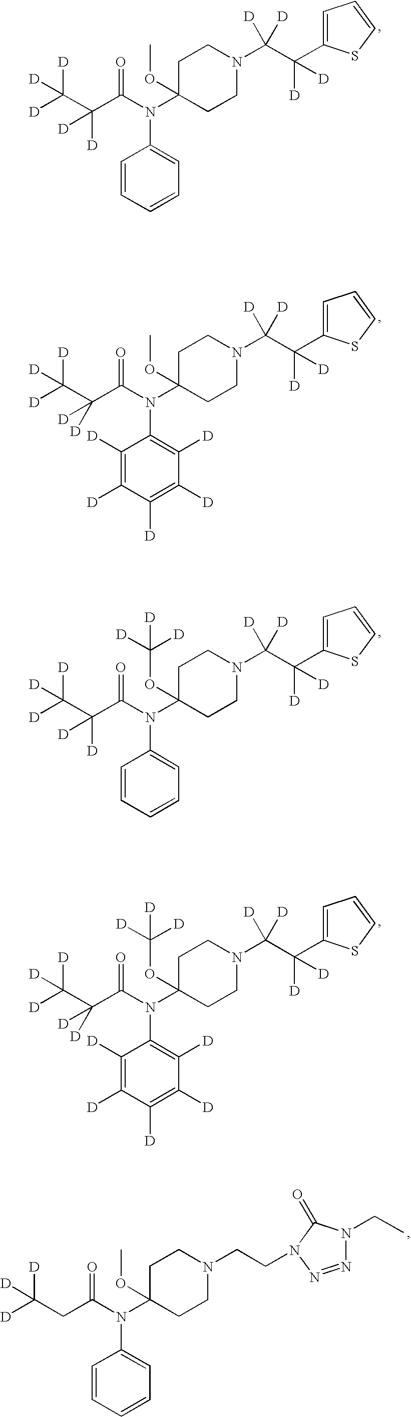

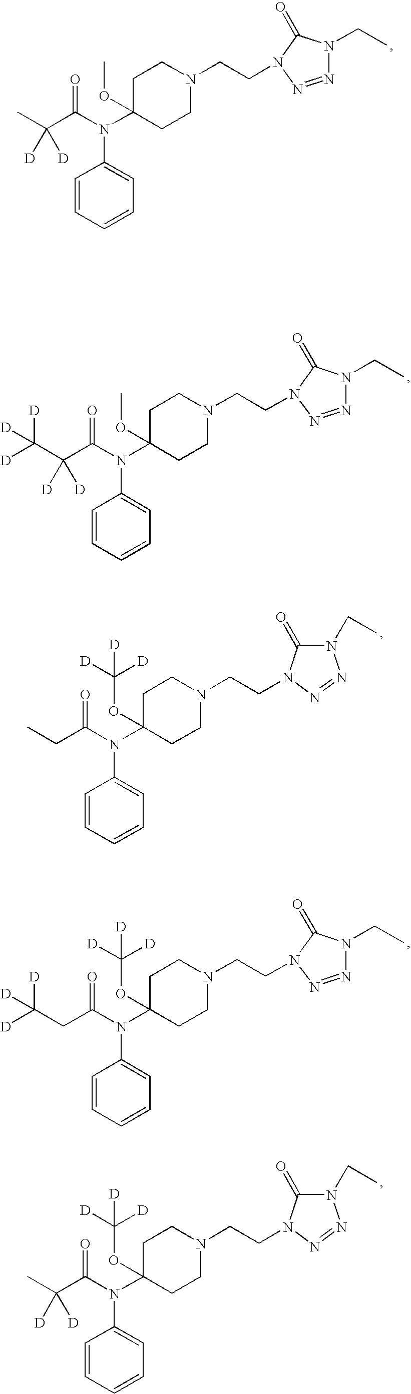

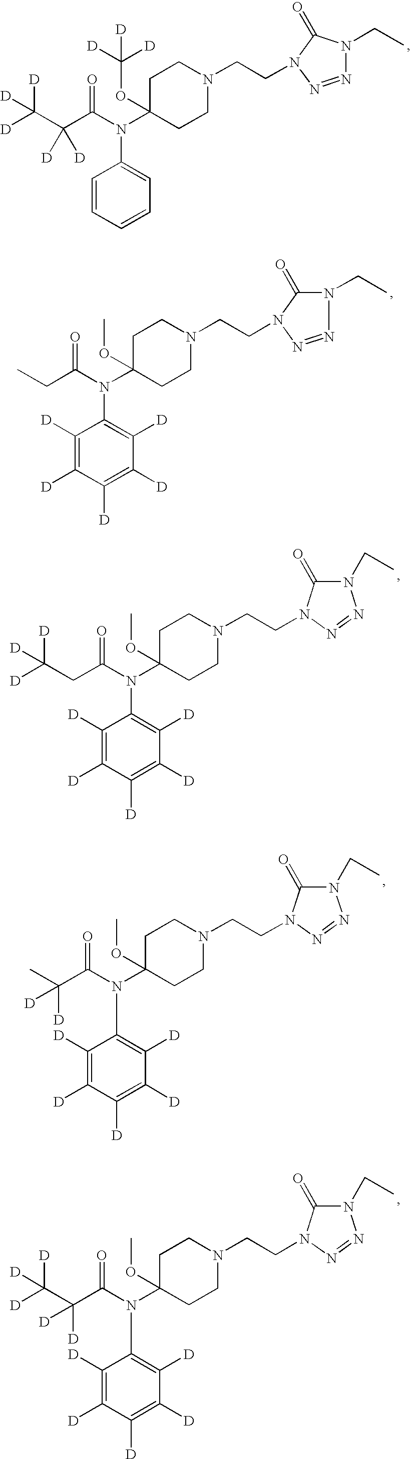

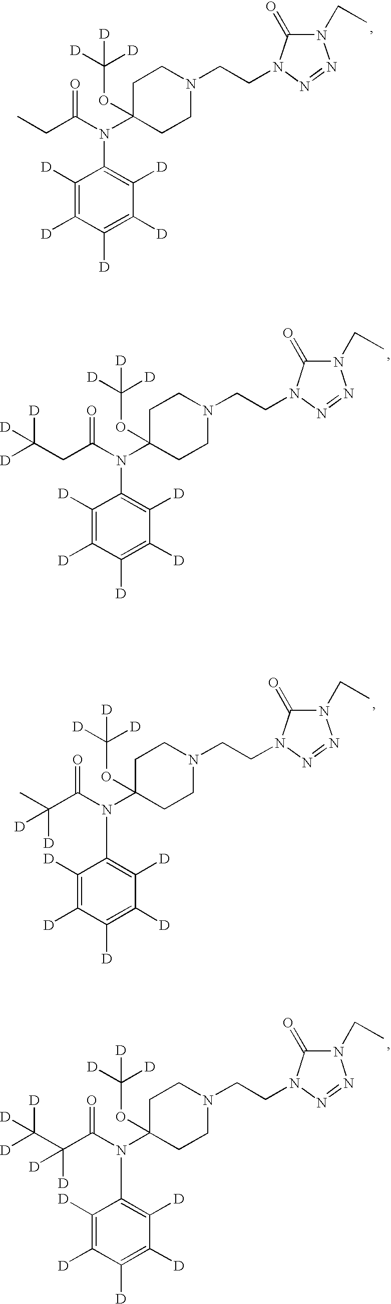

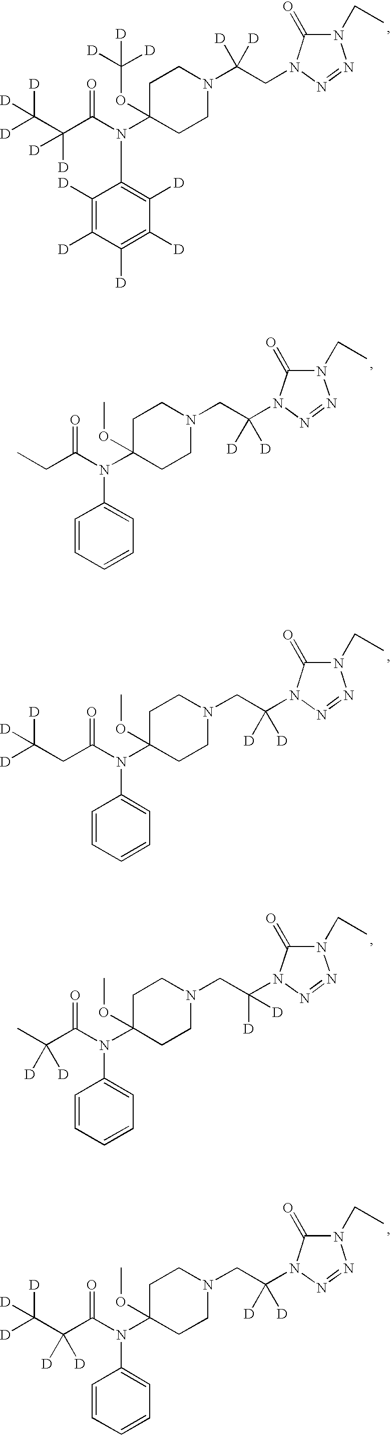

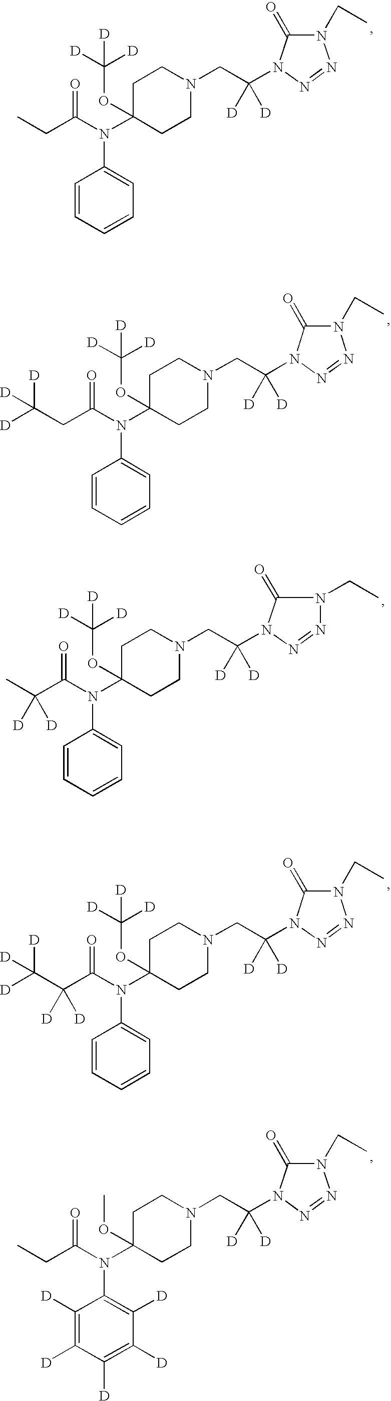

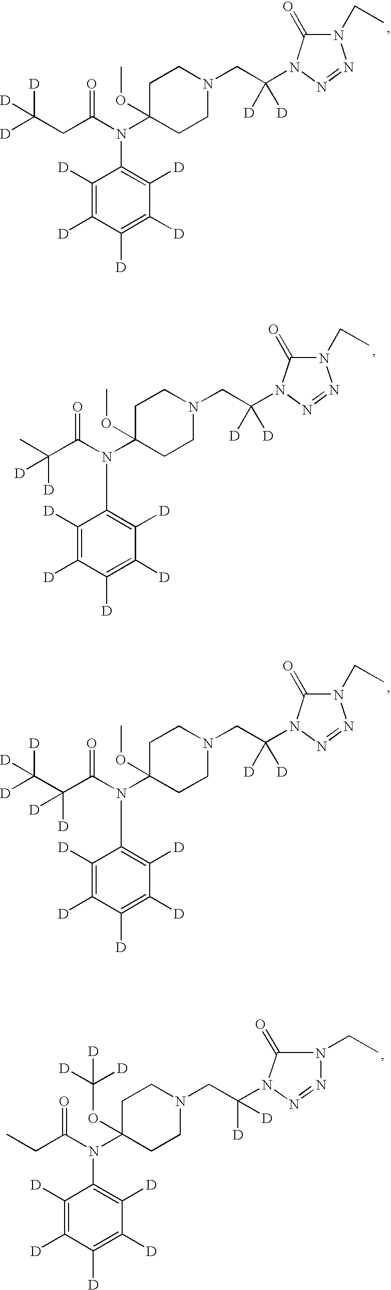

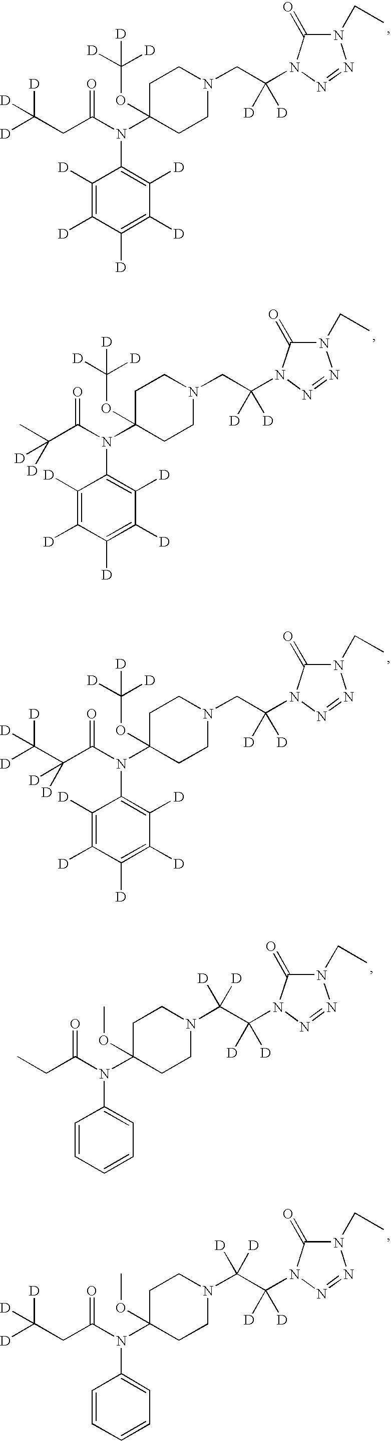

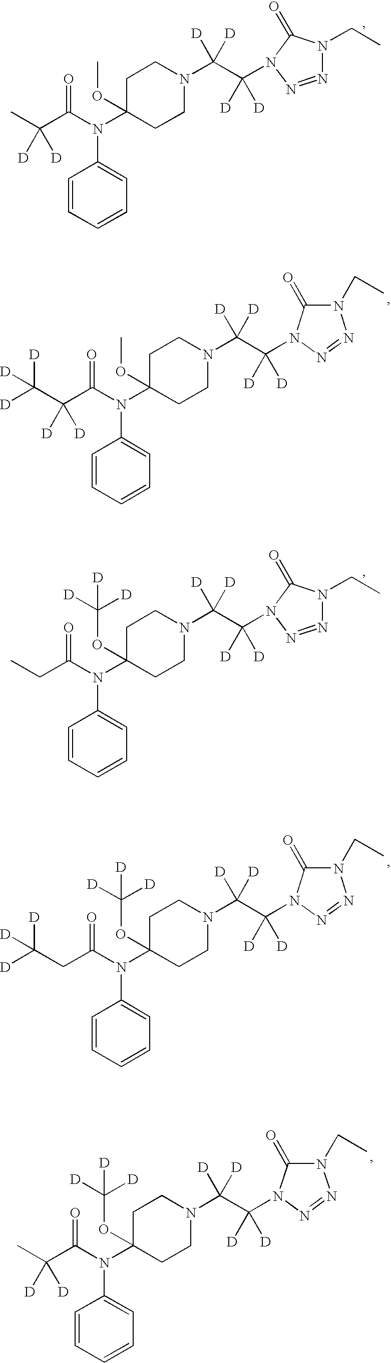

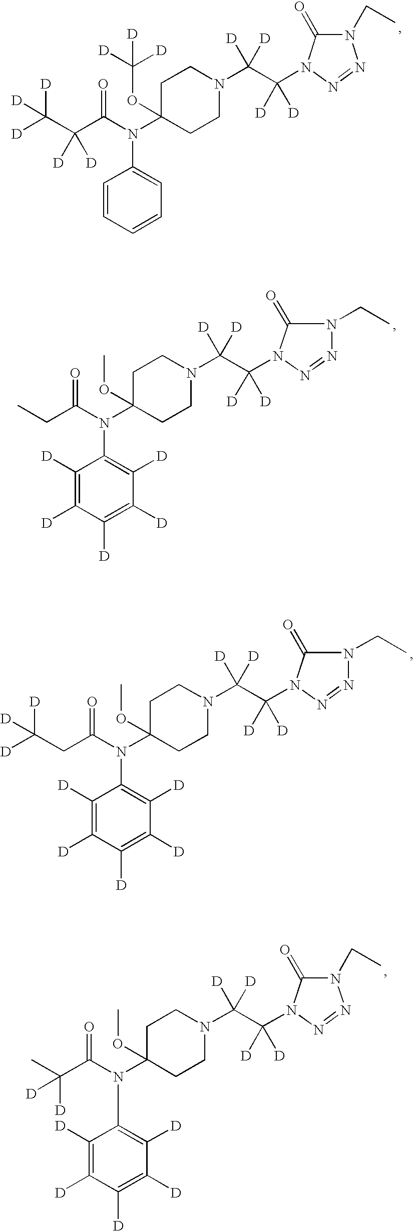

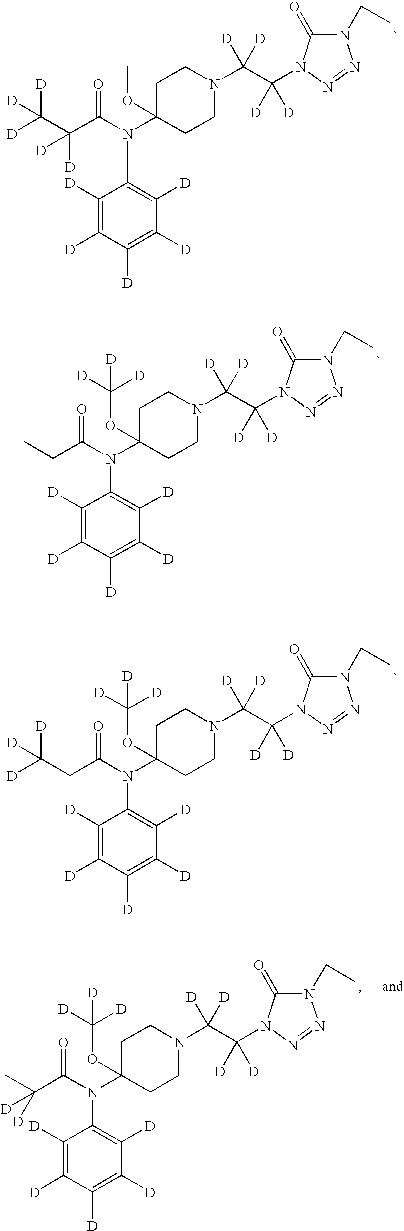

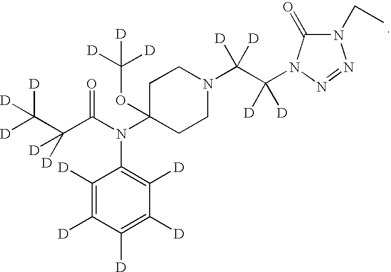

or a pharmaceutically acceptable salt, solvate, or prodrug thereof.

Changes in the metabolic properties of the compounds disclosed herein as compared to their non-isotopically enriched analogs can be shown using the following assays. Compounds listed above which have not yet been made and/or tested are predicted to have changed metabolic properties as shown by one or more of these assays as well.

### Biological Activity Assays

### In Vitro Liver Microsomal Stability Assay

Liver microsomal stability assays were conducted at 1 mg per mL liver microsome protein with an NADPH-generating system in 2% NaHCO₃ (2.2 mM NADPH, 25.6 mM glucose 6-phosphate, 6 units per mL glucose 6-phosphate dehydrogenase and 3.3 mM MgCl₂). Test compounds were prepared as solutions in 20% acetonitrile-water and added to the assay mixture (final assay concentration 1 μM) and incubated at 37° C. Final concentration of acetonitrile in the assay should be <1%. Aliquots (50 μL) were taken out at times 0, 0.25, 0.30, and 1 hours, and diluted with ice cold acetonitrile (200 μL) to stop the reactions. Samples were centrifuged at 12,000 RPM for 10 min to precipitate proteins. Supernatants were transferred to micro centrifuge tubes and stored for LC/MS/MS analysis of the degradation half-life of the test compounds. It has thus been found that compounds as disclosed in Examples 2, 3, 5, 6, 8, and 9 which have been tested in this assay showed improved degradation half-life, as compared to the non-isotopically enriched compounds, examples 1, 4, and 7. In certain embodiments, some of the compounds tested showed at least a 5%, 10%, or 13% increase in degradation half-life, as compared to the non-isotopically enriched drug.

### In Vitro Metabolism Using Human Cytochrome P₄₅₀ Enzymes

The cytochrome P₄₅₀ enzymes are expressed from the corresponding human cDNA using a baculovirus expression system (BD Biosciences, San Jose, Calif.). A 0.25 milliliter reaction mixture containing 0.8 milligrams per milliliter protein, 1.3 millimolar NADP⁺, 3.3 millimolar glucose-6-phosphate, 0.4 U/mL glucose-6-phosphate dehydrogenase, 3.3 millimolar magnesium chloride and 0.2 millimolar of a compound of Formula I, the corresponding non-isotopically enriched compound or standard or control in 100 millimolar potassium phosphate (pH 7.4) is incubated at 37° C. for 20 min. After incubation, the reaction is stopped by the addition of an appropriate solvent (e.g., acetonitrile, 20% trichloroacetic acid, 94% acetonitrile/6% glacial acetic acid, 70% perchloric acid, 94% acetonitrile/6% glacial acetic acid) and centrifuged (10,000 g) for 3 min. The supernatant is analyzed by HPLC/MS/MS.

| Cytochrome P₄₅₀ | Standard |
|---|---|
| CYP1A2 | Phenacetin |
| CYP2A6 | Coumarin |
| CYP2B6 | [¹³C]—(S)-mephenytoin |
| CYP2C8 | Paclitaxel |
| CYP2C9 | Diclofenac |
| CYP2C19 | [¹³C]—(S)-mephenytoin |
| CYP2D6 | (+/−)-Bufuralol |
| CYP2E1 | Chlorzoxazone |
| CYP3A4 | Testosterone |
| CYP4A | [¹³C]-Lauric acid |

### Monoamine Oxidase A Inhibition and Oxidative Turnover

The procedure is carried out using the methods described by Weyler, Journal of Biological Chemistry 1985, 260, 13199-13207, which is hereby incorporated by reference in its entirety. Monoamine oxidase A activity is measured spectrophotometrically by monitoring the increase in absorbance at 314 nm on oxidation of kynuramine with formation of 4-hydroxyquinoline. The measurements are carried out, at 30° C., in 50 mM NaPᵢ buffer, pH 7.2, containing 0.2% Triton X-100 (monoamine oxidase assay buffer), plus 1 mM kynuramine, and the desired amount of enzyme in 1 mL total volume.

### Monooamine Oxidase B Inhibition and Oxidative Turnover

The procedure is carried out as described in Uebelhack et al., Pharmacopsychiatry 1998, 31(5), 187-192, which is hereby incorporated by reference in its entirety.

Identification of fentanyl, alfentanil, sufentanil and Their Major Metabolites in Human Urine by Liquid Chromatography/Tandem Mass Spectrometry

The procedure is carried out as described in Geyer et al., European Journal of Mass Spectrometry 2005, 11(4), 419-427, which is hereby incorporated by reference in its entirety.

Determination of fentanyl in Human Plasma and fentanyl and norfentanyl in Human Urine Using LC-MS/MS

The procedure is carried out as described in Huynh et al., Journal of Pharmaceutical and Biomedical Analysis 2005, 37(5), 1095-1100, which is hereby incorporated by reference in its entirety.

Highly Sensitive Gas Chromatographic-Mass Spectrometric Screening Method for the Determination of Picogram Levels of fentanyl, sufentanil and alfentanil and Their Major Metabolites in Urine

The procedure is carried out as described in Van Nimmen et al., Journal of Chromatography, B: Analytical Technologies in the Biomedical and Life Sciences 2004, 804(2), 375-387, which is hereby incorporated by reference in its entirety.

Analysis of fentanyl and sufentanil in Hair by GC/MS/MS

The procedure is carried out as described in Sachs et al., International Journal of Legal Medicine 1996, 109(4), 213-5, which is hereby incorporated by reference in its entirety.

Determination of alfentanil and noralfentanil in Human Plasma by Gas Chromatography-Mass Spectrometry

The procedure is carried out as described in Mautz et al., Journal of chromatography. B, Biomedical applications 1994, 658(1), 149-53, which is hereby incorporated by reference in its entirety.

Transcriptional Regulation of μ-Opioid Receptor Gene by cAMP Pathway

The procedure is carried out as described in Lee, et al., Molecular Pharmacology 2003, 64(6), 1410-1418, which is hereby incorporated by reference in its entirety.

### Binding Affinity to and Dependence on Some Opioids in Sf9 Insect Cells Expressing Human μ-Opioid Receptor

The procedure is carried out as described in Liu, et al., Acta Pharmacologica Sinica 2003, 24(9), 859-863, which is hereby incorporated by reference in its entirety.

### Characterization of Opioid Agonist Efficacy in a C6 Glioma Cell Line Expressing the μ-Opioid Receptor

The procedure is carried out as described in Emmerson, et al., Journal of Pharmacology and Experimental Therapeutics 1996, 278(3), 1121-1127, which is hereby incorporated by reference in its entirety.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A compound having structural Formula I 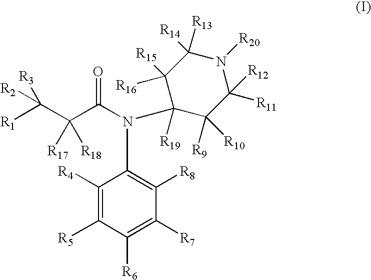
or a pharmaceutically acceptable salt thereof, wherein:
R₁-R₁₈, R₂₁-R₂₈, R₃₀-R₅₀ are independently selected from the group consisting of hydrogen and deuterium;
R₁₉ is selected from the group consisting of hydrogen, deuterium, and 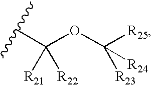
R₂₀ is selected from the group consisting of consisting of hydrogen, deuterium, 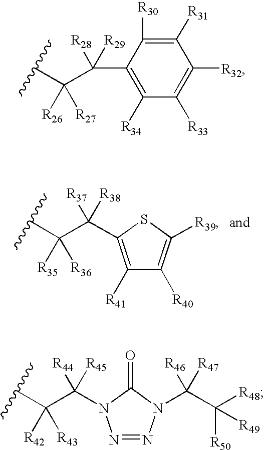
R₂₉ is selected from the group consisting of consisting of hydrogen, deuterium, hydroxyl, and —O-D; and
at least one of R₁-R₅₀ is deuterium; and
with the proviso that the compound cannot be selected from the group consisting of:

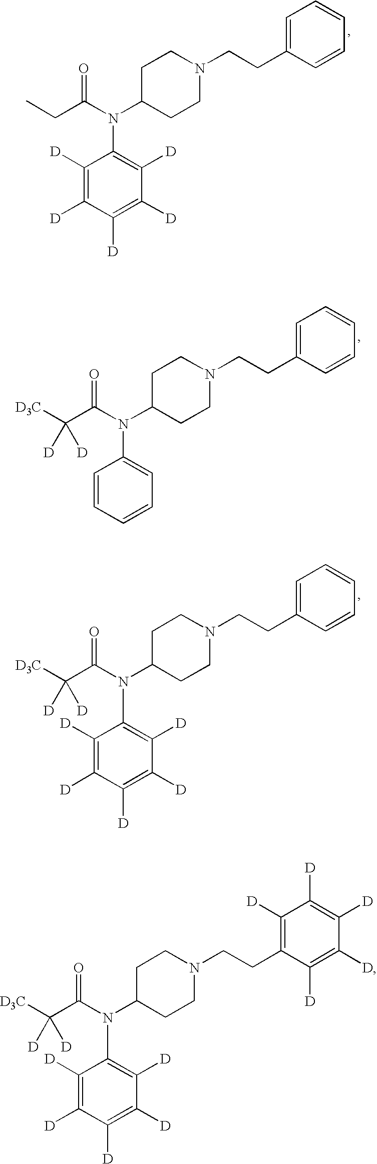

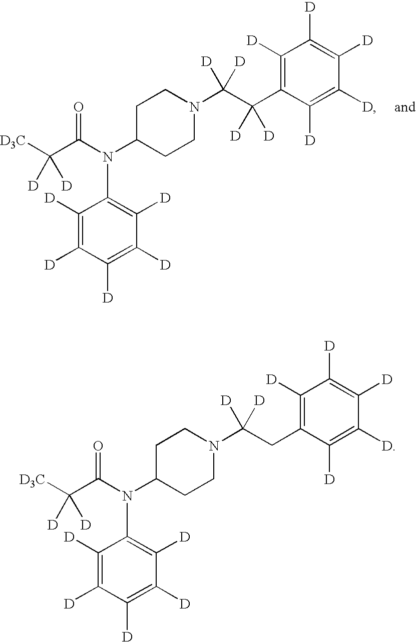

2. The compound as recited in claim 1 wherein at least one of R₁-R₅₀ independently has deuterium enrichment of no less than about 10%.

3. The compound as recited in claim 1 wherein at least one of R₁-R₅₀ independently has deuterium enrichment of no less than about 50%.

4. The compound as recited in claim 1 wherein at least one of R₁-R₅₀ independently has deuterium enrichment of no less than about 90%.

5. The compound as recited in claim 1 wherein at least one of R₁-R₅₀ independently has deuterium enrichment of no less than about 98%.

6. The compound as recited in claim 1 wherein said compound has a structural formula selected from the group consisting of 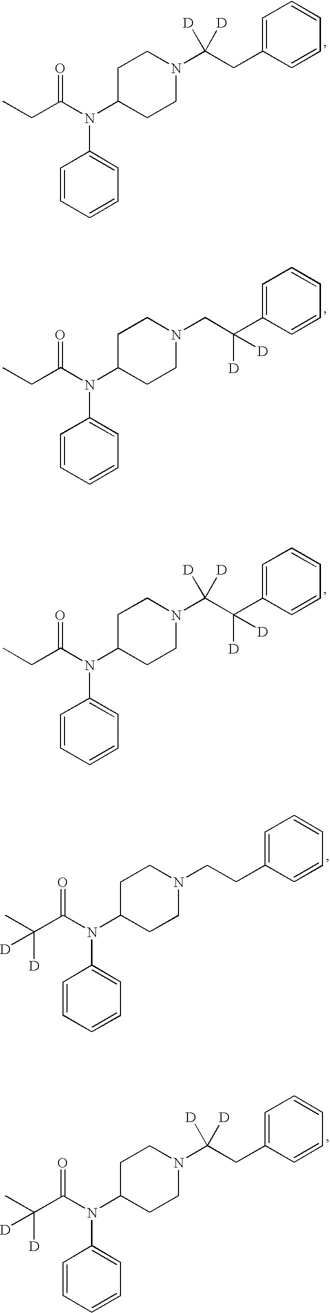 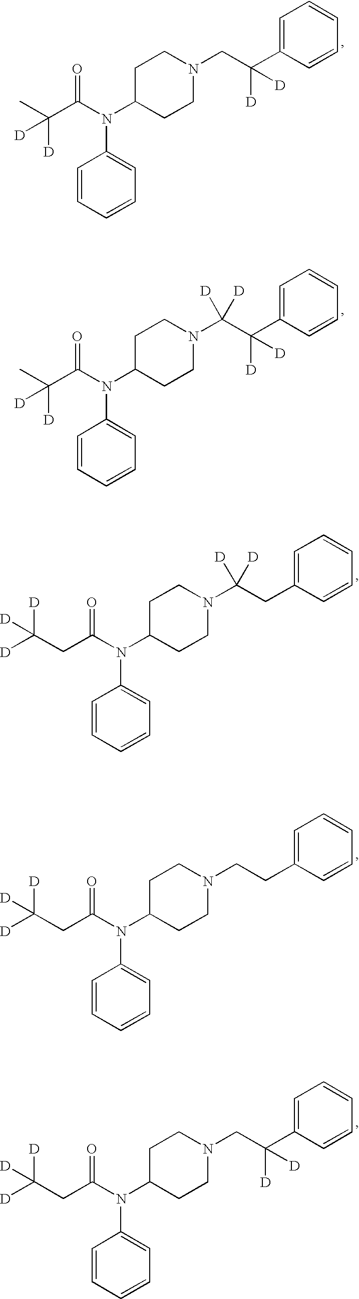 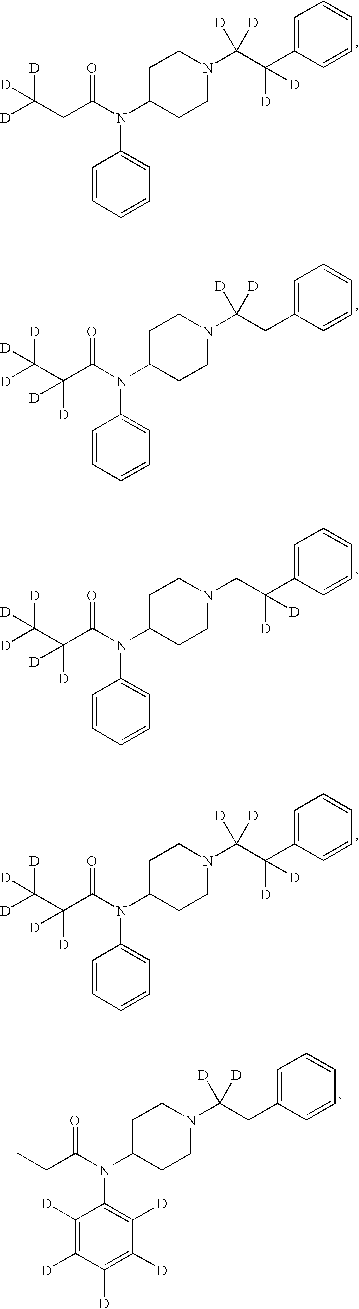 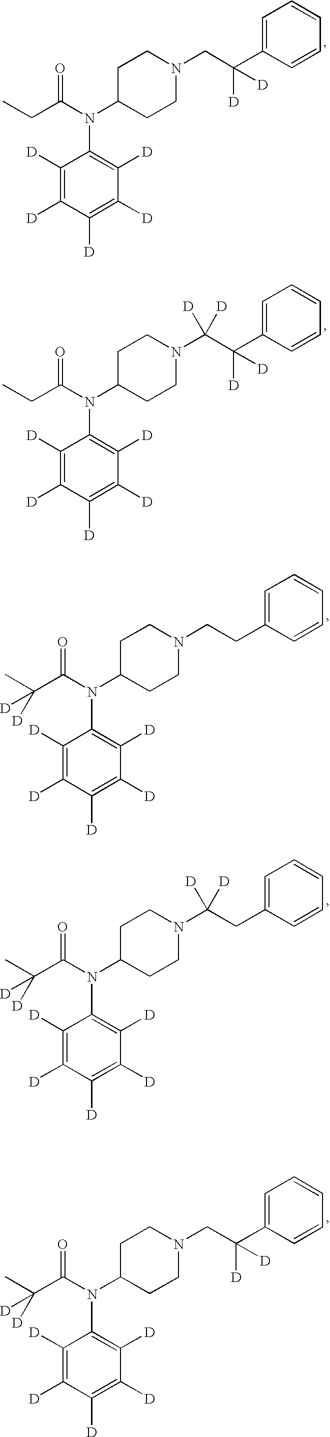 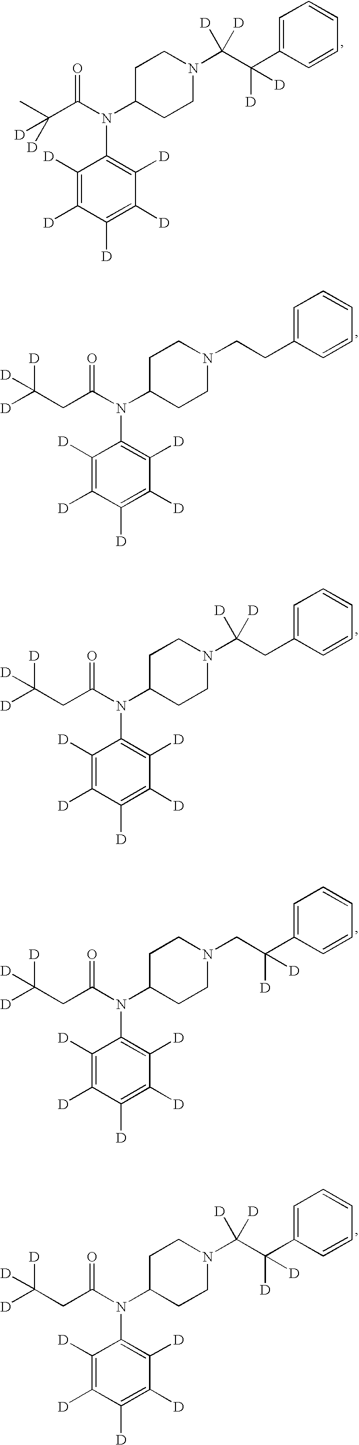 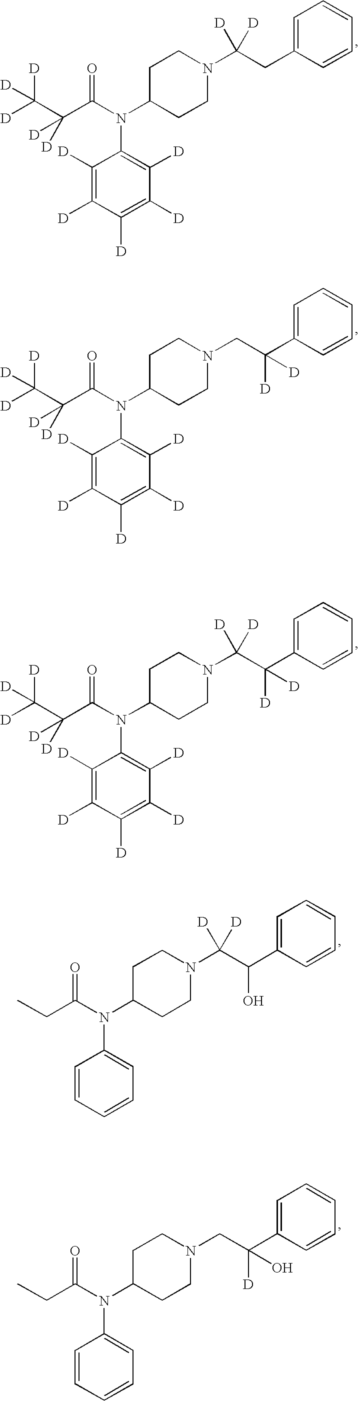 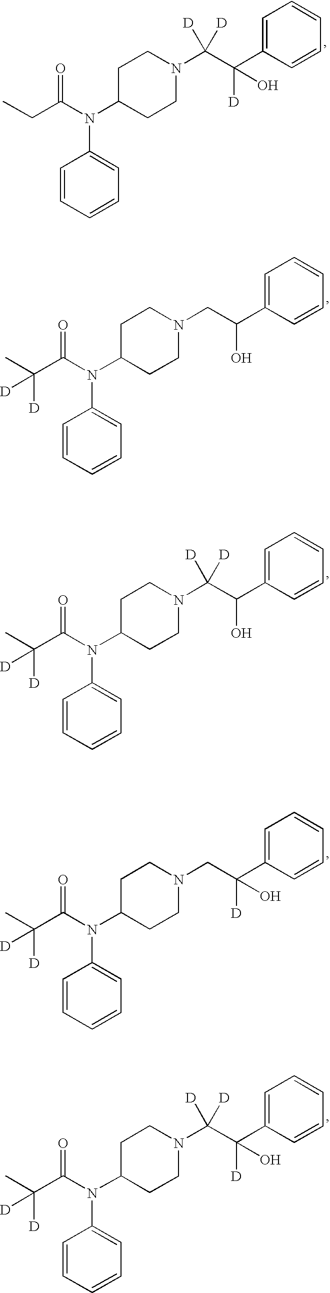 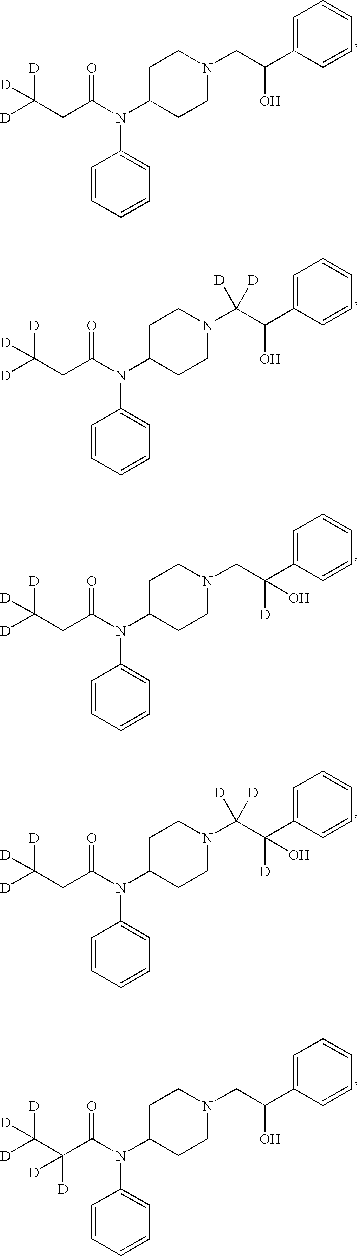 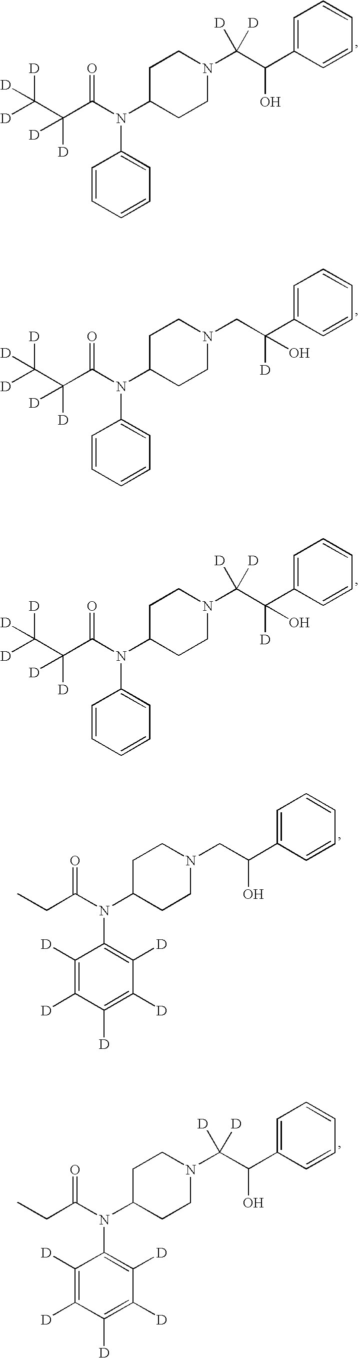 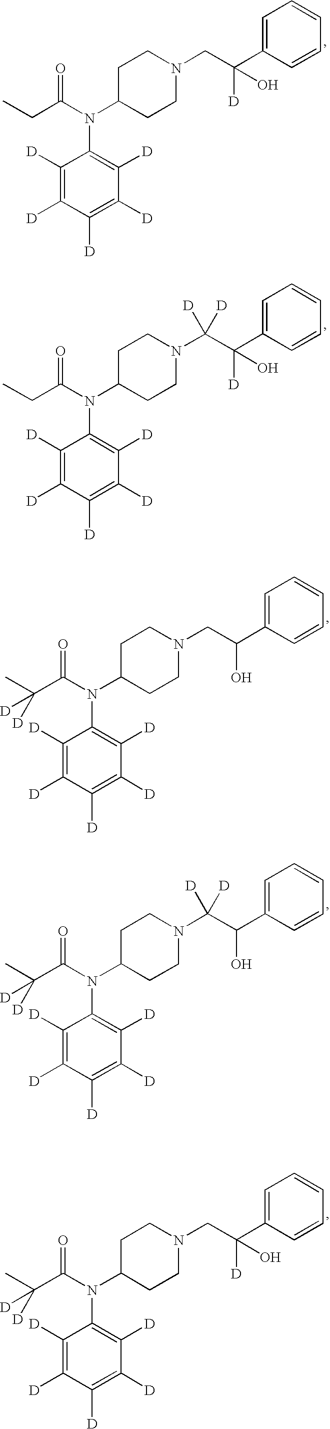 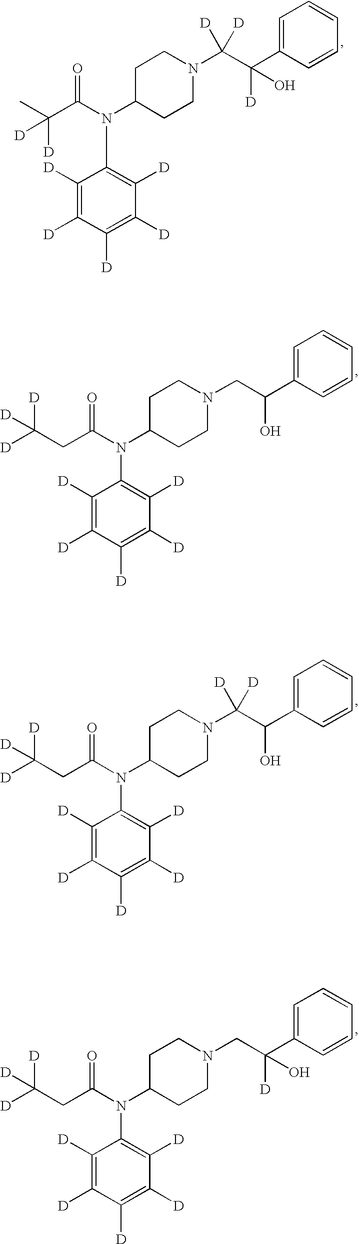 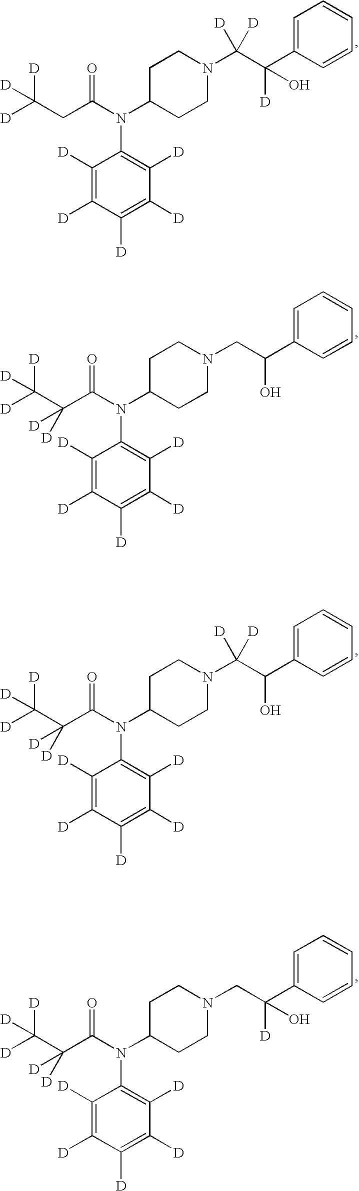 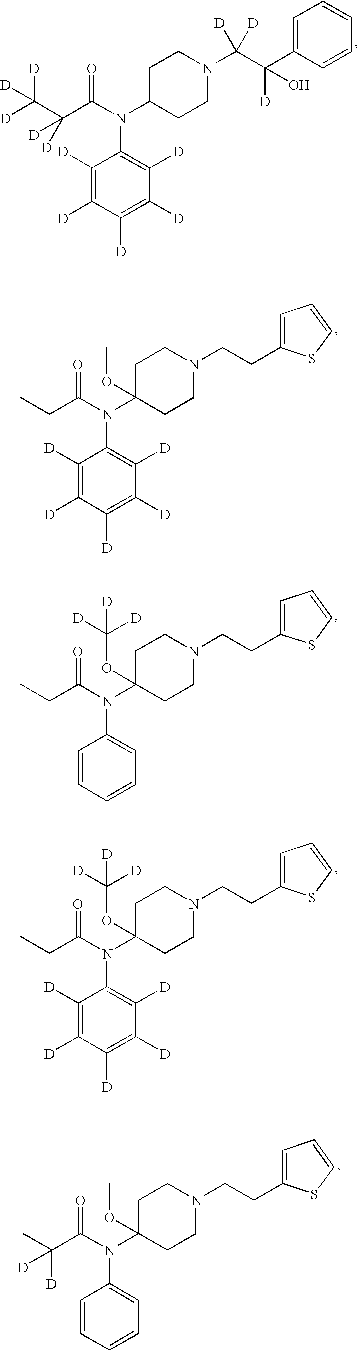 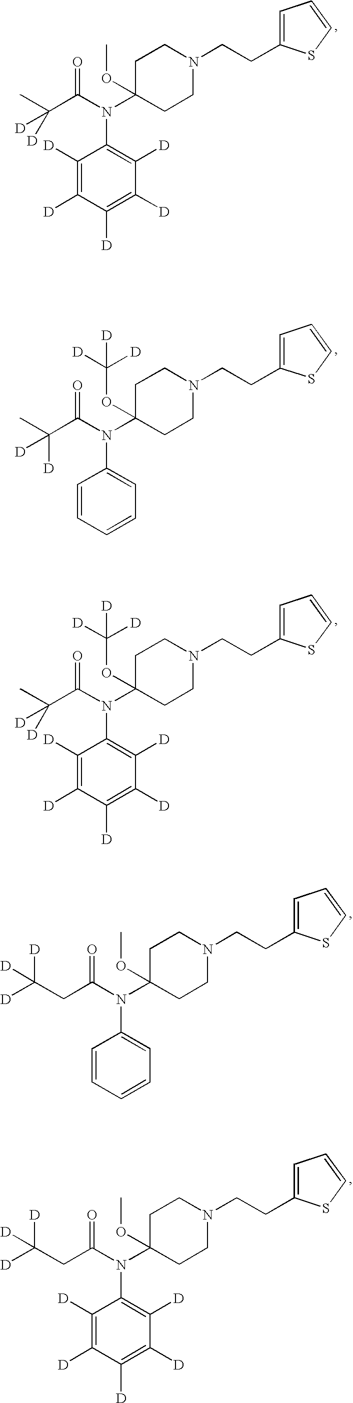 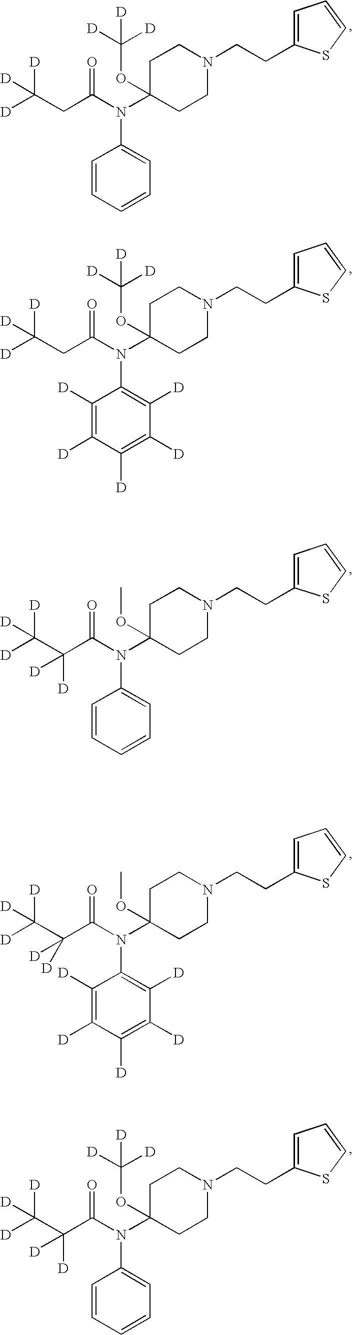 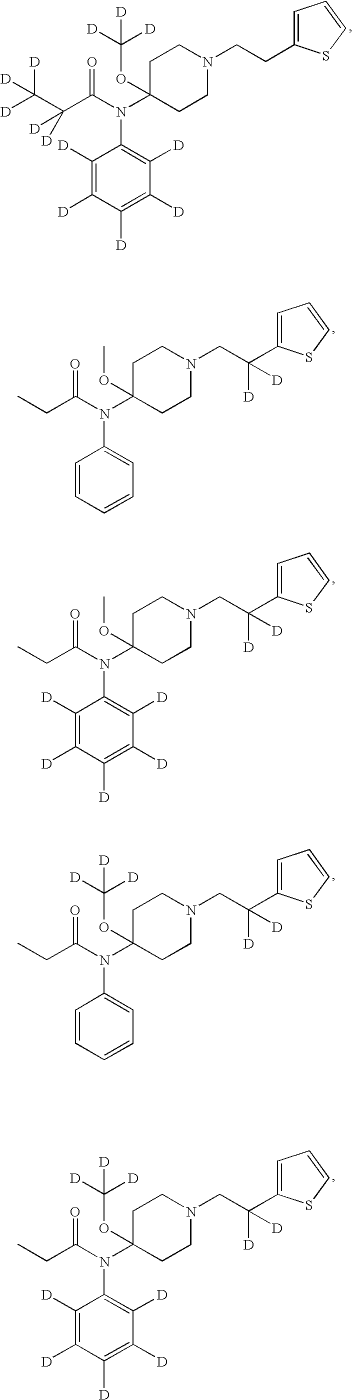 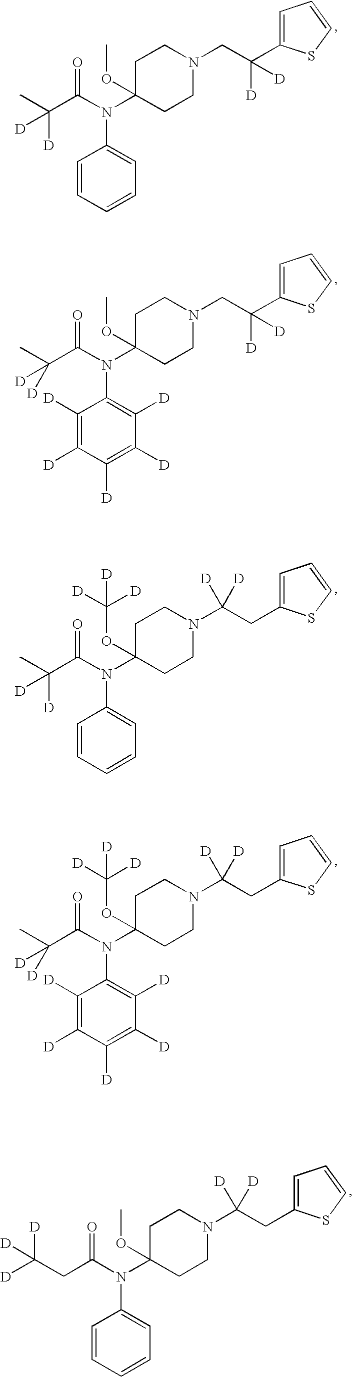 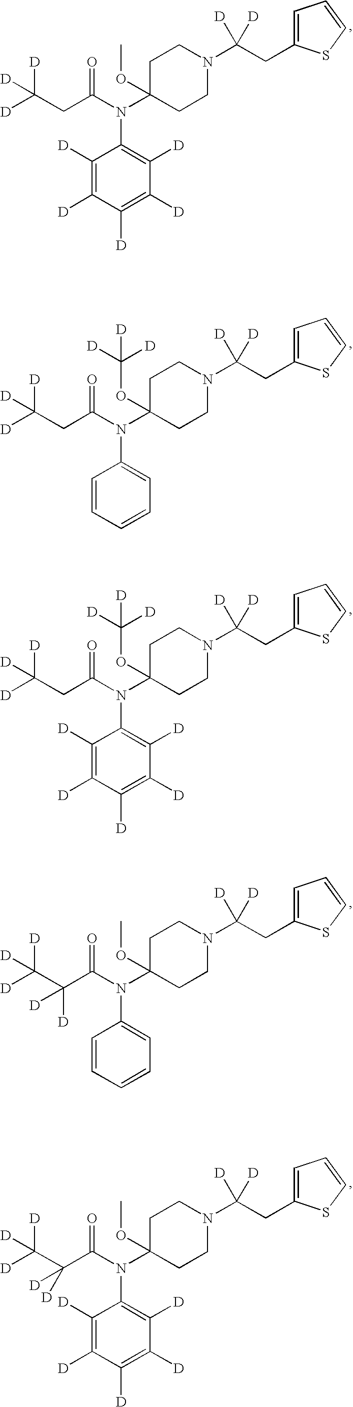 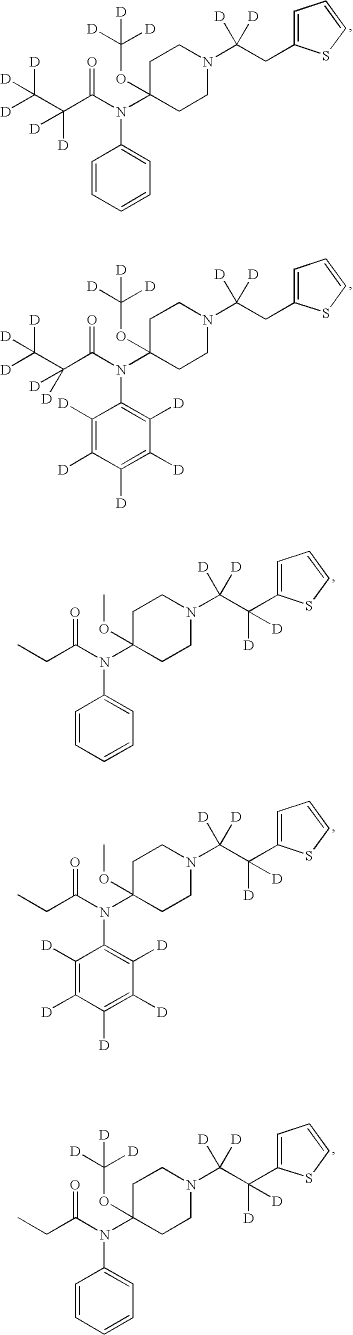 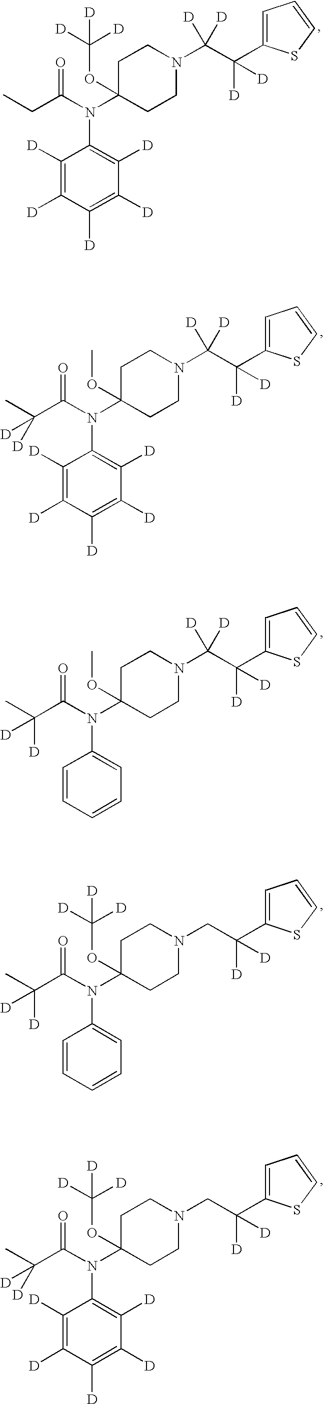 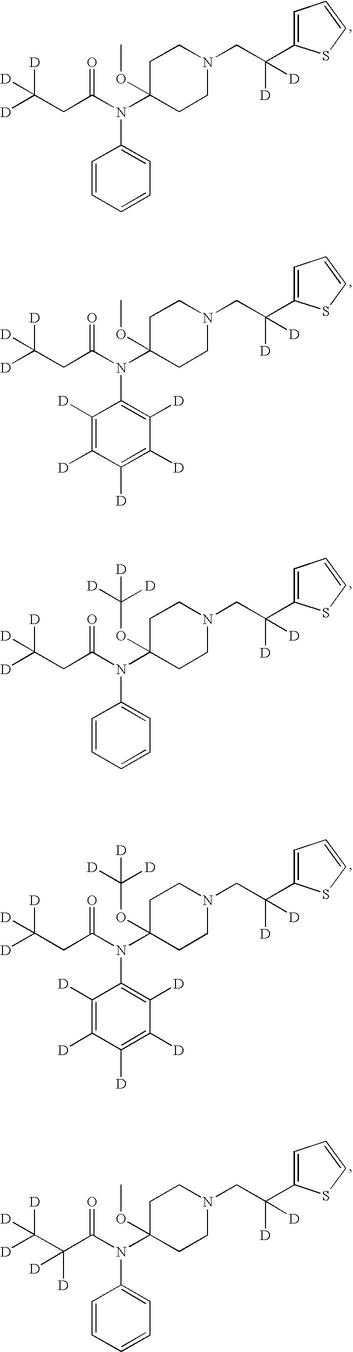 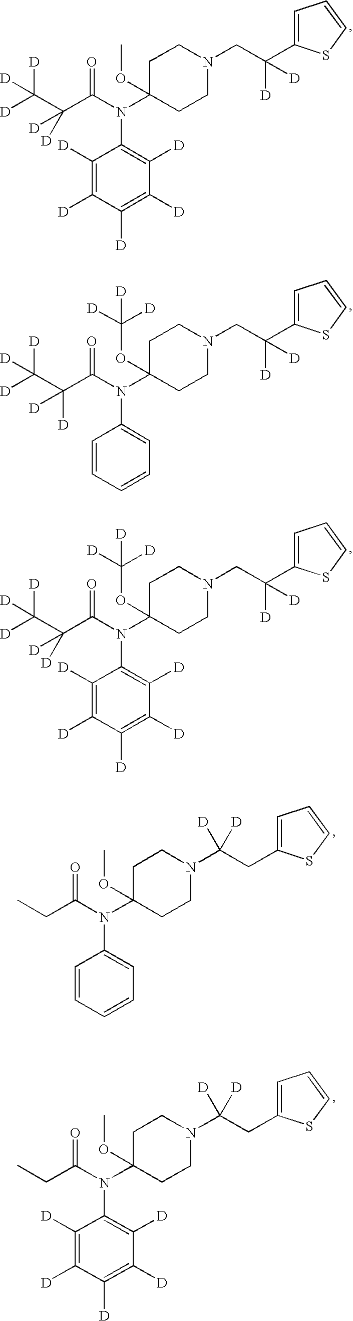 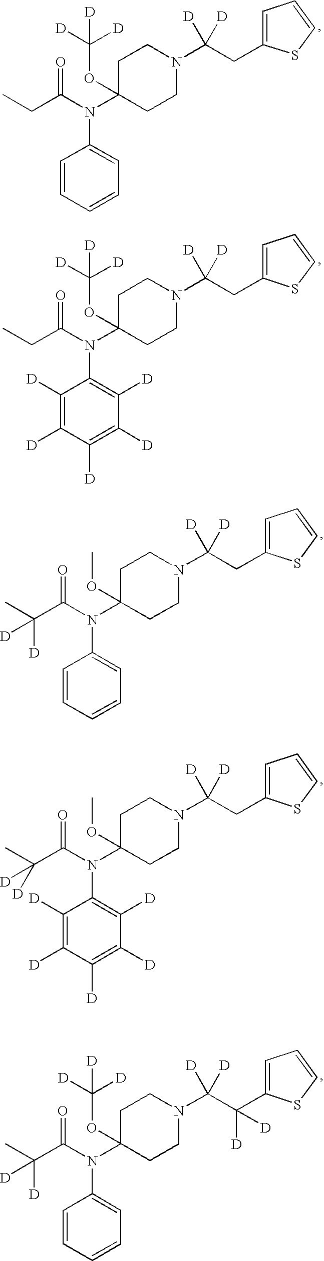 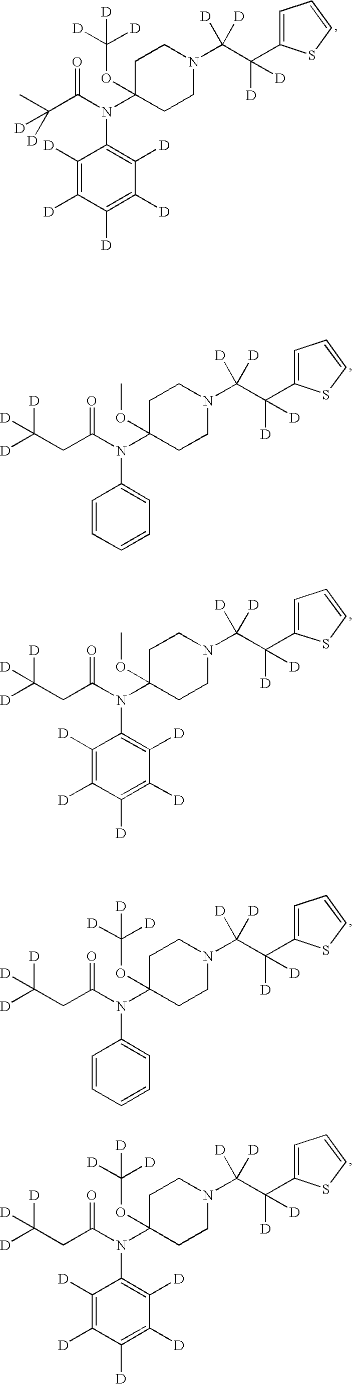 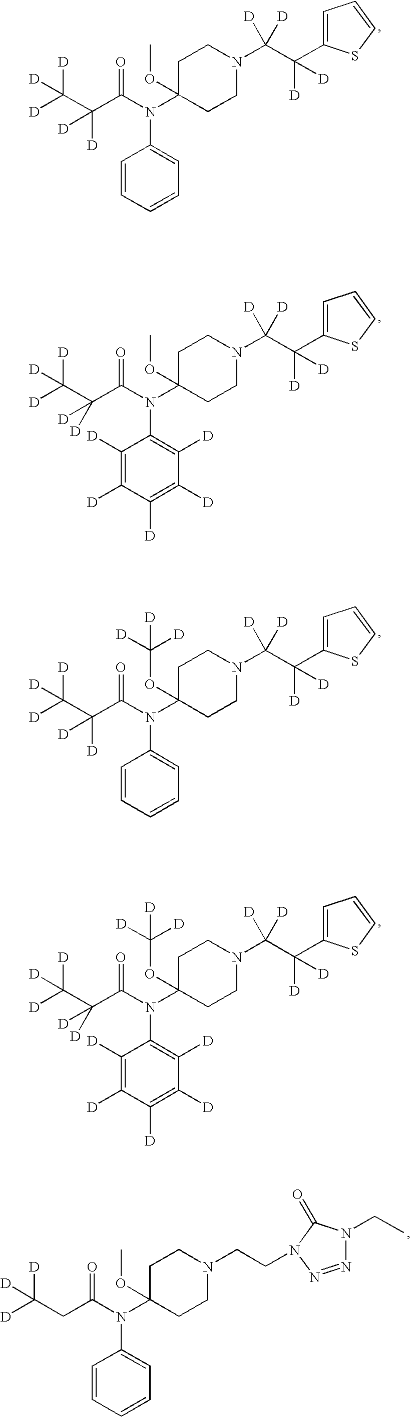 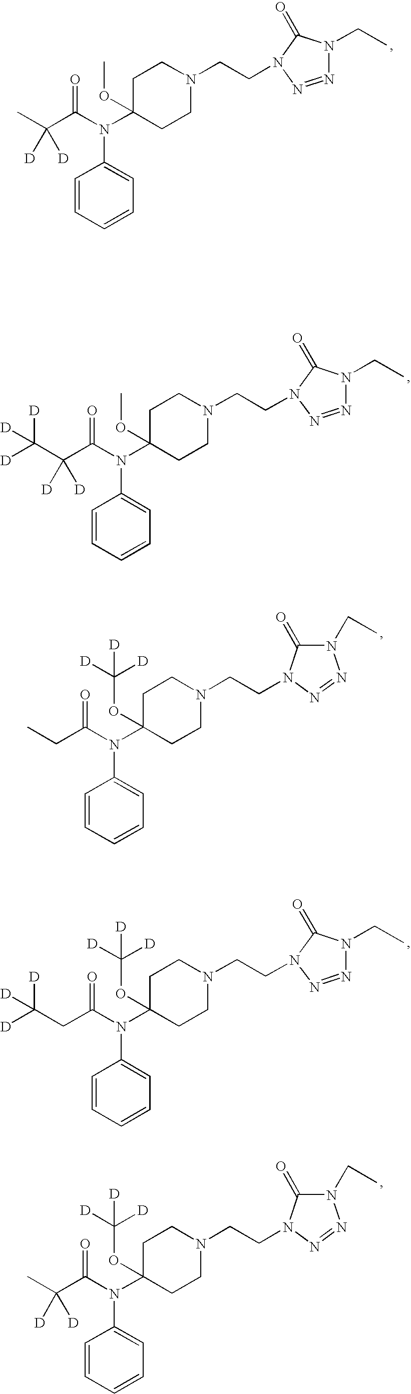 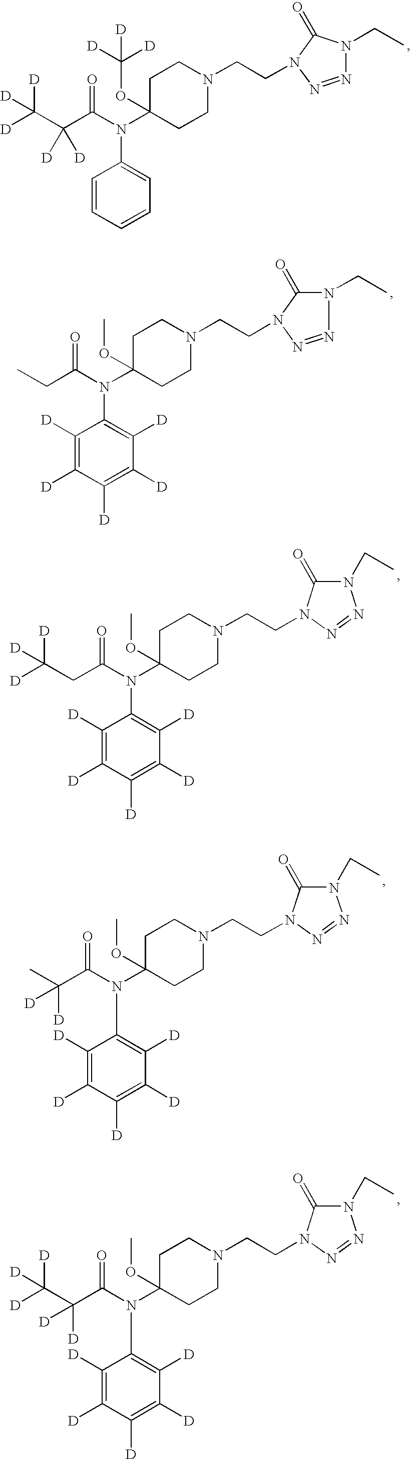 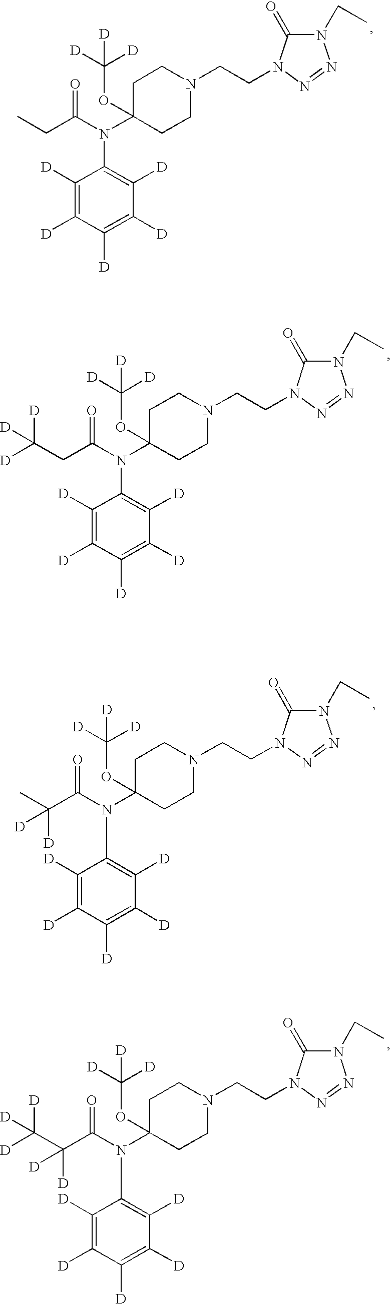 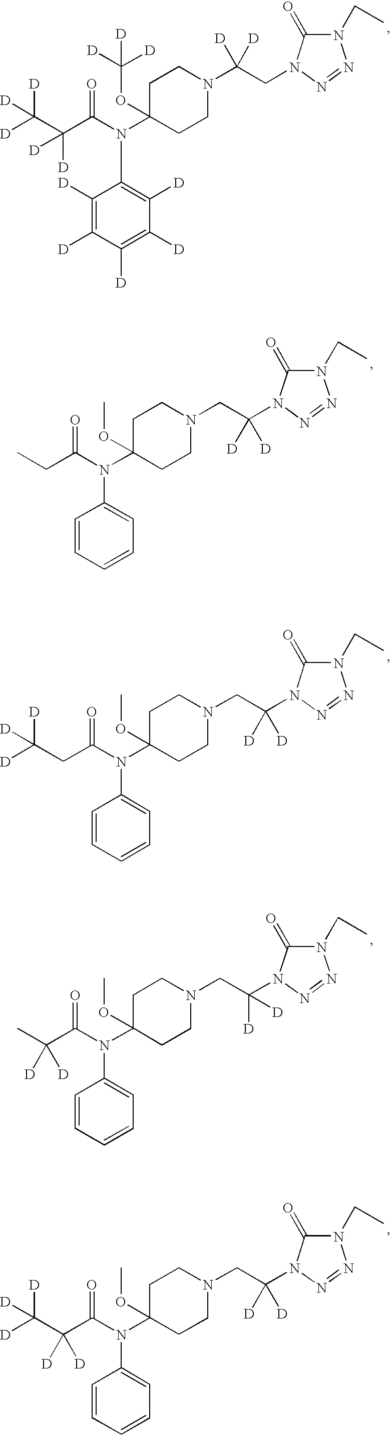 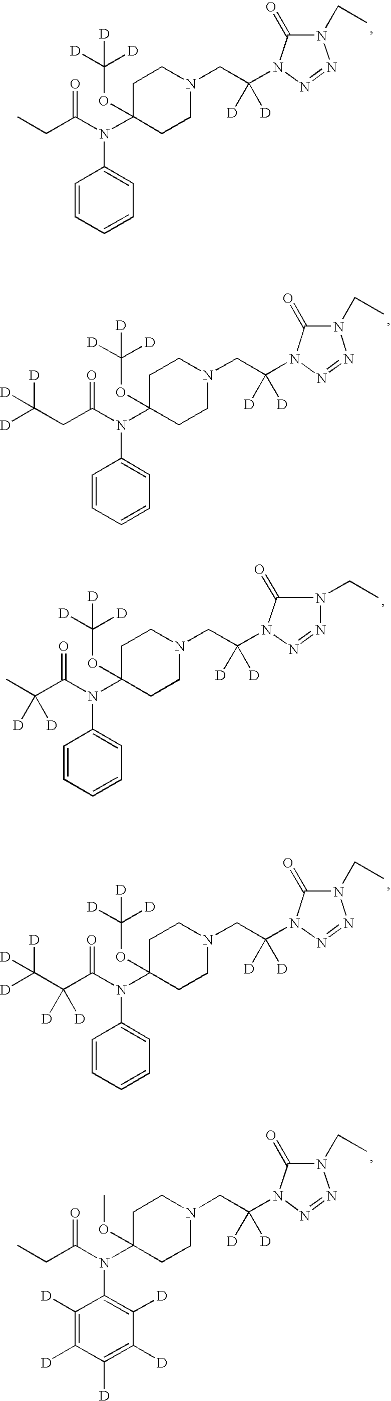 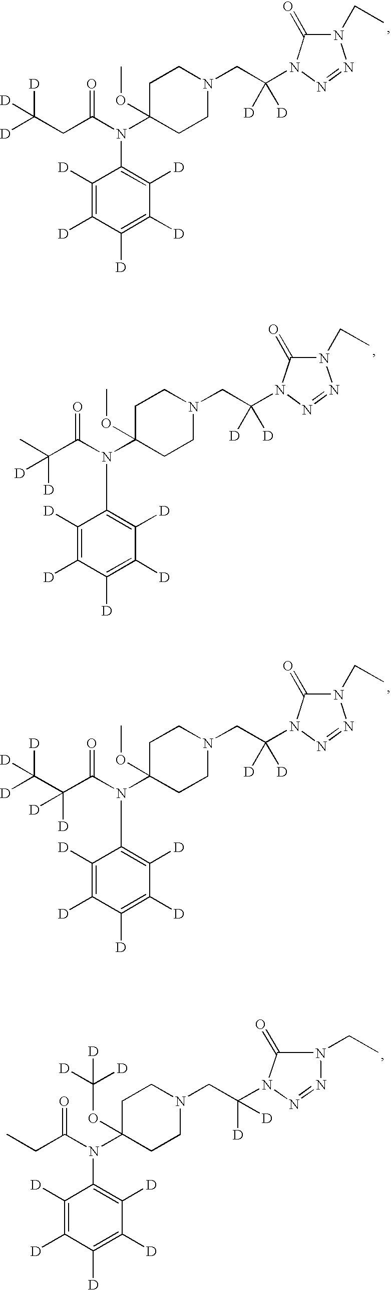 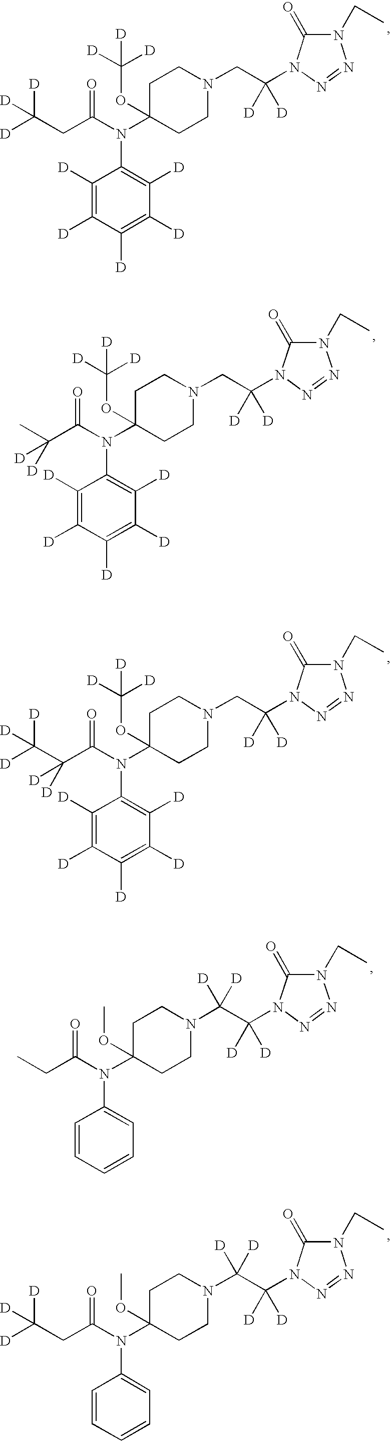 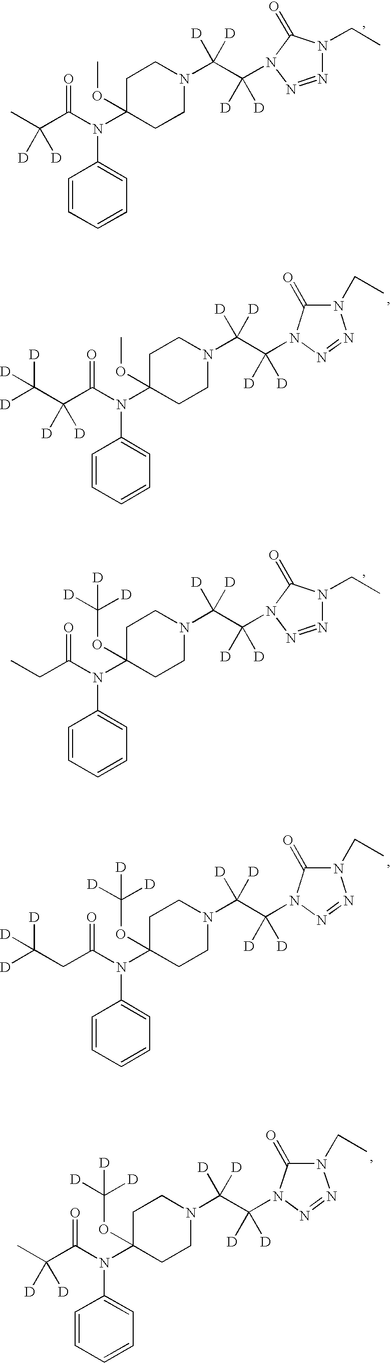 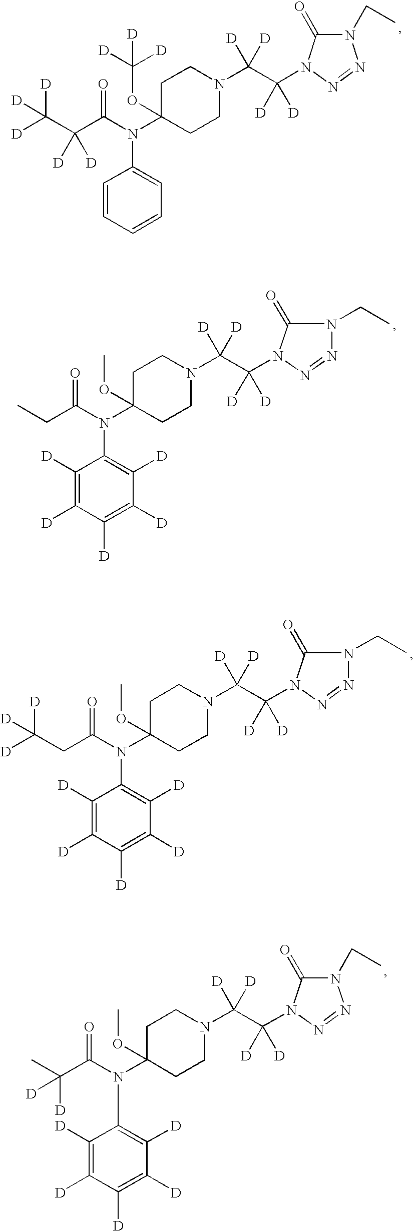 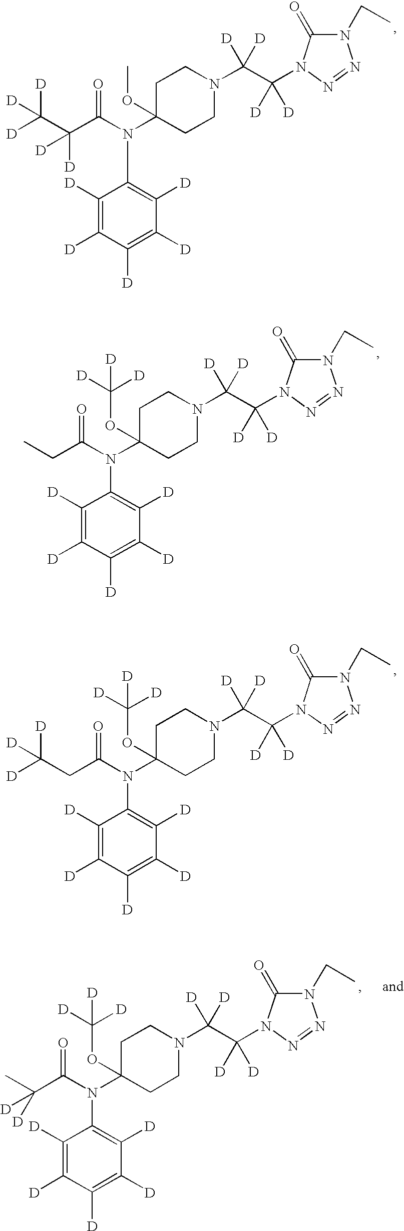 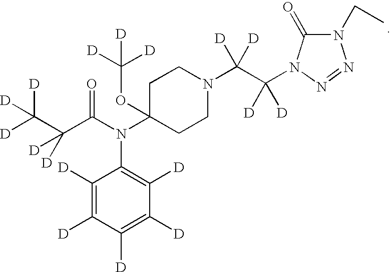
or a pharmaceutically acceptable salt thereof.

7. The compound as recited in claim 6 wherein each position represented as D has deuterium enrichment of no less than about 10%.

8. The compound as recited in claim 6 wherein each position represented as D has deuterium enrichment of no less than about 50%.

9. The compound as recited in claim 6 wherein each position represented as D has deuterium enrichment of no less than about 90%.

10. The compound as recited in claim 6 wherein each position represented as D has deuterium enrichment of no less than about 98%.

11. A pharmaceutical composition comprising a compound as recited in claim 1 together with a pharmaceutically acceptable carrier.

12. The compound as recited in claim 1 wherein said compound has a structural formula selected from the group consisting of

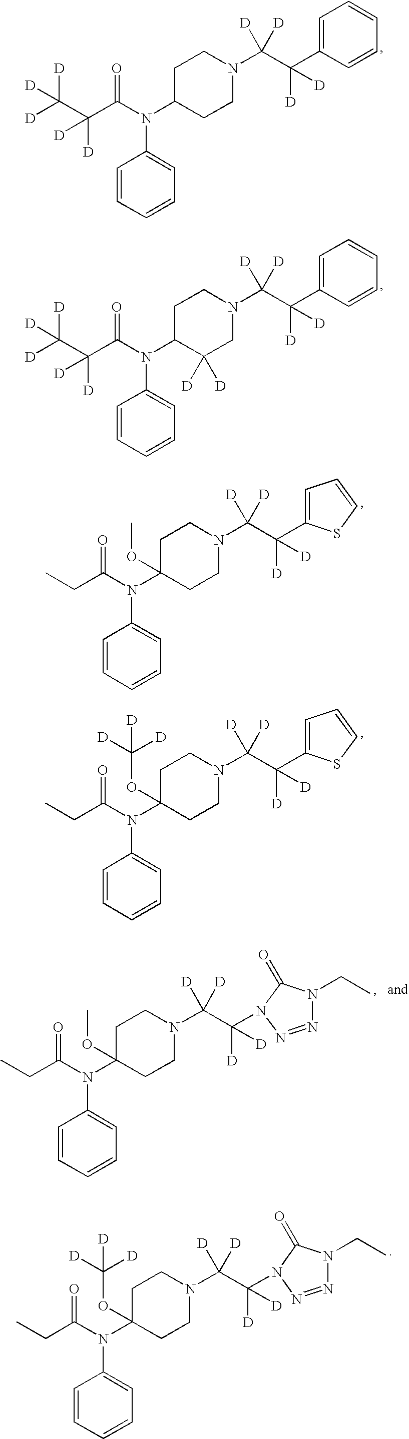

13. The compound as recited in claim 12 wherein each position represented as D has deuterium enrichment of no less than about 10%.

14. The compound as recited in claim 12 wherein each position represented as D has deuterium enrichment of no less than about 50%.

15. The compound as recited in claim 12 wherein each position represented as D has deuterium enrichment of no less than about 90%.

16. The compound as recited in claim 12 wherein each position represented as D has deuterium enrichment of no less than about 98%.

17. A method of treatment of an opioid receptor-mediated disorder comprising the administration of a therapeutically effective amount of a compound having structural Formula I: 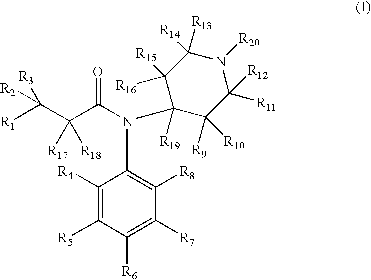
or a pharmaceutically acceptable salt thereof, wherein:
R₁-R₁₈, R₂₁-R₂₈, R₃₀-R₅₀ are independently selected from the group consisting of hydrogen and deuterium;
R₁₉ is selected from the group consisting of hydrogen, deuterium, and 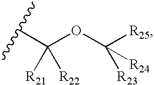
R₂₀ is selected from the group consisting of consisting of hydrogen, deuterium, 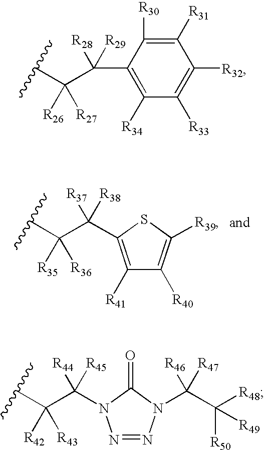
R₂₉ is selected from the group consisting of consisting of hydrogen, deuterium, hydroxyl, and —O-D; and
at least one of R₁-R₅₀ is deuterium.

18. The method as recited in claim 17 wherein said opioid receptor-mediated disorder is selected from the group consisting of preoperative pain, intraoperative pain, postoperative pain, partuition, oncological pain, neuropathy, osteoarthritis, rheumatoid arthritis, fibromyalgia, pancreatitis, herniated discs, migraine, trigeminal neuralgia, sciatica, myocardial infarctions, any procedure which requires anesthesia or sedation, and any disorder associated with moderate to severe acute or chronic pain.

19. The method as recited in claim 17 further comprising the administration of an additional therapeutic agent.

20. The method as recited in claim 19 wherein said additional therapeutic agent is selected from the group consisting of opioids, steroidal drugs, local or general anesthetics, sepsis treatments, antibacterial agents, antifungal agents, anticoagulants, thrombolytics, non-steroidal anti-inflammatory agents, antiplatelet agents, NRIs, DARIs, SNRIs, sedatives, NDRIs, SNDRIs, monoamine oxidase inhibitors, hypothalamic phospholipids, ECE inhibitors, thromboxane receptor antagonists, potassium channel openers, thrombin inhibitors, hypothalamic phospholipids, growth factor inhibitors, anti-platelet agents, P2Y(AC) antagonists, anticoagulants, low molecular weight heparins, Factor VIIa Inhibitors and Factor Xa Inhibitors, renin inhibitors, NEP inhibitors, vasopepsidase inhibitors, HMG CoA reductase inhibitors, squalene synthetase inhibitors, fibrates, bile acid sequestrants, anti-atherosclerotic agents, MTP Inhibitors, calcium channel blockers, potassium channel activators, alpha-muscarinic agents, beta-muscarinic agents, antiarrhythmic agents, diuretics, anti-diabetic agents, mineralocorticoid receptor antagonists, growth hormone secretagogues, aP2 inhibitors, phosphodiesterase inhibitors, protein tyrosine kinase inhibitors, antiproliferatives, chemotherapeutic agents, immunosuppressants, anticancer agents and cytotoxic agents, antimetabolites, antibiotics, farnesyl-protein transferase inhibitors, hormonal agents, microtubule-disruptor agents, microtubule-stabilizing agents, plant-derived products, epipodophyllotoxins, taxanes, topoisomerase inhibitors, prenyl-protein transferase inhibitors, cyclosporins, cytotoxic drugs, TNF-alpha inhibitors, anti-TNF antibodies and soluble TNF receptors, cyclooxygenase-2 (COX-2) inhibitors, and miscellaneous agents.

21. The method as recited in claim 20 wherein said opioid is selected from the group consisting of morphine, codeine, thebain, diacetylmorphine, oxycodone, hydrocodone, hydromorphone, oxymorphone, nicomorphine, fentanyl, α-methylfentanyl, alfentanil, sufentanil, remifentanyl, carfentanyl, ohmefentanyl, pethidine, ketobemidone, propoxyphene, dextropropoxyphene, methadone, loperamide, pentazocine, buprenorphine, etorphine, butorphanol, nalbufine, levorphanol, naloxone, naltrexone, and tramadol.

22. The method as recited in claim 20 wherein said steroidal drug is selected from the group consisting of aldosterone, beclometasone, betamethasone, deoxycorticosterone acetate, fludrocortisone acetate, hydrocortisone, prednisolone, prednisone, methylprenisolone, dexamethasone, and triamcinolone.

23. The method as recited in claim 20 wherein said local or general anesthetic is selected from the group consisting of diethyl ether, vinyl ether, halothane, chloroform, methoxyflurane, enflurane, trichloroethylene, isoflurane, desflurane, sevoflurane, methohexital, hexobarbital, thiopental, narcobarbital, fentanyl, alfentanil, sufentanil, phenoperidine, anileridine, remifentanil, droperidol, ketamine, propanidid, alfaxalone, etomidate, propofol, hydroxybutyric acid, nitrous oxide, esketamine, metabutethamine, procaine, tetracaine, chloroprocaine, benzocaine, bupivacaine, lidocaine, mepivacaine, prilocaine, butanilicaine, cinchocaine, etidocaine, articaine, ropivacaine, levobupivacaine, cocaine, ethyl chloride, dyclonine, phenol, and capsaicin.

24. The method as recited in claim 17, further resulting in at least one effect selected from the group consisting of:
a. decreased inter-individual variation in plasma levels of said compound or a metabolite thereof as compared to the non-isotopically enriched compound;
b. increased average plasma levels of said compound per dosage unit thereof as compared to the non-isotopically enriched compound;
c. decreased average plasma levels of at least one metabolite of said compound per dosage unit thereof as compared to the non-isotopically enriched compound;
d. increased average plasma levels of at least one metabolite of said compound per dosage unit thereof as compared to the non-isotopically enriched compound; and
e. an improved clinical effect during the treatment in said subject per dosage unit thereof as compared to the non-isotopically enriched compound.

25. The method as recited in claim 17, further resulting in at least two effects selected from the group consisting of:
a. decreased inter-individual variation in plasma levels of said compound or a metabolite thereof as compared to the non-isotopically enriched compound;
b. increased average plasma levels of said compound per dosage unit thereof as compared to the non-isotopically enriched compound;
c. decreased average plasma levels of at least one metabolite of said compound per dosage unit thereof as compared to the non-isotopically enriched compound;
d. increased average plasma levels of at least one metabolite of said compound per dosage unit thereof as compared to the non-isotopically enriched compound; and
e. an improved clinical effect during the treatment in said subject per dosage unit thereof as compared to the non-isotopically enriched compound.

26. The method as recited in claim 17, wherein the method affects a decreased metabolism of the compound per dosage unit thereof by at least one polymorphically-expressed cytochrome P₄₅₀ isoform in the subject, as compared to the corresponding non-isotopically enriched compound.

27. The method as recited in claim 26, wherein the cytochrome P₄₅₀ isoform is selected from the group consisting of CYP2C8, CYP2C9, CYP2C19, and CYP2D6.

28. The method as recited claim 17, wherein said compound is characterized by decreased inhibition of at least one cytochrome P₄₅₀ or monoamine oxidase isoform in said subject per dosage unit thereof as compared to the non-isotopically enriched compound.

29. The method as recited in claim 28, wherein said cytochrome P₄₅₀ or monoamine oxidase isoform is selected from the group consisting of CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, CYP51, MAO_{A}, and MAO_{B}.

30. The method as recited in claim 17, wherein the method reduces a deleterious change in a diagnostic hepatobiliary function endpoint, as compared to the corresponding non-isotopically enriched compound.

31. The method as recited in claim 30, wherein the diagnostic hepatobiliary function endpoint is selected from the group consisting of alanine aminotransferase (“ALT”), serum glutamic-pyruvic transaminase (“SGPT”), aspartate aminotransferase (“AST,” “SGOT”), ALT/AST ratios, serum aldolase, alkaline phosphatase (“ALP”), ammonia levels, bilirubin, gamma-glutamyl transpeptidase (“GGTP,” “γ-GTP,” “GGT”), leucine aminopeptidase (“LAP”), liver biopsy, liver ultrasonography, liver nuclear scan, 5′-nucleotidase, and blood protein.

32. A compound for use as a medicament, having structural Formula I: 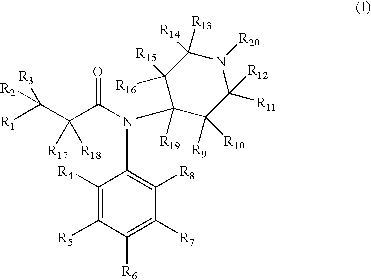
or a pharmaceutically acceptable salt thereof, wherein:
R₁-R₁₈, R₂₁-R₂₈, R₃₀-R₅₀ are independently selected from the group consisting of hydrogen and deuterium;
R₁₉ is selected from the group consisting of hydrogen, deuterium, and 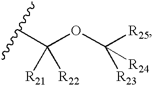
R₂₀ is selected from the group consisting of consisting of hydrogen, deuterium, 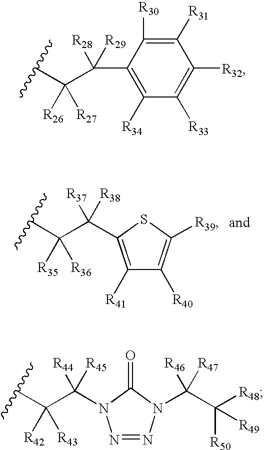
R₂₉ is selected from the group consisting of consisting of hydrogen, deuterium, hydroxyl, and —O-D; and
at least one of R₁-R₅₀ is deuterium.

33. A compound use in the manufacture of a medicament for the prevention or treatment of a disorder ameliorated by the modulation of opioid receptors, having structural Formula I: 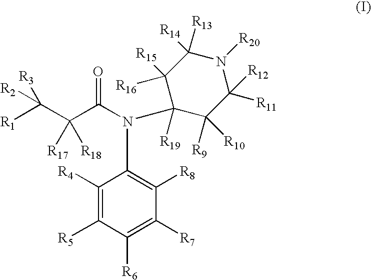
or a pharmaceutically acceptable salt thereof, wherein:
R₁-R₁₈, R₂₁-R₂₈, R₃₀-R₅₀ are independently selected from the group consisting of hydrogen and deuterium;
R₁₉ is selected from the group consisting of hydrogen, deuterium, and 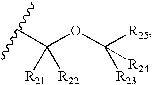
R₂₀ is selected from the group consisting of consisting of hydrogen, deuterium, 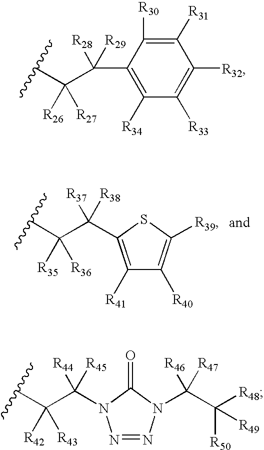
R₂₉ is selected from the group consisting of consisting of hydrogen, deuterium, hydroxyl, and —O-D; and
at least one of R₁-R₅₀ is deuterium.
